SEQUENCE LISTING (220 sequences)

SEQ ID NO: 1 - protein, 15 aa, Artificial Sequence
  Synthetic sequence
  VARIANT (1)..(1): Xaa = any amino acid
  VARIANT (2)..(2): Xaa = any uncharged amino aicd
  VARIANT (3)..(3): Xaa = any amino acid
  VARIANT (5)..(5): Xaa = any uncharged amino aicd
  VARIANT (6)..(6): Xaa = any uncharged, nonpolar amino acid
  VARIANT (7)..(7): Xaa = any amino acid
  VARIANT (9)..(9): Xaa = any uncharged, nonpolar amino acid
  VARIANT (10)..(10): Xaa = any amino acid
  VARIANT (11)..(11): Xaa = any uncharged, nonpolar amino acid
  VARIANT (12)..(12): Xaa = any uncharged amino aicd
  VARIANT (13)..(13): Xaa = any uncharged, nonpolar amino acid
  VARIANT (14)..(15): Xaa = any amino acid
XXXDXXXNXXXXXXX

SEQ ID NO: 2 - protein, 15 aa, Artificial Sequence
  Synthetic sequence (EP003 IL2Rbeta BR2)
GVTDSISNAIVLARE

SEQ ID NO: 3 - protein, 15 aa, Artificial Sequence
  Synthetic sequence (EP008 IL2Rbeta BR2)
KWGDAVSNARVLAGE

SEQ ID NO: 4 - protein, 15 aa, Artificial Sequence
  Synthetic sequence (EP007 IL2Rbeta BR2)
KWGDAVSNARVLAGA

SEQ ID NO: 5 - protein, 15 aa, Artificial Sequence
  Synthetic sequence (EP0021 IL2Rbeta BL2)
TLMDTTDNIGVLVRE

SEQ ID NO: 6 - protein, 15 aa, Artificial Sequence
  Synthetic sequence (EP002 IL2Rbeta BR2)
EPSDVISNINVLVQE

SEQ ID NO: 7 - protein, 15 aa, Artificial Sequence
  Synthetic sequence (EP018 IL2Rbeta BR2)
SPQDSIENISVLVRE

SEQ ID NO: 8 - protein, 15 aa, Artificial Sequence
  Synthetic sequence (EP011 IL2Rbeta BR2)
WASDSIENITLLIQE

SEQ ID NO: 9 - protein, 15 aa, Artificial Sequence
  Synthetic sequence (EP004 IL2Rbeta BR2)
CPTDTIENITVLIQE

SEQ ID NO: 10 - protein, 15 aa, Artificial Sequence
  Synthetic sequence (EP016 IL2Rbeta BR2)
RYKDSLENMQIIIQE

SEQ ID NO: 11 - protein, 15 aa, Artificial Sequence
  Synthetic sequence (EP001 IL2Rbeta BR2)
TARDAVDNMRVIIQE

SEQ ID NO: 12 - protein, 15 aa, Artificial Sequence
  Synthetic sequence (EP017 IL2Rbeta BR2)
TPRDVVENMNVLVLE

SEQ ID NO: 13 - protein, 15 aa, Artificial Sequence
  Synthetic sequence (EP012 IL2Rbeta BR2)
TPSDVIENMEVLILD

SEQ ID NO: 14 - protein, 15 aa, Artificial Sequence
  Synthetic sequence (EP013 IL2Rbeta BR2)
TPSDAIENINVLIRE

SEQ ID NO: 15 - protein, 15 aa, Artificial Sequence
  Synthetic sequence (EP015 IL2Rbeta BR2)
TPSDVIENITVLVQE

SEQ ID NO: 16 - protein, 15 aa, Artificial Sequence
  Synthetic sequence (EP006 IL2Rbeta BR2)
GVGDTIDNINVLVKE

SEQ ID NO: 17 - protein, 15 aa, Artificial Sequence
  Synthetic sequence (EP019 IL2Rbeta BR2)
IGRDSIDNIKVIVQE

SEQ ID NO: 18 - protein, 15 aa, Artificial Sequence
  Synthetic sequence (EP009 IL2Rbeta BR2)
WATDTIRNVEVLVQE

SEQ ID NO: 19 - protein, 15 aa, Artificial Sequence
  Synthetic sequence (EP005 IL2Rbeta BR2)
TAEDVVTNITVLVQE

SEQ ID NO: 20 - protein, 15 aa, Artificial Sequence
  Synthetic sequence (EP010 IL2Rbeta BR2)
TAEDVISNIRVNVQE

SEQ ID NO: 21 - protein, 15 aa, Artificial Sequence
  Synthetic sequence (EP014 IL2Rbeta BR2)
TPSDVIDNVSITVQE

SEQ ID NO: 22 - protein, 133 aa, Artificial Sequence
  Synthetic sequence (IL2Rbeta agonist)
  VARIANT (81)..(81): Xaa = any amino acid
  VARIANT (82)..(82): Xaa = any uncharged amino acid
  VARIANT (83)..(83): Xaa = any amino acid
  VARIANT (85)..(85): Xaa = any uncharged amino acid
  VARIANT (86)..(86): Xaa = any uncharged, nonpolar amino acid
  VARIANT (87)..(87): Xaa = any amino acid
  VARIANT (89)..(89): Xaa = any uncharged, nonpolar amino acid
  VARIANT (90)..(90): Xaa = any amino acid
  VARIANT (91)..(91): Xaa = any uncharged, nonpolar amino acid
  VARIANT (92)..(92): Xaa = any uncharged amino acid
  VARIANT (93)..(93): Xaa = any uncharged, nonpolar amino acid
  VARIANT (94)..(95): Xaa = any amino acid
APTSSSTKKTQLQLEHLLLDLQMILNGINNYKNPKLTRMLTFKFYMPKKATELKHLQCLEEELKPLEEVLNLAQSKNFHLXXXDXXXNXXXXXXXLKGSETTFMCEYADETATIVEFLNRWITFCQSIISTLT

SEQ ID NO: 23 - protein, 133 aa, Artificial Sequence
  Synthetic sequence EP003
APTSSSTKKTQLQLEHLLLDLQMILNGINNYKNPKLTRMLTFKFYMPKKATELKHLQCLEEELKPLEEVLNLAQSKNFHLGVTDSISNAIVLARELKGSETTFMCEYADETATIVEFLNRWITFCQSIISTLT

SEQ ID NO: 24 - protein, 133 aa, Artificial Sequence
  Synthetic sequence EP008
APTSSSTKKTQLQLEHLLLDLQMILNGINNYENPKLTRMLTFKFYMPKKATELKHLQCLEEELKPLEEVLNLAQSKNFHLKWGDAVSNARVLAGELKGSETTFMCEYADETATIVEFLNRWITFCQSIISTLT

SEQ ID NO: 25 - protein, 133 aa, Artificial Sequence
  Synthetic sequence EP007
APTSSSTKETQLQLKHLLLDLQMILNGINNYKNPKLTRMLTFKFYMPKKATELKHLQCLEEELKPLEEVLNLAQSKNFHLKWGDAVSNARVLAGALKGSETTFMCEYADETATIVEFLNRWITFCQSIISTLT

SEQ ID NO: 26 - protein, 133 aa, Artificial Sequence
  Synthetic sequence EP020
APTSSSTKKTQLQLEHLLLDLQMILNGINNYKNPKLTRMLTFKFYMPKKATELKHLQCLEEELKPLEEVLNLAQSKNFHLTLMDTTDNIGVLVRELKGSETTFMCEYADETATIVEFLNRWITFCQSIISTLT

SEQ ID NO: 27 - protein, 133 aa, Artificial Sequence
  Synthetic sequence EP002
APTSSSTKKTQLQLEHLLLDLQMILNGINNYKNPKLTEMLTFKFYMPKKATELKHLQCLEEELKPLEEVLNLAQSKNFHLEPSDVISNINVLVQELKGSETTFMCEYADETATIVEFLNRWITFCQSIISTLT

SEQ ID NO: 28 - protein, 133 aa, Artificial Sequence
  Synthetic sequence EP018
APTSSSTKETQLQLKHLLLDLQMILNGINNYKNPKLTRMLTFKFYMPKKATELKHLQCLEEELKPLEEVLNLAQSKNFHLSPQDSIENISVLVRELKGSETTFMCEYADETATIVEFLNRWITFCQSIISTLT

SEQ ID NO: 29 - protein, 133 aa, Artificial Sequence
  Synthetic sequence EP011
APTSSSTKKTQLQLEHLLLDLQMILNGINNYKNPKLTRMLTFKFYMPKKATELKHLQCLEEELKPLEEVLNLAQSKNFHLWASDSIENITLLIQELKGSETTFMCEYADETATIVEFLNRWITFCQSIISTLT

SEQ ID NO: 30 - protein, 133 aa, Artificial Sequence
  Synthetic sequence EP004
APTSSSTKKTQLQLEHLLLDLQMILNGINNYKNPKLTRMLTFKFYMPKKATELKHLQCLEEELKPLEEVLNLAQSKNFHLCPTDTIENITVLIQELKGSETTFMCEYADETATIVEFLNRWITFCQSIISTLT

SEQ ID NO: 31 - protein, 133 aa, Artificial Sequence
  Synthetic sequence EP016
APTSSSTKKTQLQLEHLLLDLQMILNGINNYKNPKLTRMLTFKFYMPKKATELKHLQCLEEELKPLEEVLNLAQSKNFHLRYKDSLENMQIIIQELKGSETTFMCEYADETATIVEFLNRWITFCQSIISTLT

SEQ ID NO: 32 - protein, 133 aa, Artificial Sequence
  Synthetic sequence EP001
APTSSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFCQSIISTLT

SEQ ID NO: 33 - protein, 133 aa, Artificial Sequence
  Synthetic sequence EP017
APTSSSTKKTQLQLEHLLLDLQMILNGINNYKNPKLTRMLTFKFYMPKKATELKHLQCLEEELKPLEEVLNLAQSKNFHLTPRDVVENMNVLVLELKGSETTFMCEYADETATIVEFLNRWITFCQSIISTLT

SEQ ID NO: 34 - protein, 133 aa, Artificial Sequence
  Synthetic sequence EP012
APTSSSTKKTQLQLEHLLLDLQMILNGINNYKNPKLTRMLTFKFYMPKKATELKHLQCLEEELKPLEEVLNLAQSKNFHLTPSDVIENMEVLILDLKGSETTFMCEYADETATIVEFLNRWITFCQSIISTLT

SEQ ID NO: 35 - protein, 133 aa, Artificial Sequence
  Synthetic sequence EP013
APTSSSTKKTQLQLEHLLLDLQMILNGINNYKNPKLTRMLTFKFYMPKKATELKHLQCLEEELKPLEEVLNLAQSKNFHLTPSDAIENINVLIRELKGSETTFMCEYADETATIVEFLNRWITFCQSIISTLT

SEQ ID NO: 36 - protein, 133 aa, Artificial Sequence
  Synthetic sequence EP015
APTSSSTKKTQLQLEHLLLDLQMILNGINNYKNPKLTRMLTFKFYMPKKATELKHLQCLEEELKPLEEVLNLAQSKNFHLTPSDVIENITVLVQELKGSETTFMCEYADETATIVEFLNRWITFCQSIISTLT

SEQ ID NO: 37 - protein, 133 aa, Artificial Sequence
  Synthetic sequence EP006
APTSSSTKKTQLQLEHLLLDLQMILNGINNYKNPKLTRMLTFKFYMPKKATELKHLQCLEEELKPLEEVLNLAQSKNFHLGVGDTIDNINVLVKELKGSETTFMCEYADETATIVEFLNRWITFCQSIISTLT

SEQ ID NO: 38 - protein, 133 aa, Artificial Sequence
  Synthetic sequence EP019
APTSSSTKKTQLQLEHLLLDLQMILNGINNYKNPKLTRMLTFKFYMPKKATELKHLQCLEEELKPLEEVLNLAQSKNFHLIGRDSIDNIKVIVQELKGSETTFMCEYADETATIVEFLNRWITFCQSIISTLT

SEQ ID NO: 39 - protein, 133 aa, Artificial Sequence
  Synthetic sequence EP009
APTSSSTKKTQLQLEHLLLDLQMILNGINNYKNPKLTRMLTFKFYMPKKATELKHLQCLEEELKPLEEVLNLAQSKNFHLWATDTIRNVEVLVQELKGSETTFMCEYADETATIVEFLNRWITFCQSIISTLT

SEQ ID NO: 40 - protein, 133 aa, Artificial Sequence
  Synthetic sequence EP005
APTSSSTKKTQLQLEHLLLDLQMILNGINNYKNPKLTRMLTFKFYMPKKATELKHLQCLEEELKPLEEVLNLAQSKNFHLTAEDVVTNITVLVQELKGSETTFMCEYADETATIVEFLNRWITFCQSIISTLT

SEQ ID NO: 41 - skipped

SEQ ID NO: 42 - protein, 133 aa, Artificial Sequence
  Synthetic sequence EP014
APTSSSTKKTQLQLEHLLLDLQMILNGINNYKNPKLTRMLTFKFYMPKKATELKHLQCLEEELKPLEEVLNLAQSKNFHLTPSDVIDNVSITVQELKGSETTFMCEYADETATIVEFLNRWITFCQSIISTLT

SEQ ID NO: 43 - protein, 365 aa, Artificial Sequence
  Synthetic sequence IL2Rbeta agonist-Fc fusion
  VARIANT (81)..(81): Xaa = any amino acid
  VARIANT (82)..(82): Xaa = any uncharged amino acid
  VARIANT (83)..(83): Xaa = any amino acid
  VARIANT (85)..(85): Xaa = any uncharged amino acid
  VARIANT (86)..(86): Xaa = any uncharged, nonpolar residue
  VARIANT (87)..(87): Xaa = any amino acid
  VARIANT (89)..(89): Xaa = any uncharged, nonpolar residue
  VARIANT (90)..(90): Xaa = any amino acid
  VARIANT (91)..(91): Xaa = any uncharged, nonpolar residue
  VARIANT (92)..(92): Xaa = any uncharged amino acid
  VARIANT (93)..(93): Xaa = any uncharged, nonpolar residue
  VARIANT (94)..(95): Xaa = any amino acid
APTSSSTKKTQLQLEHLLLDLQMILNGINNYKNPKLTRMLTFKFYMPKKATELKHLQCLEEELKPLEEVLNLAQSKNFHLXXXDXXXNXXXXXXXLKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 44 - protein, 365 aa, Artificial Sequence
  Synthetic sequence EP003-Fc fusion, EP082
APTSSSTKKTQLQLEHLLLDLQMILNGINNYKNPKLTRMLTFKFYMPKKATELKHLQCLEEELKPLEEVLNLAQSKNFHLGVTDSISNAIVLARELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 45 - protein, 367 aa, Artificial Sequence
  Synthetic sequence EP008-Fc fusion, EP086
APTSSSTKKTQLQLEHLLLDLQMILNGINNYENPKLTRMLTFKFYMPKKATELKHLQCLEEELKPLEEVLNLAQSKNFHLKWGDAVSNARVLAGELKGSETTFMCEYADETATIVEFLNRWITFCQSIISTLTRTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 46 - protein, 365 aa, Artificial Sequence
  Synthetic sequence EP007-Fc fusion, EP080
APTSSSTKETQLQLKHLLLDLQMILNGINNYKNPKLTRMLTFKFYMPKKATELKHLQCLEEELKPLEEVLNLAQSKNFHLKWGDAVSNARVLAGALKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 47 - protein, 365 aa, Artificial Sequence
  Synthetic sequence EP020-Fc fusion, EP098
APTSSSTKKTQLQLEHLLLDLQMILNGINNYKNPKLTRMLTFKFYMPKKATELKHLQCLEEELKPLEEVLNLAQSKNFHLTLMDTTDNIGVLVRELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 48 - protein, 365 aa, Artificial Sequence
  Synthetic sequence EP002-Fc fusion, EP083
APTSSSTKKTQLQLEHLLLDLQMILNGINNYKNPKLTEMLTFKFYMPKKATELKHLQCLEEELKPLEEVLNLAQSKNFHLEPSDVISNINVLVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 49 - protein, 365 aa, Artificial Sequence
  Synthetic sequence EP018-Fc fusion, EP096
APTSSSTKETQLQLKHLLLDLQMILNGINNYKNPKLTRMLTFKFYMPKKATELKHLQCLEEELKPLEEVLNLAQSKNFHLSPQDSIENISVLVRELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 50 - protein, 366 aa, Artificial Sequence
  Synthetic sequence EP011-Fc fusion, EP089
ATPTSSSTKKTQLQLEHLLLDLQMILNGINNYKNPKLTRMLTFKFYMPKKATELKHLQCLEEELKPLEEVLNLAQSKNFHLWASDSIENITLLIQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 51 - protein, 365 aa, Artificial Sequence
  Synthetic sequence EP004-Fc fusion, EP078
APTSSSTKKTQLQLEHLLLDLQMILNGINNYKNPKLTRMLTFKFYMPKKATELKHLQCLEEELKPLEEVLNLAQSKNFHLTAEDVVTNITVLVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 52 - protein, 365 aa, Artificial Sequence
  Synthetic sequence EP016-Fc fusion, EP094
APTSSSTKKTQLQLEHLLLDLQMILNGINNYKNPKLTRMLTFKFYMPKKATELKHLQCLEEELKPLEEVLNLAQSKNFHLRYKDSLENMQIIIQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 53 - protein, 365 aa, Artificial Sequence
  Synthetic sequence EP001-Fc fusion, EP079
APTSSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 54 - protein, 367 aa, Artificial Sequence
  Synthetic sequence EP017-Fc fusion, EP095
APTSSSTKKTQLQLEHLLLDLQMILNGINNYENPKLTRMLTFKFYMPKKATELKHLQCLEEELKPLEEVLNLAQSKNFHLTPRDVVENMNVLVLELKGSETTFMCEYADETATIVEFLNRWITFCQSIISTLTRTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 55 - protein, 365 aa, Artificial Sequence
  Synthetic sequence EP012-Fc fusion, EP090
APTSSSTKETQLQLKHLLLDLQMILNGINNYKNPKLTRMLTFKFYMPKKATELKHLQCLEEELKPLEEVLNLAQSKNFHLTPSDVIENMEVLILDLKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 56 - protein, 365 aa, Artificial Sequence
  Synthetic sequence EP013-Fc fusion, EP091
APTSSSTKKTQLQLEHLLLDLQMILNGINNYKNPKLTEMLTFKFYMPKKATELKHLQCLEEELKPLEEVLNLAQSKNFHLTPSDAIENINVLIRELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 57 - protein, 365 aa, Artificial Sequence
  Synthetic sequence EP015-Fc fusion, EP093
APTSSSTKETQLQLKHLLLDLQMILNGINNYKNPKLTRMLTFKFYMPKKATELKHLQCLEEELKPLEEVLNLAQSKNFHLTPSDVIENITVLVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 58 - protein, 366 aa, Artificial Sequence
  Synthetic sequence EP006-Fc fusion, EP081
ATPTSSSTKKTQLQLEHLLLDLQMILNGINNYKNPKLTRMLTFKFYMPKKATELKHLQCLEEELKPLEEVLNLAQSKNFHLGVGDTIDNINVLVKELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 59 - protein, 365 aa, Artificial Sequence
  Synthetic sequence EP019-Fc fusion, EP097
APTSSSTKKTQLQLEHLLLDLQMILNGINNYKNPKLTRMLTFKFYMPKKATELKHLQCLEEELKPLEEVLNLAQSKNFHLIGRDSIDNIKVIVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 60 - protein, 365 aa, Artificial Sequence
  Synthetic sequence EP009-Fc fusion, EP087
APTSSSTKKTQLQLEHLLLDLQMILNGINNYKNPKLTRMLTFKFYMPKKATELKHLQCLEEELKPLEEVLNLAQSKNFHLWATDTIRNVEVLVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 61 - protein, 365 aa, Artificial Sequence; identical to SEQ ID NO: 51
  Synthetic sequence EP005-Fc fusion, EP084

SEQ ID NO: 62 - protein, 365 aa, Artificial Sequence
  Synthetic sequence EP010-Fc fusion, EP088
APTSSSTKKTQLQLEHLLLDLQMILNGINNYKNPKLTRMLTFKFYMPKKATELKHLQCLEEELKPLEEVLNLAQSKNFHLTAEDVISNIRVNVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 63 - protein, 365 aa, Artificial Sequence
  Synthetic sequence EP014-Fc fusion, EP092
APTSSSTKKTQLQLEHLLLDLQMILNGINNYKNPKLTRMLTFKFYMPKKATELKHLQCLEEELKPLEEVLNLAQSKNFHLTPSDVIDNVSITVQELKGSETTFMCEYADETATIVEFLNRWITFCQSIISTLTEPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 64 - protein, 232 aa, Artificial Sequence
  Synthetic sequence Fc domain
EPKSCDKTHTCPLCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 65 - protein, 133 aa, Homo sapiens [sequence omitted]

SEQ ID NO: 66 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 67 - protein, 12 aa, Artificial Sequence
  Synthetic sequence WT IL2 IL2R beta binding loop 1
QLQLEHLLLDLQ

SEQ ID NO: 68 - protein, 15 aa, Artificial Sequence
  Synthetic sequence WT IL2 IL2R beta binding loop 2
RPRDLISNINVIVLE

SEQ ID NO: 69 - DNA, 75 bp, Artificial Sequence
  Synthetic sequence Mutagenic oligo 1
agttctacaaagaaaacacagctacaactggagcatttactgctggatttacagatgattttgaatggaattaat

SEQ ID NO: 70 - DNA, 81 bp, Artificial Sequence
  Synthetic sequence Mutagenic oligo 2
agcaaaaactttcacttaagacccagggacttaatcagcaatatcaacgtaatagttctggaactaaagggatctgaaaca

SEQ ID NO: 71 - DNA, 60 bp, Artificial Sequence
  Synthetic sequence Mutagenic oligo 3
caattactatttacaattacaatggctagcgcacctacttcaagttctacaaagaaaaca

SEQ ID NO: 72 - DNA, 75 bp, Artificial Sequence
  Synthetic sequence Mutagenic oligo 4
cttgggcatgtaaaacttaaatgtgagcatcctggtgagtttgggattcttgtaattattaattccattcaaaat

SEQ ID NO: 73 - DNA, 70 bp, Artificial Sequence
  Synthetic sequence Mutagenic oligo 5
aagttttacatgcccaagaaggccacagaactgaaacatcttcagtgtctagaagaagaactcaaacctc

SEQ ID NO: 74 - DNA, 60 bp, Artificial Sequence
  Synthetic sequence Mutagenic oligo 6
taagtgaaagtttttgctttgagctaaatttagcacttcctccagaggtttgagttcttc

SEQ ID NO: 75 - DNA, 75 bp, Artificial Sequence
  Synthetic sequence Mutagenic oligo 7
ctaaagggatctgaaacaacattcatgtgtgaatatgctgatgagacagcaaccattgtagaatttctgaacaga

SEQ ID NO: 76 - DNA, 78 bp, Artificial Sequence
  Synthetic sequence Mutagenic oligo 8
agatggtgcagccacagttcgagtcagtgttgagatgatgctttgacaaaaggtaatccatctgttcagaaattctac

SEQ ID NO: 77 - DNA, 75 bp, Artificial Sequence; identical to SEQ ID NO: 69
  Synthetic sequence Mutagenic oligo 9

SEQ ID NO: 78 - DNA, 81 bp, Artificial Sequence; identical to SEQ ID NO: 70
  Synthetic sequence Mutagenic oligo 10

SEQ ID NO: 79 - DNA, 18 bp, Artificial Sequence
  Synthetic sequence Mutagenic oligo 11
aagttttacatgcccaag

SEQ ID NO: 80 - DNA, 17 bp, Artificial Sequence
  Synthetic sequence Mutagenic oligo 12
taagtgaaagtttttgc

SEQ ID NO: 81 - protein, 133 aa, Artificial Sequence
  Synthetic sequence EP100
APTSSSTKKTQLQLEHLLLDLQMILNGINNYKNPVLTRMLTIKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 82 - protein, 133 aa, Artificial Sequence
  Synthetic sequence EP101
APTSSSTKKTQLQLEHLLLDLQMILNGINNYKNPKLTRMLTLKFPMPKKATELKHLQCLEEELKPLEEVLNLAQSKNFHLRPRDLISNINVIVLELKGSETTFMCEYADETATIVEFLNRWITFSQSIISTLT

SEQ ID NO: 83 - protein, 133 aa, Artificial Sequence
  Synthetic sequence EP102
APTSSSTKKTQLQLEHLLLDLQMILNGINNYKNPDLTSMLAFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 84 - protein, 133 aa, Artificial Sequence
  Synthetic sequence EP103
SPTSSSTKKTQLQLEHLLLDLQMILNGINNYKNPGLTEMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 85 - protein, 133 aa, Artificial Sequence
  Synthetic sequence EP104
APTSSSTKKTQLQLEHLLLDLQMILNGINNYKNPSLTRMLTGKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 86 - protein, 133 aa, Artificial Sequence
  Synthetic sequence EP105
APTSSSTKKTQLQLEHLLLDLQMILNGINNYKNPELTIMLTPKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 87 - protein, 133 aa, Artificial Sequence
  Synthetic sequence EP106
APTSSSTKKTQLQLEHLLLDLQMILNGINNYKNPCLTAMLTLKFAMPKKATELKHLQCLEEELKPLEEVLNLAQSKNFHLRPRDLISNINVIVLELKGSETTFMCEYADETATIVEFLNRWITFSQSIISTLT

SEQ ID NO: 88 - protein, 133 aa, Artificial Sequence; identical to SEQ ID NO: 87
  Synthetic sequence EP107

SEQ ID NO: 89 - protein, 133 aa, Artificial Sequence
  Synthetic sequence EP108
APTSSSTKKTQLQLEHLLLDLQMILNGINNYKNPKLTRMLTHKFVMPKKATELKHLQCLEEELKPLEEVLNLAQSKNFHLRPRDLISNINVIVLELKGSETTFMCEYADETATIVEFLNRWITFSQSIISTLT

SEQ ID NO: 90 - protein, 133 aa, Artificial Sequence
  Synthetic sequence EP109
APTSSSTKKTQLQLEHLLLDLQMILNGINNYKNPCLTDMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 91 - protein, 133 aa, Artificial Sequence
  Synthetic sequence EP110
APTSSSTKKTQLQLEHLLLDLQMILNGINNYKNPLLTDMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 92 - protein, 133 aa, Artificial Sequence
  Synthetic sequence EP111
APTSSSTKKTQLQLEHLLLDLQMILNGINNYKNPLLTDMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 93 - protein, 133 aa, Artificial Sequence
  Synthetic sequence EP112
APTSSSTKKTQLQLEHLLLDLQMILNGINNYKNPKLTDMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 94 - protein, 133 aa, Artificial Sequence
  Synthetic sequence EP113
APTSSSTKKTQLQLEHLLLDLQMILNGINNYKNPKLTYMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 95 - protein, 133 aa, Artificial Sequence
  Synthetic sequence EP114
APTSSSTKKTQLQLEHLLLDLQMILNGINNYKNPKLTRMLTFKFCMPKKATELKHLQCLEEELKPLEEVLNLAQSKNFHLRPRDLISNINVIVLELKGSETTFMCEYADETATIVEFLNRWITFSQSIISTLT

SEQ ID NO: 96 - protein, 133 aa, Artificial Sequence
  Synthetic sequence EP115
APTSSSTKKTQLQLEHLLLDLQMILNGLNNYKNPKLTSMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 97 - protein, 133 aa, Artificial Sequence; identical to SEQ ID NO: 96
  Synthetic sequence EP116

SEQ ID NO: 98 - protein, 133 aa, Artificial Sequence
  Synthetic sequence EP117
APTSSSTKKTQLQLEHLLLDLQMILNGINNYKNPKLTYMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 99 - protein, 133 aa, Artificial Sequence; identical to SEQ ID NO: 98
  Synthetic sequence EP118

SEQ ID NO: 100 - protein, 133 aa, Artificial Sequence
  Synthetic sequence EP119
APTSSSTKKTQLQLEHLLLDLQMILNGINNYKNPKLTGMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 101 - protein, 133 aa, Artificial Sequence
  Synthetic sequence EP120
APTSSSTKKTQLQLEHLLLDLQMILNGINNYKNPKLTVMLTFKFTMPKKATELKHLQCLEEELKPLEEVLNLAQSKNFHLRPRDLISNINVIVLELKGSETTFMCEYADETATIVEFLNRWITFSQSIISTLT

SEQ ID NO: 102 - protein, 133 aa, Artificial Sequence
  Synthetic sequence EP121
APTSSSTKKTQLQLEHLLLDLQMILNGINNYKNPKLTVMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 103 - protein, 133 aa, Artificial Sequence
  Synthetic sequence EP122
APTSSSTKKTQLQLEHLLLDLQMILNGINNYKNPKLTVMLTFKFPMPKKATELKHLQCLEEELKPLEEVLNLAQSKNFHLRPRDLISNINVIVLELKGSETTFMCEYADETATIVEFLNRWITFSQSIISTLT

SEQ ID NO: 104 - protein, 133 aa, Artificial Sequence
  Synthetic sequence EP123
APTSSSTKKTQLQLEHLLLDLQMILNGINNYKNPKLTVMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 105 - protein, 133 aa, Artificial Sequence
  Synthetic sequence EP124
APTSSSTKKTQLQLEHLLLDLQMILNGINNYKNPKLTCMLTFKFAMPKKATELKHLQCLEEELKPLEEVLNLAQSKNFHLRPRDLISNINVIVLELKGSETTFMCEYADETATIVEFLNRWITFSQSIISTLT

SEQ ID NO: 106 - protein, 133 aa, Artificial Sequence
  Synthetic sequence EP125
APTSSSTKKTQLQLEHLLLDLQMILNGINNYKNPKLTNMLTFKFAMPKKATELKHLQCLEEELKPLEEVLNLAQSKNFHLRPRDLISNINVIVLELKGSETTFMCEYADETATIVEFLNRWITFSQSIISTLT

SEQ ID NO: 107 - protein, 133 aa, Artificial Sequence
  Synthetic sequence EP126
APTSSSTKKTQLQLEHLLLDLQMILNGINNYKNPKLTNMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 108 - protein, 362 aa, Artificial Sequence
  Synthetic sequence EP213 (WT IL2-IgG4-Fc, Knob mutations: S354C, T366W, K409A)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 109 - protein, 229 aa, Artificial Sequence
  Synthetic sequence EP214 (IgG4-Fc, Hole mutations: Y349C, T366S, L368A, F405K, Y407V)
ESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFKLVSRLTVDKSRWQEGNVFSCSVMHEALHNHYTQKSLSLSLGK

SEQ ID NO: 110 - protein, 362 aa, Artificial Sequence
  Synthetic sequence EP215 (EP001-IgG4-Fc, Knob mutations: S354C, T366W, K409A)
APTSSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 111 - protein, 361 aa, Artificial Sequence
  Synthetic sequence EP216 (EP003-IgG4-Fc, Knob mutations: S354C, T366W, K409A
APTSSSTKKTQLQLEHLLLDLQMILNGINNYKNPKLTRMLTFKFYMPKKATELKHLQCLEEELKPLEEVLNLAQSKNFHLGVTDSISNAIVLARELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLG

SEQ ID NO: 112 - protein, 362 aa, Artificial Sequence
  Synthetic sequence EP219
APTSSSTKKTQLQLEHLLLDLQMILNGINNYKNPKLTRMLTAKFYMPKKATELKHLQCLEEELKPLEEVLNLAQSKNFHLGVTDSISNAIVLARELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 113 - protein, 362 aa, Artificial Sequence
  Synthetic sequence EP220
APTSSSTKKTQLQLEHLLLDLQMILNGINNYKNPKLTRMLTAKFYMPKKATELKHLQCLEEELKPLEEVLNLAQSKNFHLTARDAVDNMRVIIQELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 114 - protein, 362 aa, Artificial Sequence
  Synthetic sequence EP221
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 115 - protein, 362 aa, Artificial Sequence
  Synthetic sequence EP221
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFSQSIISTLTESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSALTVDKSRWQEGNVFSCSVMHEALHNHYTQKSLSLSLGK

SEQ ID NO: 116 - protein, 141 aa, Artificial Sequence
  Synthetic sequence EP223
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFSQSIISTLTGSHHHHHH

SEQ ID NO: 117 - protein, 141 aa, Artificial Sequence
  Synthetic sequence EP224
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFSQSIISTLTGSHHHHHH

SEQ ID NO: 118 - protein, 161 aa, Artificial Sequence
  Synthetic sequence EP225
METDTLLLWVLLLWVPGSTGAPTSSSTKKTQLQLEHLLLDLQMILNGINNYKNPKLTRMLTAKFYMPKKATELKHLQCLEEELKPLEEVLNLAQSKNFHLTARDAVDNMRVIIQELKGSETTFMCEYADETATIVEFLNRWITFSQSIISTLTGSHHHHHH

SEQ ID NO: 119 - protein, 141 aa, Artificial Sequence
  Synthetic sequence EP226
APTSSSTKKTQLQLEHLLLDLQMILNGINNYKNPKLTRMLTAKFYMPKKATELKHLQCLEEELKPLEEVLNLAQSKNFHLGVTDSISNAIVLARELKGSETTFMCEYADETATIVEFLNRWITFSQSIISTLTGSHHHHHH

SEQ ID NO: 120 - protein, 156 aa, Artificial Sequence
  Synthetic sequence EP239
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTRTVAAPSDYKDDDDKGGHHHHHH

SEQ ID NO: 121 - protein, 161 aa, Artificial Sequence
  Synthetic sequence EP242
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAISNIRVIVQELKGSETTFMCEYADETATIVEFLNRWITFSQSIISTLTGSHHHHHH

SEQ ID NO: 122 - protein, 161 aa, Artificial Sequence
  Synthetic sequence EP243
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRPRDAIDNIRVIIQELKGSETTFMCEYADETATIVEFLNRWITFSQSIISTLTGSHHHHHH

SEQ ID NO: 123 - protein, 161 aa, Artificial Sequence
  Synthetic sequence EP244
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRARDLIDNIRVIIQELKGSETTFMCEYADETATIVEFLNRWITFSQSIISTLTGSHHHHHH

SEQ ID NO: 124 - protein, 161 aa, Artificial Sequence
  Synthetic sequence EP245
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRARDAISNIRVIIQELKGSETTFMCEYADETATIVEFLNRWITFSQSIISTLTGSHHHHHH

SEQ ID NO: 125 - protein, 161 aa, Artificial Sequence
  Synthetic sequence EP246
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRARDAIDNINVIIQELKGSETTFMCEYADETATIVEFLNRWITFSQSIISTLTGSHHHHHH

SEQ ID NO: 126 - protein, 161 aa, Artificial Sequence
  Synthetic sequence EP247
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRARDAIDNIRVIVQELKGSETTFMCEYADETATIVEFLNRWITFSQSIISTLTGSHHHHHH

SEQ ID NO: 127 - protein, 161 aa, Artificial Sequence
  Synthetic sequence EP248
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLRARDAIDNIRVIILELKGSETTFMCEYADETATIVEFLNRWITFSQSIISTLTGSHHHHHH

SEQ ID NO: 128 - protein, 161 aa, Artificial Sequence
  Synthetic sequence EP249
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTPRDLIDNIRVIIQELKGSETTFMCEYADETATIVEFLNRWITFSQSIISTLTGSHHHHHH

SEQ ID NO: 129 - protein, 161 aa, Artificial Sequence
  Synthetic sequence EP250
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTPRDAISNIRVIIQELKGSETTFMCEYADETATIVEFLNRWITFSQSIISTLTGSHHHHHH

SEQ ID NO: 130 - protein, 161 aa, Artificial Sequence
  Synthetic sequence EP251
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTPRDAIDNINVIIQELKGSETTFMCEYADETATIVEFLNRWITFSQSIISTLTGSHHHHHH

SEQ ID NO: 131 - protein, 161 aa, Artificial Sequence
  Synthetic sequence EP252
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 132 - protein, 161 aa, Artificial Sequence
  Synthetic sequence EP253
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 133 - protein, 161 aa, Artificial Sequence
  Synthetic sequence EP254
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDLISNIRVIIQELKGSETTFMCEYADETATIVEFLNRWITFSQSIISTLTGSHHHHHH

SEQ ID NO: 134 - protein, 161 aa, Artificial Sequence
  Synthetic sequence EP255
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDLIDNINVIIQELKGSETTFMCEYADETATIVEFLNRWITFSQSIISTLTGSHHHHHH

SEQ ID NO: 135 - protein, 161 aa, Artificial Sequence
  Synthetic sequence EP256
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDLIDNIRVIVQELKGSETTFMCEYADETATIVEFLNRWITFSQSIISTLTGSHHHHHH

SEQ ID NO: 136 - protein, 161 aa, Artificial Sequence
  Synthetic sequence EP257
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDLIDNIRVIILELKGSETTFMCEYADETATIVEFLNRWITFSQSIISTLTGSHHHHHH

SEQ ID NO: 137 - protein, 161 aa, Artificial Sequence
  Synthetic sequence EP258
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 138 - protein, 161 aa, Artificial Sequence
  Synthetic sequence EP260
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 139 - protein, 161 aa, Artificial Sequence
  Synthetic sequence EP261
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAISNIRVIILELKGSETTFMCEYADETATIVEFLNRWITFSQSIISTLTGSHHHHHH

SEQ ID NO: 140 - protein, 161 aa, Artificial Sequence
  Synthetic sequence EP262
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAIDNINVIILELKGSETTFMCEYADETATIVEFLNRWITFSQSIISTLTGSHHHHHH

SEQ ID NO: 141 - protein, 161 aa, Artificial Sequence
  Synthetic sequence EP263
METDTLLLWVLLLWVPGSTGAPTSSSTKKTQLQLEHLLLDLQMILNGINNYKNPKLTRMLTFKFYMPKKATELKHLQCLEEELKPLEEVLNLAQSKNFHLTARDAIDNIRVIVLELKGSETTFMCEYADETATIVEFLNRWITFSQSIISTLTGSHHHHHH

SEQ ID NO: 142 - protein, 227 aa, Artificial Sequence
  Synthetic sequence EP280 (IgG1_Fc; Hole mutation: Y349C, T366S, L368A, F405K, Y407V; ADCC mutation: L234A, L235A, P329G)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 143 - protein, 360 aa, Artificial Sequence
  Synthetic sequence EP290 (WT-IL2-IgG1-Fc, T3A, C125S; Fc Knob mutation: S354C, T366W, K409A; ADCC mutation: L234A, L235A, P329G)
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 144 - protein, 360 aa, Artificial Sequence
  Synthetic sequence EP291 (EP003-IL2-IgG1-Fc, T3A, C125S; Fc Knob mutation: S354C, T366W, K409A; ADCC mutation: L234A, L235A, P329G
APASSSTKKTQLQLEHLLLDLQMILNGINNYKNPKLTRMLTAKFYMPKKATELKHLQCLEEELKPLEEVLNLAQSKNFHLGVTDSISNAIVLARELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 145 - protein, 360 aa, Artificial Sequence
  Synthetic sequence EP297 (EP001-IL2-IgG1-Fc, T3A, C125S; Fc Knob mutation: S354C, T366W, K409A; ADCC mutation: L234A, L235A, P329G)
APASSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 146 - protein, 141 aa, Artificial Sequence
  Synthetic sequence EP321 (WT-IL2 with C125S mutation)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSHHHHHH

SEQ ID NO: 147 - protein, 141 aa, Artificial Sequence
  Synthetic sequence EP329
APTSSSTKKTQLQLEHLLLDLQMILNGINNYKNPGLTEMLTFKFYMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 148 - protein, 141 aa, Artificial Sequence
  Synthetic sequence EP330
APTSSSTKKTQLQLEHLLLDLQMILNGINNYKNPGLTEMLTFKFYMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 149 - protein, 141 aa, Artificial Sequence
  Synthetic sequence EP331
APTSSSTKKTQLQLEHLLLDLQMILNGINNYKNPGLTEMLTFKFYMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 150 - protein, 141 aa, Artificial Sequence
  Synthetic sequence EP332
APTSSSTKKTQLQLEHLLLDLQMILNGINNYKNPGLTEMLTFKFYMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 151 - protein, 141 aa, Artificial Sequence
  Synthetic sequence EP333
APTSSSTKKTQLQLEHLLLDLQMILNGINNYKNPSLTRMLTGKFYMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 152 - protein, 141 aa, Artificial Sequence
  Synthetic sequence EP334
APTSSSTKKTQLQLEHLLLDLQMILNGINNYKNPSLTRMLTGKFYMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 153 - protein, 141 aa, Artificial Sequence
  Synthetic sequence EP335
APTSSSTKKTQLQLEHLLLDLQMILNGINNYKNPSLTRMLTGKFYMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 154 - protein, 141 aa, Artificial Sequence
  Synthetic sequence EP336
APTSSSTKKTQLQLEHLLLDLQMILNGINNYKNPSLTRMLTGKFYMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 155 - protein, 141 aa, Artificial Sequence
  Synthetic sequence EP337
APTSSSTKKTQLQLEHLLLDLQMILNGINNYKNPLLTDMLTRKFYMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 156 - protein, 141 aa, Artificial Sequence
  Synthetic sequence EP338
APTSSSTKKTQLQLEHLLLDLQMILNGINNYKNPLLTDMLTRKFYMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 157 - protein, 141 aa, Artificial Sequence
  Synthetic sequence EP339
APTSSSTKKTQLQLEHLLLDLQMILNGINNYKNPLLTDMLTRKFYMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 158 - protein, 141 aa, Artificial Sequence
  Synthetic sequence EP340
APTSSSTKKTQLQLEHLLLDLQMILNGINNYKNPLLTDMLTRKFYMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 159 - protein, 141 aa, Artificial Sequence
  Synthetic sequence EP341
APTSSSTKKTQLQLEHLLLDLQMILNGINNYKNPKLTDMLTFKFSMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 160 - protein, 141 aa, Artificial Sequence
  Synthetic sequence EP342
APTSSSTKKTQLQLEHLLLDLQMILNGINNYKNPKLTDMLTFKFSMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 161 - protein, 141 aa, Artificial Sequence
  Synthetic sequence EP343
APTSSSTKKTQLQLEHLLLDLQMILNGINNYKNPKLTDMLTFKFSMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 162 - protein, 141 aa, Artificial Sequence
  Synthetic sequence EP344
APTSSSTKKTQLQLEHLLLDLQMILNGINNYKNPKLTDMLTFKFSMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 163 - protein, 141 aa, Artificial Sequence
  Synthetic sequence EP345
APTSSSTKKTQLQLEHLLLDLQMILNGINNYKNPKLTVMLTFKFSMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 164 - protein, 141 aa, Artificial Sequence
  Synthetic sequence EP346
APTSSSTKKTQLQLEHLLLDLQMILNGINNYKNPKLTVMLTFKFSMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 165 - protein, 141 aa, Artificial Sequence
  Synthetic sequence EP347
APTSSSTKKTQLQLEHLLLDLQMILNGINNYKNPKLTVMLTFKFSMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 166 - protein, 141 aa, Artificial Sequence
  Synthetic sequence EP348
APTSSSTKKTQLQLEHLLLDLQMILNGINNYKNPKLTVMLTFKFSMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 167 - protein, 141 aa, Artificial Sequence
  Synthetic sequence EP349
APTSSSTKKTQLQLEHLLLDLQMILNGINNYKNPKLTRMLTAKFYMPKKATELKHLQCLEEELKPLEEVLNLAQSKNFHLTARDAIDNINVIVQELKGSETTFMCEYADETATIVEFLNRWITFSQSIISTLTGSHHHHHH

SEQ ID NO: 168 - protein, 141 aa, Artificial Sequence
  Synthetic sequence EP350
APTSSSTKKTQLQLEHLLLDLQMILNGINNYKNPKLTRMLTAKFYMPKKATELKHLQCLEEELKPLEEVLNLAQSKNFHLTARDAISNINVIIQELKGSETTFMCEYADETATIVEFLNRWITFSQSIISTLTGSHHHHHH

SEQ ID NO: 169 - protein, 141 aa, Artificial Sequence
  Synthetic sequence EP351
APTSSSTKKTQLQLEHLLLDLQMILNGINNYKNPKLTRMLTAKFYMPKKATELKHLQCLEEELKPLEEVLNLAQSKNFHLTPRDAIDNIRVIVQELKGSETTFMCEYADETATIVEFLNRWITFSQSIISTLTGSHHHHHH

SEQ ID NO: 170 - protein, 141 aa, Artificial Sequence
  Synthetic sequence EP352
APTSSSTKKTQLQLEHLLLDLQMILNGINNYKNPKLTRMLTAKFYMPKKATELKHLQCLEEELKPLEEVLNLAQSKNFHLTPRDAIDNIRVIILELKGSETTFMCEYADETATIVEFLNRWITFSQSIISTLTGSHHHHHH

SEQ ID NO: 171 - DNA, 66 bp, Artificial Sequence
  Synthetic sequence
  misc_feature (16)..(17): n = A,T,C or G
  misc_feature (18)..(18): n is G or C
  misc_feature (25)..(26): n = A,T,C or G
  misc_feature (27)..(27): n is G or C
aactacaagaaccccnnnctgaccnnnatgctgaccttcaagttctacatgcctaagaaggccacc

SEQ ID NO: 172 - DNA, 66 bp, Artificial Sequence
  Synthetic sequence
  misc_feature (16)..(17): n = A,T,C or G
  misc_feature (18)..(18): n is G or C
  misc_feature (37)..(38): n = A,T,C or G
  misc_feature (39)..(39): n is G or C
aactacaagaaccccnnnctgacccggatgctgaccnnnaagttctacatgcctaagaaggccacc

SEQ ID NO: 173 - DNA, 66 bp, Artificial Sequence
  Synthetic sequence
  misc_feature (16)..(17): n = A,T,C or G
  misc_feature (18)..(18): n is G or C
  misc_feature (46)..(47): n = A,T,C or G
  misc_feature (48)..(48): n is G or C
aactacaagaaccccnnnctgacccggatgctgaccttcaagttcnnnatgcctaagaaggccacc

SEQ ID NO: 174 - DNA, 66 bp, Artificial Sequence
  Synthetic sequence
  misc_feature (25)..(26): n = A,T,C or G
  misc_feature (27)..(27): n is G or C
  misc_feature (37)..(38): n = A,T,C or G
  misc_feature (39)..(39): n is G or C
aactacaagaaccccaagctgaccnnnatgctgaccnnnaagttctacatgcctaagaaggccacc

SEQ ID NO: 175 - DNA, 66 bp, Artificial Sequence
  Synthetic sequence
  misc_feature (25)..(26): n = A,T,C or G
  misc_feature (27)..(27): n is G or C
  misc_feature (46)..(47): n = A,T,C or G
  misc_feature (48)..(48): n is G or C
aactacaagaaccccaagctgaccnnnatgctgaccttcaagttcnnnatgcctaagaaggccacc

SEQ ID NO: 176 - DNA, 66 bp, Artificial Sequence
  Synthetic sequence
  misc_feature (37)..(38): n = A,T,C or G
  misc_feature (39)..(39): n is G or C
  misc_feature (46)..(47): n = A,T,C or G
  misc_feature (48)..(48): n is G or C
aactacaagaaccccaagctgacccggatgctgaccnnnaagttcnnnatgcctaagaaggccacc

SEQ ID NO: 177 - DNA, 66 bp, Artificial Sequence
  Synthetic sequence
  misc_feature (16)..(17): n = A,T,C or G
  misc_feature (18)..(18): n is G or C
  misc_feature (25)..(26): n = A,T,C or G
  misc_feature (27)..(27): n is G or C
  misc_feature (37)..(38): n = A,T,C or G
  misc_feature (39)..(39): n is G or C
aactacaagaaccccnnnctgaccnnnatgctgaccnnnaagttctacatgcctaagaaggccacc

SEQ ID NO: 178 - DNA, 66 bp, Artificial Sequence
  Synthetic sequence
  misc_feature (16)..(17): n = A,T,C or G
  misc_feature (18)..(18): n is G or C
  misc_feature (37)..(38): n = A,T,C or G
  misc_feature (39)..(39): n is G or C
  misc_feature (46)..(47): n = A,T,C or G
  misc_feature (48)..(48): n is G or C
aactacaagaaccccnnnctgacccggatgctgaccnnnaagttcnnnatgcctaagaaggccacc

SEQ ID NO: 179 - DNA, 66 bp, Artificial Sequence
  Synthetic sequence
  misc_feature (25)..(26): n = A,T,C or G
  misc_feature (27)..(27): n is G or C
  misc_feature (37)..(38): n = A,T,C or G
  misc_feature (39)..(39): n is G or C
  misc_feature (46)..(47): n = A,T,C or G
  misc_feature (48)..(48): n is G or C
aactacaagaaccccaagctgaccnnnatgctgaccnnnaagttcnnnatgcctaagaaggccacc

SEQ ID NO: 180 - DNA, 66 bp, Artificial Sequence
  Synthetic sequence
aactacaagaaccccaagctgacccggatgctgaccgccaagttctacatgcctaagaaggccacc

SEQ ID NO: 181 - DNA, 66 bp, Artificial Sequence; identical to SEQ ID NO: 180
  Synthetic sequence

SEQ ID NO: 182 - protein, 12 aa, Artificial Sequence
  Synthetic sequence
PKLTRMLTFKFY

SEQ ID NO: 183 - protein, 12 aa, Artificial Sequence
  Synthetic sequence
PVLTRMLTIKFY

SEQ ID NO: 184 - protein, 12 aa, Artificial Sequence
  Synthetic sequence
PKLTRMLTLKFP

SEQ ID NO: 185 - protein, 12 aa, Artificial Sequence
  Synthetic sequence
PDLTSMLAFKFY

SEQ ID NO: 186 - protein, 12 aa, Artificial Sequence
  Synthetic sequence
PGLTEMLTFKFY

SEQ ID NO: 187 - protein, 12 aa, Artificial Sequence
  Synthetic sequence
PSLTRMLTGKFY

SEQ ID NO: 188 - protein, 12 aa, Artificial Sequence
  Synthetic sequence
PELTIMLTPKFY

SEQ ID NO: 189 - protein, 12 aa, Artificial Sequence
  Synthetic sequence
PCLTAMLTLKFA

SEQ ID NO: 190 - protein, 12 aa, Artificial Sequence
  Synthetic sequence
PCLTAMLTLKFA

SEQ ID NO: 191 - protein, 12 aa, Artificial Sequence
  Synthetic sequence
PKLTRMLTHKFV

SEQ ID NO: 192 - protein, 12 aa, Artificial Sequence
  Synthetic sequence
PCLTDMLTFKFY

SEQ ID NO: 193 - protein, 12 aa, Artificial Sequence
  Synthetic sequence
PLLTDMLTRKFY

SEQ ID NO: 194 - protein, 12 aa, Artificial Sequence
  Synthetic sequence
PLLTDMLTFKFY

SEQ ID NO: 195 - protein, 12 aa, Artificial Sequence
  Synthetic sequence
PKLTDMLTFKFS

SEQ ID NO: 196 - protein, 12 aa, Artificial Sequence
  Synthetic sequence
PKLTYMLTRKFY

SEQ ID NO: 197 - protein, 12 aa, Artificial Sequence
  Synthetic sequence
PKLTRMLTFKFC

SEQ ID NO: 198 - protein, 12 aa, Artificial Sequence
  Synthetic sequence
PKLTSMLTFKFS

SEQ ID NO: 199 - protein, 12 aa, Artificial Sequence
  Synthetic sequence
PKLTSMLTFKFS

SEQ ID NO: 200 - protein, 12 aa, Artificial Sequence
  Synthetic sequence
PKLTYMLTFKFS

SEQ ID NO: 201 - protein, 12 aa, Artificial Sequence
  Synthetic sequence
PKLTYMLTFKFS

SEQ ID NO: 202 - protein, 12 aa, Artificial Sequence
  Synthetic sequence
PKLTGMLTFKFS

SEQ ID NO: 203 - protein, 12 aa, Artificial Sequence
  Synthetic sequence
PKLTVMLTFKFT

SEQ ID NO: 204 - protein, 12 aa, Artificial Sequence
  Synthetic sequence
PKLTVMLTFKFS

SEQ ID NO: 205 - protein, 12 aa, Artificial Sequence
  Synthetic sequence
PKLTVMLTFKFP

SEQ ID NO: 206 - protein, 12 aa, Artificial Sequence
  Synthetic sequence
PKLTVMLTFKFF

SEQ ID NO: 207 - protein, 12 aa, Artificial Sequence
  Synthetic sequence
PKLTCMLTFKFA

SEQ ID NO: 208 - protein, 12 aa, Artificial Sequence
  Synthetic sequence
PKLTNMLTFKFA

SEQ ID NO: 209 - protein, 12 aa, Artificial Sequence
  Synthetic sequence
PKLTNMLTFKFS

SEQ ID NO: 210 - protein, 15 aa, Artificial Sequence
  Synthetic sequence EP242 beta region 2 motif
TARDAISNIRVIVQE

SEQ ID NO: 211 - protein, 15 aa, Artificial Sequence
  Synthetic sequence EP247 beta region 2
RARDAIDNIRVIVQE

SEQ ID NO: 212 - protein, 15 aa, Artificial Sequence
  Synthetic sequence EP251 beta region 2
TPRDAIDNINVIIQE

SEQ ID NO: 213 - protein, 15 aa, Artificial Sequence
  Synthetic sequence EP252 beta region 2
TPRDAIDNIRVIVQE

SEQ ID NO: 214 - protein, 15 aa, Artificial Sequence
  Synthetic sequence EP253 beta region 2
TPRDAIDNIRVIILE

SEQ ID NO: 215 - protein, 15 aa, Artificial Sequence
  Synthetic sequence EP258 beta region 2
TARDAISNINVIIQE

SEQ ID NO: 216 - protein, 15 aa, Artificial Sequence
  Synthetic sequence EP260 beta region 2
TARDAIDNINVIVQE

SEQ ID NO: 217 - protein, 15 aa, Artificial Sequence
  Synthetic sequence EP263 beta region 2
TARDAIDNIRVIVLE

SEQ ID NO: 218 - protein, 5 aa, Artificial Sequence
  Synthetic sequence
GSGGS

SEQ ID NO: 219 - protein, 5 aa, Artificial Sequence
  Synthetic sequence
GGGGS

SEQ ID NO: 220 - protein, 4 aa, Artificial Sequence
  Synthetic sequence
GGGS

(19) United States
(12) Patent Application Publication
(10) Pub. No.: US 2023/0002466 A1
(43) Pub. Date: Jan. 5, 2023

(54) ENGINEERED INTERLEUKIN-2 RECEPTOR BETA AGONISTS

(71) Applicant: Elpis Biopharmaceuticals, Lexington, MA (US)

(72) Inventors: Yan CHEN, Lexington, MA (US); Kehao ZHAO, Lexington, MA (US); Christina SWANSON, Lexington, MA (US); Jenna NGUYEN, Lexington, MA (US); Nathan KALLEN, Lexington, MA (US); Samuel Clement HASSAN, Lexington, MA (US); Ning JIANG, Lexington, MA (US)

(21) Appl. No.: 17/634,688

(22) PCT Filed: Aug. 13, 2020
(86) PCT No.: PCT/US2020/046244

§ 371 Date: Feb. 11, 2022

Related U.S. Application Data
(60) Provisional application No. 62/886,148, filed on Aug. 13, 2019.

(51) Int. Cl. C07K 14/55; C07K 14/715; A61P 35/00

(52) U.S. Cl. CPC C07K 14/55; C07K 14/7155; A61P 35/00; C07K 2317/55; C07K 2317/622; C07K 2317/31

(57) ABSTRACT

Provided herein are engineered IL2 polypeptides and fusion proteins thereof. Also provided are methods of modulating an immune response by administering an engineered IL2 polypeptide or a fusion protein thereof. The engineered IL2 polypeptides and fusion proteins thereof demonstrate increased binding to IL2Rβ, decreased binding to IL2Rα, or both.

### CROSS-REFERENCE TO RELATED APPLICATIONS

This application claims the benefit of priority to U.S. Provisional Application No. 62/886,148, filed Aug. 13, 2019, which is hereby incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 300096_401WO_SEQUENCE_LISTING.txt. The text file is 230 KB, was created on Aug. 13, 2020, and is being submitted electronically via EFS-Web.

### BACKGROUND

Interleukin-2 (IL2) is a cytokine that modulates lymphocyte proliferation and activation. It has a length of 133 amino acids and the structure includes four antiparallel, amphipathic C-helices. IL2 mediates its action by binding to IL2 receptors (IL2R), which includes up to three individual subunits. Association of all three subunits, the interleukin-2 receptor alpha chain (IL2Rα, or CD25), interleukin-2 receptor beta chain (IL2Rβ, or CD122), and interleukin-2 receptor gamma chain (IL2Rγ, or CD132), results in a trimeric IL2Rαβγ, which is a high-affinity receptor for IL2. Association of the IL2Rβ and IL2Rγ subunits results in the dimeric receptor IL2Rβγ, and is termed an intermediate affinity IL2R. The IL2Rα subunit forms a monomeric low affinity IL2 receptor. Expression of IL2Rα is involved in the expansion of immunosuppressive regulatory T cells (Tregs); whereas dimeric IL2Rβγ can result in cytolytic CD8⁺ T cell and NK cell proliferation and killing in the absence of IL2Rα.

### BRIEF SUMMARY

The present disclosure provides engineered IL2 polypeptides having improved binding to IL2Rβ as compared to wild-type IL2 and/or reduced binding to IL2Rα as compared to wild-type IL2.

In one aspect, the present disclosure provides an engineered interleukin-2 (IL2) polypeptide comprising an engineered IL2 receptor β (IL2Rβ) binding region 2 comprising: X₁-X₂- X₃-D-X₄-X-₅-X₆-N-X₇-X₈-X₉-X₁₀-X₁₁-X₁₂-X₁₃ (SEQ ID NO: 1),

wherein X₁, X₃, X₆, X₈, X₁₂, and X₁₃ each comprise any residue,

wherein X₂, X₄, and X₁₀ are uncharged residues,

wherein X₅, X₇, X₉, and X₁₁ each comprise uncharged, nonpolar residues, and

wherein the engineered IL2 polypeptide binds to IL2Rβ at a K_{D} at least 10-fold greater than a wild-type IL2.

In certain aspects, X₁ is an uncharged polar residue, an uncharged nonpolar residue, a basic residue, or an acidic residue; X₁ is selected from C, T, G, W, I, S, E, and K; or X₁ is selected from G, K, E, C, and T. In certain aspects, X₂ is an uncharged polar residue or an uncharged nonpolar residue; X₂ is selected from Y, P, V, W, L, A, and G; or X₂ is selected from V, P, W, and A. In certain aspects, X₃ is an uncharged polar residue, an uncharged nonpolar residue, a basic residue, or an acidic residue; X₃ is selected from S, T, Q, G, M, E, R, and K; or X₃ is selected from T, G, S, R, and E. In certain aspects, X₄ is not L; X₄ is an uncharged nonpolar residue or an uncharged polar residue; or X₄ is selected from A, V, S, and T. In certain aspects, X₅ is selected from I, L, T, and V; or X₅ is selected from I and V. In certain aspects, X₆ is an uncharged polar residue, a basic residue, or an acidic residue; X₆ is selected from S, T, E, D, and R; or X₆ is selected from S, D, E, and T. In certain aspects, X₇ is selected from I, A, M, and V; or X₇ is selected from I, A, and M. In certain aspects, X₈ is an uncharged polar residue, an uncharged nonpolar residue, a basic residue, or an acidic residue; X₈ is selected from S, T, N, Q, I, G, E, K, and R; or X₈ is selected from I, R, N, and T. In certain aspects, X₉ is selected from V, L, and I; or X₉ is V. In certain aspects, X₁₀ is an uncharged polar residue or an uncharged nonpolar residue; X₁₀ is selected from N, T, I, and L; or X₁₀ is selected from I and L. In certain aspects, X₁₁ is a uncharged nonpolar residue; or X₁₁ is selected from V, A, and I. In certain embodiments, X₁₂ is an uncharged polar residue, an uncharged nonpolar residue, or an acidic residue; X₁₂ is selected from Q, L, G, K, and R; or X₁₂ is selected from R, G, Q, and K. In certain aspects, X₁₃ is an uncharged nonpolar residue or a basic residue; X₁₃ is selected from A, D, and E; or X₁₃ is selected from E and A.

In some aspects, the present disclosure provides an engineered IL2 polypeptide comprising a substitution to at least one residue selected from: R81, P82, R83, L85, I86, S87, I89, N90, I92, V93, and L94. In certain aspects, the R81 substitution is selected from R81G, R81K, R81E, R81C, and R81T; the R83 substitution is selected from R83T, R83G, R83S, and R83E; the L85 substitution is selected from L85S, L85A, L85V, and L85T; the I92 substitution is I92L; and the L94 substitution is selected from L94R, L94G, L94Q, and L94K. In certain aspects, the engineered IL2 polypeptide comprises substitutions to R81 and L83. In certain aspects, the engineered IL2 polypeptide comprises substitutions to R81, L83, S87, N90, and N94; substitutions to R81, L83, S87, N90, and V93; substitutions to R81, L83, P82, and V93; or substitutions to R81, L83, and N90.

In one aspect, the present disclosure provides an engineered interleukin-2 (IL2) polypeptide comprising an engineered IL2 receptor α (IL2Rα) binding region 1 motif. comprising a substitution selected from: a substitution at position K35, a substitution at R38, a substitution at F42, a substitution at Y45, or combinations thereof, wherein the engineered IL2 polypeptide binds to IL2Rα with at least 2-fold reduced binding kinetics as compared to wild-type IL2.

In some aspects, the present disclosure provides an engineered IL2 polypeptide including an engineered IL2 receptor β (IL2Rβ) binding region 2 as previously described and an engineered IL2 receptor a (IL2Rα) binding region 1 as previously described.

In some aspects, the present disclosure provides an engineered IL2 polypeptide as provided herein, fused to a half-life extending molecule.

In some aspects, the present disclosure provides a fusion polypeptide comprising a first polypeptide and a second polypeptide, wherein the first polypeptide comprises an engineered IL2 polypeptide as provided herein.

In some aspects, the present disclosure provides an isolated polynucleotide encoding an engineered IL2 polypeptide or a fusion polypeptide thereof, an expression vector comprising the isolated polynucleotide, or a modified cell comprising the isolated polynucleotide or expression vector.

In some aspects, the present disclosure provides a pharmaceutical composition comprising the engineered IL2 polypeptide or fusion polypeptide thereof, and a pharmaceutically acceptable carrier.

In some aspects, the present disclosure provides a method of modulating an immune response in a subject in need thereof, the method comprising administering to the subject a therapeutically effective amount of an engineered IL2 polypeptide or fusion polypeptide thereof, or a pharmaceutical composition thereof. In certain aspects, modulating the immune response comprises at least one of: enhancing effector T cell activity, enhancing NK cell activity, and suppressing regulatory T cell activity.

In some aspects, the present disclosure provides a method of treating a disease in a subject in need thereof, the method comprising administering to the subject a therapeutically effective amount of an engineered IL2 polypeptide or fusion polypeptide thereof, or a pharmaceutical composition thereof. In certain aspects, the disease is cancer. In certain aspects, the method further comprises administering an additional therapeutic agent, such as an antigen binding moiety, an immune cell expressing a chimeric antigen receptor, an immune cells expressing an engineered T cell receptor, a tumor infiltrating lymphocyte, an immune checkpoint inhibitor, an oncolytic virus, a tumor microenvironment (TME) inhibitor, or a cancer vaccine. In certain aspects, the methods comprise administering to the subject an immune cell comprising a polynucleotide encoding the engineered IL2 polypeptide or fusion polypeptide thereof.

### BRIEF DESCRIPTION OF THE SEVERAL VIEWS OF THE DRAWINGS

FIG. 1A shows the regions of IL2 responsible for binding IL2Rα (solid open boxes), IL2Rβ (dashed boxes), and IL2Rγ (gray boxes), FIG. 1B shows a graphic depiction of IL2Rα, IL2Rβ, and IL2Rγ bound to IL2, and FIG. 1C shows the IL2Rα binding site of IL2 highlighting four residues (K35, R38, F42 and Y45) of IL2 critical for IL2Rα interaction.

FIGS. 2A-2B show the identification of IL2Rα reduced binding mutations by ELISA.

FIG. 3 shows characterization of IL2Rα-reduced binding mutations by surface plasmon resonance sequence alignments of IL2Rα reduced binding mutations.

FIG. 4 shows characterization of IL2Rα-reduced binding mutations by surface plasmon resonance.

FIG. 5 shows the IL2Rβ agonist mutagenic libraries FIG. 6 shows IL2Rβ agonist expression in E. coli and binding to IL2Rβ.

FIG. 7 shows a multiple sequence alignment of the IL2Rβ binding region 2 for IL2Rβ agonists identified through mRNA display.

FIG. 8 shows SDS analysis of IL2Rβ agonist clones produced in E. coli.

FIGS. 9A-9B show sensorgrams and binding kinetics of wild-type IL2 to IL2Rα (FIG. 9A) and IL2Rβ (FIG. 9B) in SPR.

FIGS. 10A-10H show sensorgrams of E. coli produced wild-type IL2 and engineered IL2Rβ agonists to IL2Rα in SPR. FIG. 10A shows a sensorgram of wild-type

IL2; FIG. 10B shows a sensorgram of EP001; FIG. 10C shows a sensorgram of EP004; FIG. 10D shows a sensorgram of EP005; FIG. 10E shows a sensorgram of EP002; FIG. 10F shows a sensorgram of EP03; FIG. 10G shows a sensorgram of EPIM-06; and FIG. 10H shows a sensorgram of EP007.

FIGS. 11A-11H show sensorgrams of E. coli produced wild-type IL2 and engineered IL2Rβ agonists to IL2Rβ in SPR. FIG. 11A shows a sensorgram of EP003; FIG. 11B shows a sensorgram of EP005; FIG. 11C shows a sensorgram of E002; FIG. 11D shows a sensorgram of EP001; FIG. 11E shows a sensorgram of EP007; FIG. 11F shows a sensorgram of EP006; FIG. 11G shows a sensorgram of EP004; and FIG. 11H shows a sensorgram of wild-type IL2.

FIGS. 12A-12D show sensorgrams of IL2Rβ binding to mammalian produced wild-type IL2 (FIG. 12A) and engineered IL2Rβ agonists EP0001 (FIG. 12B), EP0003 (FIG. 12C), and EP004 (FIG. 12D) in SPR.

FIGS. 13A-13D show sensorgrams of IL2Rα binding to mammalian produced wild-type IL2 (FIG. 13A) and engineered IL2Rβ agonists EP001 (FIG. 13B), EP003 (FIG. 13C), and EP004 (FIG. 13D) in SPR.

FIGS. 14A-14I show pSTAT5 expression as measured from blood donors 1-3, following stimulation of human PBMCs with wild-type IL2 and engineered IL2Rβ agonists, as measured for CD8+ T cells (FIG. 14A for blood donor 1, FIG. 14D for blood donor 2, and FIG. 14G for blood donor 3), NK cells (FIG. 14B for blood donor 1, FIG. 14E for blood donor 2, and FIG. 14H for blood donor 3), and T regs (FIG. 14C for blood donor 1, FIG. 14F for blood donor 2, and FIG. 14I for blood donor 3).

FIGS. 15A-15B show characterization by ELISA of IL2Rβ agonist EP001 back-mutation clones for binding to IL2Rα (FIG. 15A) and IL2Rβ (FIG. 15B) by ELISA.

FIGS. 16A-16H show IL2Rβ binding sensorgrams of IL2Rβ agonist EP001 back-mutation clones by SPR.

FIG. 17 shows examples of SDS-PAGE results for purified clones of IL2Rα/IL2Rβ clones.

FIGS. 18A-18H show binding sensorgrams of engineered IL2Rα/IL2Rβ clones to human IL2a by SPR.

FIGS. 19A-19H show binding sensorgrams of engineered IL2Rα/IL2Rβ clones to human IL2β by SPR.

FIGS. 20A-20G show single concentration binding sensorgrams of engineered IL2Rα/IL2Rβ clones to human IL2Rα by SPR.

FIGS. 21A-21G show single concentration binding of engineered IL2Rα/IL2Rβ clones to human IL2Rβ by SPR.

FIGS. 22A-22B show multi-concentration binding of engineered IL2Rα/IL2Rβ clones to human IL2Rα (FIG. 22A) and IL2Rβ (FIG. 22B) by SPR.

FIGS. 23A-23E show ELISA binding to human IL2Rα. FIG. 23A shows the ELISA binding of EP252 and its IL2Rα binding reduced mutations to human IL2Rα. FIG. 23B shows the ELISA binding of EP253 and its IL2Rα binding reduced mutations to human IL2Rα. FIG. 23C shows the ELISA binding of EP258 and its IL2Rα binding reduced mutations to human IL2Rα. FIG. 23D shows the ELISA binding of EP260 and its IL2Rα binding reduced mutations to human IL2Rα. FIG. 23E shows dose dependent binding for selected engineered IL2Rβ/IL2Rα clones.

FIGS. 24A-24D show ELISA binding to human IL2Rβ. FIG. 24A shows the ELISA binding of EP252 and its IL2Rα binding reduced mutations to human IL2Rβ. FIG. 24B shows the ELISA binding of EP253 and its IL2Rα binding reduced mutations to human IL2Rβ. FIG. 24C shows the ELISA binding of EP258 and its IL2Rα binding reduced mutations to human IL2Rβ. FIG. 24D shows the ELISA binding of EP260 and its IL2Rα binding reduced mutations to human IL2Rβ.

FIGS. 25A-25D show p-STAT5 activation of human CD8+ T cells from donor 656, by engineered IL2Rα/IL2Rβ clones. FIG. 25A shows p-STAT5 activation of human CD8+ T cells for EP252 and its IL2Rα binding reduced mutations. FIG. 25B shows p-STAT5 activation of human CD8+ T cells for EP253 and its IL2Rα binding reduced mutations. FIG. 25C shows p-STAT5 activation of human CD8+ T cells for EP258 and its IL2Rα binding reduced mutations. FIG. 25D shows p-STAT5 activation of human CD8+ T cells for EP260 and its IL2Rα binding reduced mutations.

FIGS. 26A-26D show p-STAT5 activation of human CD8+ T cells from donor 648, by engineered IL2Rα/IL2Rβ clones. FIG. 26A shows p-STAT5 activation of human CD8+ T cells for EP252 and its IL2Rα binding reduced mutations. FIG. 26B shows p-STAT5 activation of human CD8+ T cells for EP253 and its IL2Rα binding reduced mutations. FIG. 26C shows p-STAT5 activation of human CD8+ T cells for EP258 and its IL2Rα binding reduced mutations. FIG. 26D shows p-STAT5 activation of human CD8+ T cells for EP260 and its IL2Rα binding reduced mutations.

FIGS. 27A-27D show p-STAT5 activation of human NK cells from donor 656, by engineered IL2Rα/IL2Rβ clones. FIG. 27A shows p-STAT5 activation of human NK cells for EP252 and its IL2Rα binding reduced mutations. FIG. 25B shows p-STAT5 activation of human NK cells for EP253 and its IL2Rα binding reduced mutations. FIG. 27C shows p-STAT5 activation of human NK cells for EP258 and its IL2Rα binding reduced mutations. FIG. 27D shows p-STAT5 activation of human NK cells for EP260 and its IL2Rα binding reduced mutations.

FIGS. 28A-28D show p-STAT5 activation of human NK cells from donor 648, by engineered IL2Rα/IL2Rβ clones. FIG. 28A shows p-STAT5 activation of human NK cells for EP252 and its IL2Rα binding reduced mutations. FIG. 28B shows p-STAT5 activation of human NK cells for EP253 and its IL2Rα binding reduced mutations. FIG. 28C shows p-STAT5 activation of human NK cells for EP258 and its IL2Rα binding reduced mutations. FIG. 28D shows p-STAT5 activation of human NK cells for EP260 and its IL2Rα binding reduced mutations.

FIGS. 29A-29D show p-STAT5 activation of human T reg cells from donor 656, by engineered IL2Rα/IL2Rβ clones. FIG. 29A shows p-STAT5 activation of human T reg cells for EP252 and its IL2Rα binding reduced mutations. FIG. 29B shows p-STAT5 activation of human T reg cells for EP253 and its IL2Rα binding reduced mutations. FIG. 29C shows p-STAT5 activation of human T reg cells for EP258 and its IL2Rα binding reduced mutations. FIG. 29D shows p-STAT5 activation of human T reg cells for EP260 and its IL2Rα binding reduced mutations.

FIGS. 30A-30D show p-STAT5 activation of human T reg cells from donor 648, by engineered IL2Rα/IL2Rβ clones. FIG. 30A shows p-STAT5 activation of human T reg cells for EP252 and its IL2Rα binding reduced mutations. FIG. 30B shows p-STAT5 activation of human T reg cells for EP253 and its IL2Rα binding reduced mutations. FIG. 30C shows p-STAT5 activation of human T reg cells for EP258 and its IL2Rα binding reduced mutations. FIG. 30D shows p-STAT5 activation of human T reg cells for EP260 and its IL2Rα binding reduced mutations.

FIGS. 31A-31D show p-STAT5 activation of murine CD8+ T cells. FIG. 31A shows p-STAT5 activation of murine CD8+ T cells for EP252 and its IL2Rα binding reduced mutations. FIG. 31B shows p-STAT5 activation of murine CD8+ T cells for EP253 and its IL2Rα binding reduced mutations. FIG. 31C shows p-STAT5 activation of murine CD8+ T cells for EP258 and its IL2Rα binding reduced mutations. FIG. 31D shows p-STAT5 activation of murine CD8+ T cells for EP260 and its IL2Rα binding reduced mutations.

FIGS. 32A-32D show p-STAT5 activation of murine NK cells. FIG. 32A shows p-STAT5 activation of murine NK cells for EP252 and its IL2Rα binding reduced mutations. FIG. 32B shows p-STAT5 activation of murine NK cells for EP253 and its IL2Rα binding reduced mutations. FIG. 32C shows p-STAT5 activation of murine NK cells for EP258 and its IL2Rα binding reduced mutations. FIG. 32D shows p-STAT5 activation of murine NK cells for EP260 and its IL2Rα binding reduced mutations.

FIGS. 33A-33D show p-STAT5 activation of murine T regulatory cells. FIG. 33A shows p-STAT5 activation of murine T regulatory cells for EP252 and its IL2Rα binding reduced mutations. FIG. 33B shows p-STAT5 activation of murine T regulatory cells for EP253 and its IL2Rα binding reduced mutations. FIG. 33C shows p-STAT5 activation of murine T regulatory cells for EP258 and its IL2Rα binding reduced mutations. FIG. 33D shows p-STAT5 activation of murine T regulatory cells for EP260 and its IL2Rα binding reduced mutations.

FIG. 34 shows a summary of p-STAT5 activation of murine CD8+ T cells, NK cells, and Tregs.

FIGS. 35A-35C show structure diagrams of monovalent and bivalent IL2Rβ agonist Fc fusion proteins.

FIGS. 36A-36B show SDS-PAGE analysis of the purified IL2Rβ agonist Fc fusion proteins.

FIGS. 37A-37G show receptor binding analysis by ELISA of bivalent IL2Rβ agonist Fc fusion proteins.

FIGS. 38A-38B show receptor binding analysis by ELISA of monovalent IL2Rβ agonist Fc fusion proteins.

FIGS. 39A-39D show receptor binding analysis of monovalent IL2Rβ Fc fusion proteins by SPR.

FIGS. 40A-40C show p-STAT5 activation of human PBMCs by bivalent IL2Rβ agonist Fc-fusion proteins.

FIGS. 41A-41C show p-STAT5 activation of human PBMCs by monovalent IL2Rβ agonist Fc-fusion proteins.

FIGS. 42A-42B show pharmacokinetics with murine i.v. (FIG. 42A) and i.p. (FIG. 42B) administration of IL2Rβ agonist.

FIGS. 43A-43D show normalized counts of tumor infiltrating immune cells following administration of IL2Rβ agonist.

FIGS. 44A-44B show ratios of effector cells to T regulatory cell in tumors.

FIGS. 45A-45C show percentages of effector and memory T cells.

### DETAILED DESCRIPTION

IL2 has been a promising new immunotherapy, but therapies based on wild-type human IL2 may activate T regulatory cells in addition to activating effector T cells and NK cells. Activation of T regulatory cells by IL2 may hamper the anti-cancer response that may otherwise be elicited by IL2. Thus, IL2-based therapies with reduced activation of T regulatory cells and/or preferential activation of T effector cells, NK cells, or a combination thereof are needed.

Presented herein are rationally designed IL2Rβ agonists, which are engineered IL2 polypeptides having amino acid substitutions in IL2Rβ binding region 2 that enhance binding to IL2Rβ. The engineered IL2Rβ agonists provide the advantage of increasing stimulation of NK cells and T effector cells compared to wild-type IL2, but not T regulatory cells. Thus, the engineered IL2Rβ agonists are useful for modulating or activating an immune response, for example, for treatment of cancer.

The term “interleukin-2 or “IL2” as used herein, refers to an IL2 from any vertebrate source, including mammals such humans or mice, unless otherwise indicated. The term encompasses precursor or unprocessed IL2, as well as any form of IL2 that results from cellular processing. The term also encompasses naturally occurring variants of IL2, such as splice variants or allelic variants. The amino acid sequence of an example mature human IL2 is shown in SEQ ID NO: 65. Precursor or unprocessed human IL2 is shown in SEQ ID NO: 66, and includes a 20-residue signal peptide, which is absent in the mature IL2 polypeptide. “Wild-type” or “native” when used in reference to IL2 is intended to mean the mature IL2 molecule (e.g., SEQ ID NO: 65). The term “engineered IL2” or “engineered IL2 polypeptide” as used herein encompasses an IL2 having at least one residue that differs from a native or wild-type IL2, and includes full-length IL2, truncated forms of IL2, and forms where IL2 is linked or fused with another molecule, such as another polypeptide. The various forms of engineered IL2 are characterized in having at least one amino acid substitution affecting the interaction of IL2 with IL2Rβ and/or IL2Rα. Identification of various engineered forms of IL2 as described herein are made with respect to the sequence shown in, e.g., SEQ ID NO: 22. Various identifiers may be used herein to indicate the same residue substitution. For example, a substitution from arginine at position 81 to threonine can be indicated as R81T or 81T.

IL2Rβ binding region 1 and IL2Rβ binding region 2 are responsible for IL2 binding to IL2Rβ. “IL2Rβ binding region 1” as used herein refers to residues 11-23 of wild-type or native human IL2. The amino acid sequence of IL2Rβ binding region 1 is provided in SEQ ID NO: 67. “IL2Rβ binding region 2” as used herein refers to residues 81-95 of wild-type or native human IL2. The amino acid sequence of IL2Rβ binding region 2 is provided in SEQ ID NO: 68.

IL2Rα binding region 1 and IL2Rα binding region 2 are responsible for IL2 binding to IL2Rα. “IL2Rα binding region 1” as used herein refers to residues 34-45 of wild-type or native human IL2. The amino acid sequence of IL2Rα binding region 1 is provided in SEQ ID NO: 223.

The term “substitution” or “residue substitution” as used herein refers to replacement of a native or wild-type residue with a different residue.

“Any residue” as used herein refers to an amino acid residue having one of the twenty canonical amino acid side chains: alanine (Ala, A); arginine (Arg, R); asparagine (Asn, N); aspartic acid (Asp, D); cysteine (Cys, C); glutamine (Gln, Q); glutamic acid (Glu, E); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

“Uncharged residue” as used herein refers to an amino acid residue with a side chain that does not hold a charge at physiologic pH (pH=7). The uncharged residues are: alanine (Ala, A); asparagine (Asn, N); cysteine (Cys, C); glutamine (Gln, Q); glycine (Gly, G); histidine (His, H); isoleucine (Ile, I); leucine (Leu, L); methionine (Met, M); phenylalanine (Phe, F); proline (Pro, P); serine (Ser, S); threonine (Thr, T); tryptophan (Trp, W); tyrosine (Tyr, Y); and valine (Val, V).

“Uncharged polar residue” as used herein refers to an amino acid residue with a side chain that does not hold a charge at physiologic pH (pH=7) and is hydrophilic. The uncharged polar residues are: asparagine (Asn, N); cysteine (Cys, C); glutamine (Gln, Q); serine (Ser, S); threonine (Thr, T); and tyrosine (Tyr, Y).

“Uncharged nonpolar residue” as used herein refers an amino acid residue with a side chain that does not hold a charge at physiologic pH (pH=7) and is hydrophobic. The uncharged nonpolar residues are: alanine (Ala, A); glycine (Gly, G); histidine (His, H); isoleucine (Ile, I); leucine (Leu, L); methionine (Met, M); phenylalanine (Phe, F); proline (Pro, P); tryptophan (Trp, W); and valine (Val, V).

“Basic residue” as used herein refers to an amino acid residue with a side chain that holds a positive charge at physiologic pH (pH=7). The basic residues are lysine (Lys, K); and arginine (Arg, R).

“Acidic residue” as used herein refers to an amino acid residue with a side chain that holds a negative charge at physiologic pH (pH=7). The acidic residues are aspartic acid (Asp, D); and glutamic acid (Glu, E).

“Fusion polypeptide” or “fusion protein” refers to a polypeptide that is encoded by at least two different DNA sequences corresponding to genes or fragments thereof, which are not naturally expressed from the same gene. An example of a fusion polypeptide is an engineered IL2-Fc fusion polypeptide, which includes an amino acid sequence of an engineered IL2 polypeptide and an amino acid sequence of an Fc domain.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and a ligand). The affinity of a molecule-X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by methods known by persons of skill in the art, including those described herein.

“Half-life extending molecule” as used herein refers to a molecule that when attached (e.g., covalently) to a second molecule, extends the half-life of the second molecule. Examples of half-life extending molecules include an Fc domain, human serum albumin (HSA), an HSA binding molecule, polyethylene glycol (PEG), and polypropylene glycol (PPG).

“Fc domain” or “Fc region” as used herein refers to a polypeptide derived from a C-terminal region of an immunoglobulin heavy chain that contains at least a portion of the constant region. The term includes polypeptides having a native sequence Fc region, or variants thereof. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Examples of Fc regions are disclosed in U.S. Pat. Nos. 7,317,091; 8,735,545; 7,371,826; 7,670,600; and 9,803,023; all of which are incorporated by reference in their entirety.

“Human serum albumin” or “HSA” refers to the serum albumin found in human blood. The commonly used form of HSA has a molecular mass of 66.5 kDa and a half-life of approximately 20 days. Examples of HSA molecules are disclosed in U.S. Pat. Nos. 8,143,026 and 7,189,690, which are incorporated by reference in their entirety.

“HSA binding molecule” refers to a molecule that specifically binds to human serum albumin (HSA), such as an antigen binding moiety having an HSA binding domain.

“Polyethylene glycol” or “PEG,” also referred to as polyethylene oxide or polyoxyethylene is a polyether polymer that may be used to extend half-life.

“Polypropylene glycol” or “PPG,” also referred to as polypropylene oxide, is a polymer of propylene glycol that may be used to extend half-life.

“Antigen binding moiety” refers to the site (i.e., amino acid residues) of an antigen binding molecule (e.g., antibody) that provides interaction with the antigen epitope. An antigen binding moiety may include one or more antibody variable domains (also called antibody variable regions). Preferably, an antigen binding domain comprises an antibody light chain variable region (VL) and an antibody heavy chain variable region (VH). Examples of antigen binding moieties include immunoglobulins, Fab molecules, scFv, bispecific antibodies, diabodies, bi-specific T-cell engagers, and nanobodies. Specific examples of antigen binding moieties include nivolumab, pembrolizumab, pidilizumab, atezolizumab, ipilimumab, tremelimumab, rituximab, ocrelizumab, obinutuzumab, ofatumumab, ibritumomab tiuxetan, tositumomab, ublituximab, and bevacizumab.

“Immunoglobulin” refers to a protein having the structure of a naturally occurring antibody. As an example, immunoglobulins of the IgG class are heterotetrameric glycoproteins with two light chains and two heavy chains that are disulfide-bonded. From N- to C-terminus, the heavy chains each have a variable region (VH), also called a variable heavy domain or a heavy chain variable domain, followed by three constant domains (CH1, CH2, and CH3), also called a heavy chain constant region. Similarly, from N- to C-terminus, light chain each have a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the sequence of its constant domain. An immunoglobulin includes two Fab molecules and an Fc domain, linked via the immunoglobulin hinge region.

“Fab molecule” or “antigen binding fragment” is an antigen binding fragment of an antibody that includes the variable domain and constant domain of a light chain, and a variable domain and a CH1 domain of a heavy chain.

“Single chain variable domain” or “scFv” refers to an antigen binding moiety that includes variable regions of a heavy chain and light chain, which are linked by a linker peptide.

“Bispecific antibody,” refers to an artificial antibody with two different antigen binding sites. Bispecific antibody can refer to a full immunoglobulin protein with two different antigen binding sites, or can refer to other molecules having two antigen binding moieties, such as a fusion protein including two Fabs or two scFvs.

“Diabody” refers to a class of antigen binding molecules that are bivalent and bispecific. The fragments comprise a heavy-chain variable domain (VH) connected to a light-chain variable domain (VL) on the same polypeptide chain (VH-VL). By using a linker that is too short to allow pairing between the two domains on the same chain, the domains are forced to pair with the complementary domains of another chain and create two antigen-binding sites.

“Bi-specific T-cell engager” refers to a class of bispecific antibodies having a first antigen binding moiety that binds to a T cell (e.g., by binding CD3), and a second antigen binding moiety that binds a different antigen (e.g., a tumor antigen).

“Nanobody” or “single domain antibody” refers to an antigen binding moiety that consists of a single monomeric variable antibody domain.

“Transferrin” is an iron transporter protein that may be used in a fusion protein to extend half-life. Human transferrin has a half-life of 12 days in serum.

“Cytokine” as used herein refers to a class of small (<25 kDa) proteins that are involved in cell signaling and immunomodulation. Cytokines include, for example, IL2, interleukin-10 (IL-10), interleukin-1 (IL-1), interleukin-17 (IL-17), interleukin-18 (IL-18), interferon α, interferon β, interferon γ, TGF-β1, TGF-β2, and TGF-β3, chemokine (C-C motif) ligand 2 (CCL2), and chemokine (C-C motif) ligand 19 (CCL19).

A “subject” according to any of the above embodiments is a mammal. Mammals include but are not limited to, domesticated animals (e.g., cows, sheep, cats, dogs, and horses), primates (e.g., human and non-human primates such as monkeys), rabbits, and rodents (e.g., mice and rats). Preferably the subject is a human.

“Modulating an immune response” may include one or more of a general increase, an increase in T effector cell response (e.g., cytotoxicity against tumor cells and virus infected cells), an increase in B cell activation, restoration of lymphocyte activation and proliferation, an increase in the expression of IL2 receptors, an increase in T cell responsiveness, an increase in natural killer cell activity or lymphokine-activated killer (LAK) cell activity, a decrease in regulatory T cells response to other T cells, and the like.

“Regulatory T cell” or “Treg cell” refers to a specialized type of CD4+ T cell that can function to suppress the responses of other T cells. Treg cells express the α-subunit of the IL2 receptor (CD25) and the transcription factor forkhead box P3 (FOXP3) (Sakaguchi, Annu Rev Immunol 22, 531-62 (2004)), and are involved in the induction and maintenance of peripheral self-tolerance to antigens, including those expressed by tumors. Treg cells require IL2 for their function and development and induction of their suppressive characteristics.

“T effector cells” refers to a population of T cells that respond to stimulus, such as IL2. T effector cells include CD8+ cytotoxic T cells and CD4+ helper T cells. As used herein, T effector cell does not include a T regulatory cell.

“Natural Killer cells” or “NK cells” are a component of the innate immune system and are cytotoxic lymphocytes that play a major role in rejection of tumors and virus infected cells.

“Treatment,” “treating” or “ameliorating” refers to medical management of a condition, disease, or disorder of a subject (e.g., patient), which may be therapeutic, prophylactic/preventative, or a combination treatment thereof.

An “effective amount” or a “therapeutically effective amount” may refer to an amount of therapeutic agent (e.g., an engineered IL2 polypeptide or engineered IL2 fusion polypeptide described herein) that provides a desired physiological change, such as an anti-cancer effect. The desired physiological change may, for example, be a decrease in symptoms of a disease, or a decrease in severity of a disease, or may be a reduction in the progression of a disease. With respect to cancer, the desired physiological changes may include, for example, tumor regression, a decreased rate of tumor progression, a reduced level of a cancer biomarker, reduced symptoms associated with cancer, a prevention or delay in metastasis, or clinical remission.

“Checkpoint inhibitor” refers to an agent that reduces the activity of an immune checkpoint protein. A checkpoint inhibitor can be an antigen binding moiety that binds to and reduces activity of an immune checkpoint protein. Immune checkpoint proteins include, for example, programmed cell death protein 1 (PD-1 or CD279), programmed death-ligand 1 (PD-L1 or CD274), cytotoxic T-lymphocyte-associated antigen 4 (CTLA-4 or CD152), T-cell immunoglobulin mucin-3 (TIM3), Lymphocyte Activating 3 (LAG3 or CD223), B7-H2 (ICOSL or CD275), and B7-H3 (CD276). Examples of checkpoint inhibitors includes ipilimumab (an anti-CTLA-4 antibody), nivolumab (an anti-PD-1 antibody), and pembrolizumab (an anti-PD-1 antibody).

“Cancer antigen” refers to a molecule that is preferentially expressed by cancer cells. Examples of cancer antigens include CD19, CD20, ROR1, fibroblast activation protein-α, and carcinoembryonic antigen (CEA).

“Oncolytic virus” refers to a virus that preferentially infects and kills cancer cells. For example oncolytic herpes viruses have been engineered to delete ICP34.5, resulting in a virus that only replicates in cancer (not healthy cells). An example of an oncolytic virus is Talimogene laherparepvec, which is used to treat melanoma.

“Cancer vaccine” refers to a vaccine that presents a cancer epitope to the immune system to elicit an anti-cancer response from the immune system. For example, sipuleucel-T is a vaccine for metastatic prostate cancer, which targets the immune response to the prostate cancer antigen prostatic acid phosphatase (PAP).

“Chimeric antigen receptors” or “CARs” are engineered antigen binding receptors that, when expressed in certain types of immune cells, activate the immune cell upon antigen binding. CARs typically include an extracellular domain comprising an antigen binding moiety (e.g., an scFv), a transmembrane domain, and an intracellular immune signaling domain (e.g., including signaling domains from CD3, 4-1BB, and/or CD28). CARs may be expressed, for example, by T cells or NK cells, and may include an antigen binding moiety that targets a cancer antigen, such as CD19 or ROR1.

“Tumor infiltrating lymphocyte” or “TIL” refers to a lymphocyte that is isolated from tumor tissue, manipulated in vitro (e.g., stimulated using a cytokine such as interleukin-2), and then infused back into a patient so that the activating TIL returns to the tumor site and induces tumor regression.

“Tumor microenvironment inhibitor” refers to an agent that inhibits one or more conditions or cell types that promote tumor growth and are present in the local environment surrounding a tumor. For example, bevacizumab can inhibit the tumor microenvironment by reducing angiogenesis in a tumor microenvironment.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

Recombinant DNA, molecular cloning, and gene expression techniques used in the present disclosure are known in the art and described in references, such as Sambrook et al., Molecular Cloning: A Laboratory Manual, 3^{rd} Ed., Cold Spring Harbor Laboratory, New York, 2001, and Ausubel et al., Current Protocols in Molecular Biology, John Wiley and Sons, Baltimore, Md., 1999.

### Engineered Interleukin-2 Polypeptide

As noted above, IL2 polypeptides of the present disclosure include IL2Rβ agonists having an engineered IL2 receptor β (IL2Rβ) binding region 2. In some embodiments, the binding region 2 comprises:

(SEQ ID NO: 1)
X₁-X₂-X₃-D-X₄-X₅-X₆-N-X₇-X₈-X₉-X₁₀-X₁₁-X₁₂-X₁₃,

wherein X₁, X₃, X₆, X₈, X₁₂, and X₁₃ each comprise any residue,

wherein X₂, X₄, and X₁₀ are uncharged residues, and

wherein X₅, X₇, X₉, and X₁₁ each comprise uncharged, nonpolar residues.

For example, in some embodiments the engineered IL2 polypeptide has the amino acid sequence:

(SEQ ID NO: 22)
APTSSSTKKTQLQLEHLLLDLQMILNGINNYKNPKLTRMLTFKFYMPKKA
TELKHLQCLEEELKPLEEVLNLAQSKNFHLX₁X₂X₃DX₄X₅X₆NX₇X₈X₉
X₁₀X₁₁X₁₂X₁₃LKGSETTFMCEYADETATIVEFLNRWITFCQSIISTL
T,

wherein X₁, X₃, X₆, X₈, X₁₂, and X₁₃ each comprise any residue,

wherein X₂, X₄, and X₁₀ are uncharged residues, and

wherein X₅, X₇, X₉, and X₁₁ each comprise uncharged, nonpolar residues.

In certain embodiments, X₁ is an uncharged polar residue, an uncharged nonpolar residue, a basic residue, or an acidic residue. In some embodiments, X₁ is selected from C, T, G, W, I, S, E, and K. In some embodiments, X₁ is selected from G, K, E, C, and T. In certain embodiments, X₂ is an uncharged polar residue or an uncharged nonpolar residue. In some embodiments, X₂ is selected from Y, P, V, W, L, A, and G. In some embodiments, X₂ is selected from V, P, W, and A. In certain embodiments, X₃ is an uncharged polar residue, an uncharged nonpolar residue, a basic residue, or an acidic residue. In some embodiments, X₃ is selected from S, T, Q, G, M, E, R, and K. In some embodiments, X₃ is selected from T, G, S, R, and E. In certain embodiments, X₄ is not L. In some embodiments, X₄ is an uncharged nonpolar residue or an uncharged polar residue. In some embodiments, X₄ is selected from A, V, S, and T. In certain embodiments, X₅ is selected from I, L, T, and V. In some embodiments, X₅ is selected from I and V. In certain embodiments, X₆ is an uncharged polar residue, a basic residue, or an acidic residue. In some embodiments, X₆ is selected from S, T, E, D, and R. In some embodiments, X₆ is selected from S, D, E, and T. In certain embodiments, X₇ is selected from I, A, M, and V. In some embodiments, X₇ is selected from I, A, and M. In certain embodiments, X₈ is an uncharged polar residue, an uncharged nonpolar residue, a basic residue, or an acidic residue. In some embodiments, X₈ is selected from S, T, N, Q, I, G, E, K, and R. In some embodiments, X₈ is selected from I, R, N, and T. In certain embodiments, X₉ is selected from V, L, and I. In some embodiments, X₉ is V. In certain embodiments, X₁₀ is an uncharged polar residue or an uncharged nonpolar residue. In some embodiments, X₁₀ is selected from N, T, I, and L. In some embodiments, X₁₀ is selected from I and L. In certain embodiments, X₁₁ is selected from V, A, and I. In certain embodiments, X₁₂ is an uncharged polar residue, an uncharged nonpolar residue, or an acidic residue. In some embodiments, X₁₂ is selected from Q, L, G, K, and R. In some embodiments, X₁₂ is selected from R, G, Q, and K. In certain embodiments, X₁₃ is an uncharged nonpolar residue or a basic residue. In some embodiments, X₁₃ is selected from A, D, and E. In some embodiments, X₁₃ is selected from E and A.

In some embodiments, the IL2Rβ binding region 2 is selected from:

(SEQ ID NO: 2)
GVTDSISNAIVLARE;
(SEQ ID NO: 3)
KWGDAVSNARVLAGE;
(SEQ ID NO: 4)
KWGDAVSNARVLAGA;
(SEQ ID NO: 5)
TLMDTTDNIGVLVRE;
(SEQ ID NO: 6)
EPSDVISNINVLVQE;
(SEQ ID NO: 7)
SPQDSIENISVLVRE;
(SEQ ID NO: 8)
WASDSIENITLLIQE;
(SEQ ID NO: 9)
CPTDTIENITVLIQE;
(SEQ ID NO: 10)
RYKDSLENMQIIIQE;
(SEQ ID NO: 11)
TARDAVDNMRVIIQE;
(SEQ ID NO: 12)
TPRDVVENMNVLVLE;
(SEQ ID NO: 13)
TPSDVIENMEVLILD;
(SEQ ID NO: 14)
TPSDAIENINVLIRE;
(SEQ ID NO: 15)
TPSDVIENITVLVQE;
(SEQ ID NO: 16)
GVGDTIDNINVLVKE;
(SEQ ID NO: 17)
IGRDSIDNIKVIVQE;
(SEQ ID NO: 18)
WATDTIRNVEVLVQE;
(SEQ ID NO: 19)
TAEDVVTNITVLVQE;
(SEQ ID NO: 20)
TAEDVISNIRVNVQE;
(SEQ ID NO: 21)
TPSDVIDNVSITVQE;
(SEQ ID NO: 210)
TARDAISNIRVIVQE;
(SEQ ID NO: 211)
RARDAIDNIRVIVQE;
(SEQ ID NO: 212)
TPRDAIDNINVIIQE;
(SEQ ID NO: 213)
TPRDAIDNIRVIVQE;
(SEQ ID NO: 214)
TPRDAIDNIRVIILE;
(SEQ ID NO: 215)
TARDAISNINVIIQE;
and
(SEQ ID NO: 216)
TARDAIDNINVIVQE;
and
(SEQ ID NO: 217)
TARDAIDNIRVIVLE.

In some embodiments, the engineered IL2Rβ binding region 2 is selected from: TPRDAIDNIRVIVQE (SEQ ID NO: 213); TPRDAIDNIRVIILE (SEQ ID NO:214); TARDAISNINVIIQE (SEQ ID NO: 215); and TARDAIDNINVIVQE (SEQ ID NO: 216).

In some embodiments, the engineered IL2Rβ binding region 2 is selected from: GVTDSISNAIVLARE (SEQ ID NO:2); KWGDAVSNARVLAGA (SEQ ID NO:4); EPSDVISNINVLVQE (SEQ ID NO:6); CPTDTIENITVLIQE (SEQ ID NO: 9); TARDAVDNMRVIIQE (SEQ ID NO:11); GVGDTIDNINVLVKE (SEQ ID NO: 16); TAEDVVTNITVLVQE (SEQ ID NO:19).

In some embodiments, the engineered IL2Rβ binding region 2 is selected from: GVTDSISNAIVLARE (SEQ ID NO:2); CPTDTIENITVLIQE (SEQ ID NO: 9); and TARDAVDNMRVIIQE (SEQ ID NO:11).

In some embodiments, the engineered IL2 polypeptide has an amino acid sequence of SEQ ID NO: 22. In some embodiments, the engineered IL2 polypeptide has an amino acid sequence of any one of SEQ ID NOs: 23-42.

In some embodiments, the IL2Rβ agonists of the present disclosure include engineered IL2 polypeptides comprising a substitution to at least one residue selected from: R81, P82, R83, L85, I86, S87, I89, N90, I92, V93, and L94, relative to SEQ ID NO: 65. In certain embodiments, the at least one substitution is a substitution to residue L85. In certain embodiments, the IL2 polypeptide includes substitutions to at least two residues selected from: R81, P82, R83, L85, I86, S87, I89, N90, I92, V93, and L94. In some embodiments, the at least two residues are selected from R81, R83, L85, I92, and L94. In some embodiments, the IL2 polypeptide includes substitutions to at least three residues selected from R81, R83, L85, I92, and L94. In some embodiments, the engineered IL2 polypeptide includes substitutions to R81, R83, L85, I92, and L94. In some embodiments, the R81 substitution is selected from R81G, R81K, R81E, R81C, and R81T. In some embodiments, the R83 substitution is selected from R83T, R83G, R83S, and R83E. In some embodiments, the L85 substitution is selected from L85S, L85A, L85V, and L85T. In some embodiments, the I92 substitution is I92L. In some embodiments, the L94 substitution is selected from L94R, L94G, L94Q, and L94K. In some embodiments, the engineered IL2 polypeptide comprising a substitution to at least one residue selected from: R81, P82, R83, L85, I86, S87, I89, N90, I92, V93, and L94 has an IL2Rβ binding region 2 of any of SEQ ID NOs: 2-24.

In some embodiments, the engineered IL2 polypeptide has increased affinity for IL2Rβ as compared to the wild-type IL2. In certain embodiments, the binding of the engineered IL2 polypeptide to IL2Rβ has a K_{D} at least 10-fold greater, at least 15-fold greater, at least 20-fold greater, at least 25-fold greater, or at least 30-fold greater than binding of a wild-type IL2 to IL2Rβ. In some embodiments, the engineered IL2 polypeptide binds to IL2Rβ with a K_{D} at least 30-fold greater than a wild-type IL2. In some embodiments, the engineered IL2 polypeptide has at least a 10-fold increase, at least a 15-fold increase, at least a 20-fold increase, at least a 25-fold increase, or at least a 30-fold increase in affinity for IL2Rβ as compared to wild-type IL2.

In some embodiments, the engineered IL2 polypeptide has a decrease in affinity for IL2Rα as compared to wild-type IL2. In certain embodiments, the engineered IL2 polypeptide has at least a 5% decrease, at least a 10% decrease, at least a 15% decrease, or at least a 20% decrease in affinity for IL2Rα as compared to wild-type IL2.

In some embodiments, the engineered IL2 polypeptide has a similar affinity for IL2Rα as compared to wild-type IL2. In certain embodiments, the engineered IL2 polypeptide has an affinity for IL2Rα that varies from the affinity of wild-type IL2 for IL2Rα by no more than ±20%, no more than ±15%, no more than ±10% or no more than ±5%.

Some embodiments of the present disclosure provide an engineered IL2 polypeptide comprising an engineered IL2 receptor α (IL2Rα) binding region 1. The engineered IL2Rα binding region 1 can comprise a substitution selected from: a substitution at position K35, a substitution at R38, a substitution at F42, a substitution at Y45, or combinations thereof. In some embodiments, the engineered IL2 polypeptide binds to IL2Rα with at least 2-fold reduced binding kinetics as compared to wild-type IL2.

In some embodiments, the engineered IL2 polypeptide may comprise a substitution at position K35. In some embodiments, the substitution at position K35 comprises a non-basic residue. In some embodiments, the substitution at position K35 comprises an uncharged residue or an acidic residue. In some embodiments, the substitution at position K35 is selected from: K35G, K35L, K35S, K35V, K35D, K35E, and K35C.

In some embodiments, the engineered IL2 polypeptide comprises a substitution at position R38. In some embodiments, the substitution at position R38 comprises a non-basic residue. In some embodiments, the substitution at position R38 comprises an uncharged residue or an acidic residue. In some embodiments, the substitution at position R38 is selected from: R38V, R38D, R38E, R38S, R38I, R38A, R38Y, R38G, R38C, and R38N.

In some embodiments, the engineered IL2 polypeptide may comprise a substitution at position F42. In some embodiments, the substitution at position F42 comprises an uncharged residue. In some embodiments, the substitution at position F42 comprises a basic residue. In some embodiments, the substitution at position F42 is selected from: F42A, F42R, F42G, F42I, F42L, F42P and F42H.

In some embodiments, the engineered IL2 polypeptide may comprise a substitution at position Y45. In some embodiments, the substitution at position Y45 comprises an uncharged residue. In some embodiments, the substitution at position Y45 comprises an uncharged polar residue or an uncharged non-polar residue. In some embodiments, the Y45 substitution is Y45S, Y45P, Y45A, Y45V, Y45C, Y45T, and Y45F.

In some embodiments, the engineered IL2 polypeptide may comprise a substitution at position K35 and a substitution at position R38. In some embodiments, the engineered IL2 polypeptide comprises a K35G substitution and R38E substitution.

In some embodiments, the engineered IL2 polypeptide may comprise a substitution at position K35 and a substitution at position F42. In some embodiments, the engineered IL2 polypeptide comprises a K35S substitution and an F42G substitution.

In some embodiments, the engineered IL2 polypeptide may comprise a substitution at position K35, a substitution at position R38, and a substitution at position F42. In some embodiments, the engineered IL2 polypeptide comprises a K35L substitution, an R38D substitution, and an F42R substitution.

In some embodiments, the engineered IL2 polypeptide may comprise a substitution at position R38 and a substitution at position Y45S. In some embodiments, the engineered IL2 polypeptide comprises an R38D substitution and an Y45S substitution. In some embodiments, the engineered IL2 polypeptide comprises an R38V substitution and an Y45S substitution.

In some embodiments, the engineered IL2 polypeptide binds to IL2Rα with at least 10-fold reduced binding kinetics as compared to wild-type IL2.

In some embodiments, the IL2Rα binding region 1 is selected from:

(SEQ ID NO: 183)
PVLTRMLTIKFY;
(SEQ ID NO: 184)
PKLTRMLTLKFP;
(SEQ ID NO: 185)
PDLTSMLAFKFY;
(SEQ ID NO: 186)
PGLTEMLTFKFY;
(SEQ ID NO: 187)
PSLTRMLTGKFY;
(SEQ ID NO: 188)
PELTIMLTPKFY;
(SEQ ID NO: 189)
PCLTAMLTLKFA;
(SEQ ID NO: 190)
PCLTAMLTLKFA;
(SEQ ID NO: 191)
PKLTRMLTHKFV;
(SEQ ID NO: 192)
PCLTDMLTFKFY;
(SEQ ID NO: 193)
PLLTDMLTRKFY;
(SEQ ID NO: 194)
PLLTDMLTFKFY;
(SEQ ID NO: 195)
PKLTDMLTFKFS;
(SEQ ID NO: 196)
PKLTYMLTRKFY;
(SEQ ID NO: 197)
PKLTRMLTFKFC;
(SEQ ID NO: 198)
PKLTSMLTFKFS;
(SEQ ID NO: 199)
PKLTSMLTFKFS;
(SEQ ID NO: 200)
PKLTYMLTFKFS;
(SEQ ID NO: 201)
PKLTYMLTFKFS;
(SEQ ID NO: 202)
PKLTGMLTFKFS;
(SEQ ID NO: 203)
PKLTVMLTFKFT;
(SEQ ID NO: 204)
PKLTYMLTFKFS;
(SEQ ID NO: 205)
PKLTVMLTFKFP;
(SEQ ID NO: 206)
PKLTVMLTFKFF;
(SEQ ID NO: 207)
PKLTCMLTFKFA;
(SEQ ID NO: 208)
PKLTNMLTFKFA;
and
(SEQ ID NO: 209)
PKLTNMLTFKFS.

In some embodiments, the engineered IL2 polypeptide shares at least 80%, for example, at least 85%, at least 88%, at least 90%, at least 92%, at least 95%, at least 96%, at least 97%, at least 98%, at least 99% or at least 100% sequence identity with the residues outside of the IL2Rβ binding region 2 (i.e., residues 1-80 and 96-133) of SEQ ID NO: 22 and binds to IL2Rβ. In some embodiments, the engineered IL2 polypeptide shares at least 80%, for example, at least 85%, at least 88%, at least 90%, at least 92%, at least 95%, at least 96%, at least 97%, at least 98%, at least 99% or at least 100% sequence identity with the residues outside of the IL2Rα binding region 1 (i.e., residues 1-33 and 46-133) of SEQ ID NO: 223 and has reduced binding to IL2Rα and binds to IL2Rβ. In some embodiments, the engineered IL2 polypeptide shares at least 80%, for example, at least 85%, at least 88%, at least 90%, at least 92%, at least 95%, at least 96%, at least 97%, at least 98%, at least 99% or at least 100% sequence identity with the residues outside of the IL2Rα binding region 1 and IL2Rβ binding region 2 (i.e., residues 1-33, 46-80, 96-133) of SEQ ID NO: 22 or SEQ ID NO: 223 and has reduced binding to IL2Rα and binds to IL2Rβ. In some embodiments, the present disclosure provides an engineered IL2 polypeptide including an engineered IL2Rβ binding region 2 as previously described and an engineered IL2Rα binding region 1 as previously described. In certain embodiments, the engineered IL2 polypeptide comprises an engineered IL2Rα binding region 1 selected from PVLTRMLTIKFY (SEQ ID NO: 183); PKLTRMLTLKFP (SEQ ID NO:184); PDLTSMLAFKFY (SEQ ID NO:185); PGLTEMLTFKFY (SEQ ID NO:186); PSLTRMLTGKFY (SEQ ID NO:187); PELTIMLTPKFY (SEQ ID NO:188); PCLTAMLTLKFA (SEQ ID NO:189); PCLTAMLTLKFA (SEQ ID NO:190); PKLTRMLTHKFV (SEQ ID NO:191); PCLTDMLTFKFY (SEQ ID NO:192); PLLTDMLTRKFY (SEQ ID NO:193); PLLTDMLTFKFY (SEQ ID NO:194); PKLTDMLTFKFS (SEQ ID NO:195); PKLTYMLTRKFY (SEQ ID NO:196); PKLTRMLTFKFC (SEQ ID NO:197); PKLTSMLTFKFS (SEQ ID NO:198); PKLTSMLTFKFS (SEQ ID NO:199); PKLTYMLTFKFS (SEQ ID NO:200); PKLTYMLTFKFS (SEQ ID NO:201); PKLTGMLTFKFS (SEQ ID NO:202); PKLTVMLTFKFT (SEQ ID NO:203); PKLTVMLTFKFS (SEQ ID NO:204); PKLTVMLTFKFP (SEQ ID NO:205); PKLTVMLTFKFF (SEQ ID NO:206); PKLTCMLTFKFA (SEQ ID NO:207); PKLTNMLTFKFA (SEQ ID NO:208); and PKLTNMLTFKFS (SEQ ID NO:209); and comprises an engineered IL2Rβ binding region 2 is selected from:

(SEQ ID NO: 2)
GVTDSISNAIVLARE;
(SEQ ID NO: 3)
KWGDAVSNARVLAGE;
(SEQ ID NO: 4)
KWGDAVSNARVLAGA;
(SEQ ID NO: 5)
TLMDTTDNIGVLVRE;
(SEQ ID NO: 6)
EPSDVISNINVLVQE;
(SEQ ID NO: 7)
SPQDSIENISVLVRE;
(SEQ ID NO: 8)
WASDSIENITLLIQE;
(SEQ ID NO: 9)
CPTDTIENITVLIQE;
(SEQ ID NO: 10)
RYKDSLENMQIIIQE;
(SEQ ID NO: 11)
TARDAVDNMRVIIQE;
(SEQ ID NO: 12)
TPRDVVENMNVLVLE;
(SEQ ID NO: 13)
TPSDVIENMEVLILD;
(SEQ ID NO: 14)
TPSDAIENINVLIRE;
(SEQ ID NO: 15)
TPSDVIENITVLVQE;
(SEQ ID NO: 16)
GVGDTIDNINVLVKE;
(SEQ ID NO: 17)
IGRDSIDNIKVIVQE;
(SEQ ID NO: 18)
WATDTIRNVEVLVQE;
(SEQ ID NO: 19)
TAEDVVTNITVLVQE;
(SEQ ID NO: 20)
TAEDVISNIRVNVQE;
(SEQ ID NO: 21)
TPSDVIDNVSITVQE;
(SEQ ID NO: 210)
TARDAISNIRVIVQE;
(SEQ ID NO: 211)
RARDAIDNIRVIVQE;
(SEQ ID NO: 212)
TPRDAIDNINVIIQE;
(SEQ ID NO: 213)
TPRDAIDNIRVIVQE;
(SEQ ID NO: 214)
TPRDAIDNIRVIILE;
(SEQ ID NO: 215)
TARDAISNINVIIQE;
and
(SEQ ID NO: 216)
TARDAIDNINVIVQE;
and
(SEQ ID NO: 217)
TARDAIDNIRVIVLE.

In particular embodiments, the engineered IL2 polypeptide includes an engineered IL2Rβ binding region 2 selected from GVTDSISNAIVLARE (SEQ ID NO: 2); TARDAVDNMRVIIQE (SEQ ID NO: 11); TPRDAIDNIRVIVQE (SEQ ID NO: 213); TPRDAIDNIRVIILE (SEQ ID NO:214); TARDAISNINVIIQE (SEQ ID NO: 215); and TARDAIDNINVIVQE (SEQ ID NO: 216); and an engineered IL2Rα binding region 1 as previously described.

In some embodiments, the engineered IL2 polypeptide is selected from any one of SEQ ID NOs: 147-170, with the C-terminal histidine tag optionally included (or excluded). In some embodiments, the C-terminal histidine tag is replaced another linker, such as a gly-ser linker.

### Engineered IL2 Fusion Polypeptides

Some embodiments of the present disclosure provide engineered IL2 fusion polypeptides. The engineered IL2 fusion polypeptide may include an engineered IL2 polypeptide as previously described herein, and at least one additional molecule covalently attached to the engineered IL2 polypeptide via a peptide bond or other chemical linkage. In some embodiments, the at least one additional molecule of the fusion polypeptide is a half-life extending molecule. In some embodiments, the half-life extending molecule comprises a half-life extending polypeptide. In some embodiments, the half-life extending polypeptide comprises an Fc domain, human serum albumin (HSA), an HSA binding molecule, or transferrin.

In certain embodiments, the IL2 fusion polypeptide comprises an Fc domain. In some embodiments, the Fc domain is derived from an IgG antibody. Human IgG antibodies have several subclasses, including, but not limited to IgG1, IgG2, IgG3, and IgG4. In particular embodiments, the Fc domain is derived from an IgG1 antibody or an IgG4 antibody. In some embodiments, the Fc domain has one or more substitutions that reduce effector function of the Fc domain. Examples of substitutions that reduce Fc effector function include L34A, L235A, and P329G. “LALAPG” may refer to a modified Fc domain including each of L34A, L235A, and P329G. In some embodiments, the Fc domain comprises at least one amino acid residue modification to increase serum half-life. Representative modifications to the Fc domain are described in U.S. Pat. Nos. 7,317,091; 8,735,545; 7,371,826; 7,670,600; and 9,803,023. In some embodiments, the Fc domain is SEQ ID NO: 64. In some embodiments, the engineered IL2-Fc fusion polypeptide comprises a sequence selected from SEQ ID NOs: 39-49.

In some embodiments, the at least one additional molecule of the fusion polypeptide is an antigen binding moiety. In some embodiments, the antigen binding moiety comprises an immunoglobulin, a Fab molecule, an scFv, a bi-specific T-cell engager, a diabody, a single domain antibody, or a nanobody. An antigen binding moiety may bind, for example, carcinoembryonic antigen (CEA), GD2, or CD20. An example of a CEA antigen moiety is CH1A1A-2F. An example of a GD-2 antigen binding moiety is dinutuximab, and an example of a CD20 antigen binding moiety is rituximab.

In some embodiments, the at least one additional molecule of the fusion polypeptide is a cytokine. In some embodiments, the cytokine is selected from interleukin-2, interleukin-15, interleukin-7, interleukin-10, and C-C motif chemokine ligand 19 (CCL19). In some embodiments, the additional molecule of the fusion polypeptide is a second engineered IL2 polypeptide as described herein.

In some embodiments, the half-life extending molecule comprises poly-ethylene glycol (PEG) or polypropylene glycol (PPG).

In some embodiments, the fusion polypeptide is a monovalent fusion polypeptide. A monovalent fusion polypeptide refers to a fusion polypeptide that has one copy of an engineered IL2 polypeptide.

In certain embodiments, the monovalent fusion polypeptide includes an engineered IL2 polypeptide linked to a fusion partner, such as an Fc region. A variety of linkers are known in the art and may be used to covalently link an engineered IL2 described herein to a fusion partner, such as an Fc region. By “linker”, “linker sequence”, herein is meant a molecule or group of molecules (such as a monomer or polymer) that connects two molecules and often serves to place the two molecules in a preferred configuration. The linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. Useful linkers include glycine-serine polymers (including, for example, (GS)n, (GSGGS)n (SEQ ID NO:218), (GGGGS)n (SEQ ID NO:219) and (GGGS)n (SEQ ID NO:220), where n is an integer of at least one), glycine-alanine polymers, alanine-serine polymers, and other flexible linkers. In some embodiments, the fusion polypeptide is a bivalent fusion polypeptide. A bivalent fusion protein may refer to a molecular complex that includes two copies of engineered IL2 polypeptides, which may be of the same sequence or different sequences. The molecular complex may be bound non-covalently. For example, a bivalent fusion protein may include two Fc regions bound together non-covalently such as by one or more disulfide bridges, or by knobs-into-holes chemistry.

### Methods of Making Engineered IL2 Polypeptides

Engineered IL2 polypeptides or engineered IL2 fusion polypeptides can be prepared by genetic or chemical methods well known in the art and by the methods disclosed in the Examples below. Genetic methods may include, for example, site-specific mutagenesis of the DNA sequence encoding the polypeptide, PCR, and gene synthesis. The intended nucleotide changes can be verified by sequencing. The nucleotide sequence of native IL2 has been described by Taniguchi et al. (Nature 302, 305-10 (1983)) and a nucleic acid encoding native human IL2 is available from, for example, American Type Culture Collection (Rockville Md.).

Engineered IL2 polypeptides or engineered IL2 fusion polypeptides may be obtained, for example, by recombinant production or solid-state peptide synthesis. For recombinant production, a polynucleotide encoding an engineered IL2 polypeptide or engineered IL2 fusion polypeptide can be isolated and inserted into one or more vectors for cloning and/or expression in a host cell. Such polynucleotides may be readily isolated and sequenced by conventional procedures. In certain embodiments, a vector, such as an expression vector, comprising one or more of the polynucleotides of the instant disclosure is provided. Methods which are well known to those skilled in the art can be used to construct expression vectors containing the coding sequence engineered IL2 polypeptide or engineered IL2 fusion polypeptide along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL (FOURTH EDITION), Cold Spring Harbor Laboratory, N.Y. (2012); and Ausubel et al., CURRENT PROTOCOLS IN MOLECULAR BIOLOGY, Greene Publishing Associates and Wiley Interscience, N.Y (1993). The expression vector can be part of a plasmid, virus, or may be a nucleic acid fragment. The expression vector includes an expression cassette into which the polynucleotide encoding engineered IL2 polypeptide or engineered IL2 fusion polypeptide (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the disclosed herein may encode one or more polyproteins, which are post- or co-translationally separated into the final proteins via proteolytic cleavage. In addition, a vector, polynucleotide, or nucleic acid of the instant disclosure may encode heterologous coding regions, either fused of unfused to a first or second polynucleotide encoding the polypeptides disclosed herein, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (e.g., Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present disclosure may be associated with additional coding regions that encode secretory or signal peptides, which direct the secretion of a polypeptide encoded by a polynucleotide of the present disclosure. For example, if secretion of the engineered IL2 polypeptide or engineered IL2 fusion polypeptide is desired, DNA encoding a signal sequence may be placed upstream of the nucleic acid encoding the mature amino acids of the an engineered IL2 polypeptide or engineered IL2 fusion polypeptide. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. For example, native human IL2 is translated with a 20 amino acid signal sequence at the N-terminus of the polypeptide, which is subsequently cleaved off to produce mature, 133 amino acid human IL2. In some embodiments, the native signal peptide, e.g. the IL2 signal peptide or an immunoglobulin heavy chain or light chain signal peptide is used, or a functional derivative of that sequence that retains the ability to direct the secretion of the polypeptide that is operably associated with it.

In some embodiments, a polynucleotide encoding the engineered IL2 polypeptide or engineered IL2 fusion polypeptide further includes a DNA sequence encoding a sequence to facilitate purification (e.g., a histidine tag) or for labeling the engineered IL2 polypeptide or engineered IL2 fusion polypeptide within or at the ends of the polynucleotide encoding the engineered IL2 polypeptide or engineered IL2 fusion polypeptide.

In certain embodiments, a host cell comprising one or more polynucleotides encoding an engineered IL2 polypeptide or engineered IL2 fusion polypeptide is provided. In certain embodiments, the host cell comprises one or more vectors encoding the engineered IL2 polypeptide or engineered IL2 fusion. The host cell can be any kind of cellular system that can be used to generate the engineered IL2 polypeptide or engineered IL2 fusion polypeptide. Such cells may be transfected or transduced as appropriate with the particular expression vector encoding the engineered IL2 polypeptide or engineered IL2 fusion, and large quantities of vector containing cells can be grown for seeding large scale fermenters to obtain sufficient quantities of encoding the engineered IL2 polypeptide or engineered IL2 fusion for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or lymphoid cell (e.g., Y0, NS0, Sp20 cell).

Standard technologies are known in the art to express foreign genes in these systems. Cells expressing an engineered IL2 polypeptide fused to either the heavy or the light chain of an antigen binding moiety, such as an antibody, may be engineered so as to also express the other of the antibody chains such that the expressed engineered IL2 fusion polypeptide comprises an antibody that has both a heavy and a light chain.

In some embodiments, a method of producing an engineered IL2 polypeptide or engineered IL2 fusion polypeptide is provided. In some embodiments, the method comprises culturing a host cell comprising a polynucleotide encoding the an engineered IL2 polypeptide or engineered IL2 fusion polypeptide, as provided herein, under conditions suitable for expression of the an engineered IL2 polypeptide or engineered IL2 fusion polypeptide, and optionally recovering and/or purifying the an engineered IL2 polypeptide or engineered IL2 fusion polypeptide from the host cell (or host cell culture medium, for example, if the host cell secretes the polypeptide).

### Pharmaceutical Compositions

Provided herein are pharmaceutical compositions comprising an engineered IL2 polypeptide or engineered IL2 fusion polypeptide as described herein and a pharmaceutically acceptable diluent(s), excipient(s), or carrier(s). In some embodiments, the pharmaceutical compositions comprise an engineered IL2 polypeptide or an engineered IL2 fusion polypeptide as disclosed herein and an additional therapeutic agent (e.g., combination therapy). Non-limiting examples of such therapeutic agents are described herein below. The pharmaceutical compositions may be formulated in a conventional manner using one or more pharmaceutically acceptable carriers comprising excipients and auxiliaries which facilitate processing of the engineered IL2 or IL2 fusion polypeptide into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Examples of IL-2 compositions are described in U.S. Pat. Nos. 4,604,377 and 4,766,106, which are incorporated by reference herein.

As used herein, “pharmaceutically acceptable carrier” and “physiologically acceptable carriers” are used interchangeably and include any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as, for example, a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. Engineered IL2 polypeptides or engineered IL2 fusion polypeptides as describe herein (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the engineered IL2 polypeptides or engineered IL2 fusion polypeptides describe herein.

Parenteral compositions include those designed for administration by injection, e.g. subcutaneous, intradermal, intralesional, intravenous, intraarterial intramuscular, intrathecal or intraperitoneal injection. For injection, the engineered IL2 polypeptides or engineered IL2 fusion polypeptides described herein may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the engineered IL2 polypeptides or engineered IL2 fusion polypeptides may be in powder form for constitution with a suitable vehicle, e.g., sterile pyrogen-free water, before use. Sterile injectable solutions are prepared by incorporating the engineered IL2 polypeptides or engineered IL2 fusion polypeptides in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, methods of preparation include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The pharmaceutical composition is preferably stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. In some embodiments, the suspension may also contain suitable stabilizers or agents, which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

In some embodiments, aqueous suspensions contain one or more polymers as suspending agents. Example polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the pharmaceutical compositions include solubilizing agents to aid in the solubility of the engineered IL2 polypeptide or engineered IL2 fusion polypeptide. The term “solubilizing agent” generally includes agents that result in formation of a micellar solution or a true solution of the agent. Certain acceptable nonionic surfactants, for example polysorbate 80, are useful as solubilizing agents. Examples include glycols, polyglycols, e.g., polyethylene glycol 400, and glycol ethers.

In some embodiments, the pharmaceutical compositions include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the pharmaceutical compositions include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the pharmaceutical compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In some embodiments, the engineered IL2 polypeptides or engineered IL2 fusion polypeptides described herein are delivered using a sustained-release system, such as semipermeable matrices of solid hydrophobic polymers containing the therapeutic agent. Various sustained-release materials are useful herein. In some embodiments, sustained-release capsules release the engineered IL2 polypeptide or engineered IL2 fusion polypeptides for a few weeks up to over 100 days. Depending on the chemical nature and the biological stability of the therapeutic reagent, additional strategies for protein stabilization are employed. Examples of sustained-release preparations include semipermeable matrices of solid hydrophobic polymers containing the polypeptide, which matrices are in the form of shaped articles, e.g. films, or microcapsules. In some embodiments, prolonged absorption of an injectable composition can be brought about by the use in the compositions of agents delaying absorption, such as, for example, aluminum monostearate, gelatin or combinations thereof.

In some embodiments, engineered IL2 polypeptides or engineered IL2 fusion polypeptides described herein may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990).

In some embodiments, engineered IL2 polypeptides or engineered IL2 fusion polypeptides described herein may also be formulated as a depot preparation. Such long acting formulations may be administered by implantation (for example subcutaneously or intramuscularly) or by intramuscular injection. Thus, for example, the engineered IL2 polypeptides or engineered IL2 fusion polypeptides may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the engineered IL2 polypeptides or engineered IL2 fusion polypeptides described herein may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

In some embodiments, the engineered IL2 polypeptides or engineered IL2 fusion polypeptides may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

In some embodiments, the engineered IL2 polypeptides or engineered IL2 fusion polypeptides described herein are formulated for oral administration. In various embodiments, the engineered IL2 polypeptides or engineered IL2 fusion polypeptides described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the engineered IL2 polypeptides or engineered IL2 fusion polypeptides described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In some embodiments, dosage forms, such as dragee cores and tablets, are provided with one or more suitable coating. In specific embodiments, concentrated sugar solutions are used for coating the dosage form. The sugar solution, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active agent doses.

In certain embodiments, therapeutically effective amounts of at least one of the engineered IL2 polypeptides or engineered IL2 fusion polypeptides described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include lactose, binders such as starches, and/or lubricants such as talc or magnesium stearate and, optionally, stabilizers. In some embodiments, soft capsules contain one or more active agent that is dissolved or suspended in a suitable liquid. Suitable liquids may include one or more fatty oil, liquid paraffin, or liquid polyethylene glycol. In addition, stabilizers are optionally added.

In some embodiments, therapeutically effective amounts of at least one of the engineered IL2 polypeptides or engineered IL2 fusion polypeptides described herein are formulated for buccal or sublingual administration. Formulations suitable for buccal or sublingual administration include, by way of example only, tablets, lozenges, or gels.

In some embodiments, the engineered IL2 polypeptide or engineered IL2 fusion polypeptide is administered topically. The engineered IL2 polypeptide or engineered IL2 fusion polypeptide described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In some embodiments, the engineered IL2 polypeptides or engineered IL2 fusion polypeptides are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the engineered IL2 polypeptide or engineered IL2 fusion polypeptide is accomplished by means of iontophoretic patches and the like. In certain embodiments, transdermal patches provide controlled delivery of the engineered IL2 polypeptide or engineered IL2 fusion polypeptide. In specific embodiments, the rate of absorption is slowed by using rate-controlling membranes or by trapping the engineered IL2 polypeptide or engineered IL2 fusion polypeptide within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the engineered IL2 polypeptide or engineered IL2 fusion polypeptide optionally with carriers, optionally a rate controlling barrier to deliver the engineered IL2 polypeptide or engineered IL2 fusion polypeptide to the skin of the host at a controlled and predetermined rate over a prolonged period of time, and means to secure the device to the skin.

In some embodiments, the engineered IL2 polypeptides or engineered IL2 fusion polypeptides are formulated for administration by inhalation. Various forms suitable for administration by inhalation include, but are not limited to, aerosols, mists or powders. Pharmaceutical compositions of the engineered IL2 polypeptides or engineered IL2 fusion polypeptides may be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the engineered IL2 polypeptide or engineered IL2 fusion polypeptide and a suitable powder base such as lactose or starch.

In some embodiments, the engineered IL2 polypeptide or engineered IL2 fusion polypeptides are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of the engineered IL2 polypeptide or engineered IL2 fusion polypeptide provided in the pharmaceutical compositions of the present disclosure is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the engineered IL2 polypeptide or engineered IL2 fusion polypeptide provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the engineered IL2 polypeptide or engineered IL2 fusion polypeptide provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the engineered IL2 polypeptide or engineered IL2 fusion polypeptide provided in the pharmaceutical compositions of the present disclosure is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount the engineered IL2 polypeptide or engineered IL2 fusion polypeptide provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the engineered IL2 polypeptide or engineered IL2 fusion polypeptide provided in the pharmaceutical compositions of the present disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the engineered IL2 polypeptide or engineered IL2 fusion polypeptide provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

### Methods of Treatment and Use

In some embodiments of the present disclosure provided herein are methods of modulating an immune response in a subject in need thereof, comprising administering to the subject a therapeutically effective amount of an engineered IL2 polypeptide, an engineered IL2 fusion polypeptide, or a pharmaceutical composition thereof as previously described herein. In certain embodiments, modulating the immune response includes at least one of: enhancing effector T cell activity, enhancing NK cell activity, and suppressing regulatory T cell activity. In some embodiments of the present disclosure provided herein engineered IL2 polypeptides as previously described, fusion polypeptides as previously described, and/or pharmaceutical compositions as previously described, for use in a method of modulating an immune response in a subject in need thereof. In some embodiments, modulating the immune response comprising increasing STAT5 phosphorylation compared to WT IL2.

In some embodiments of the present disclosure is a method of treating a disease or condition in a subject in need thereof, comprising administering to the subject a therapeutically effective amount of an engineered IL2 polypeptide, an engineered IL2 fusion polypeptide, or a pharmaceutical composition thereof as previously described herein. In some embodiments of the present disclosure provided herein engineered IL2 polypeptides as previously described, fusion polypeptides as previously described, and/or pharmaceutical compositions as previously described, for use in a method of treating a subject for a disease. Non-limiting examples of diseases or condition contemplated in the method include proliferative disorders, such as cancer, and immunosuppression.

In some embodiments, is a method of treating a proliferative disorder comprising administering to the subject a therapeutically effective amount of an engineered IL2 polypeptide, an engineered IL2 fusion polypeptide, or a pharmaceutical composition thereof as previously described herein. In some embodiments, the proliferative disorder is cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, glioblastoma, prostate cancer, blood cancer, skin cancer, squamous cell carcinoma, skin cancer, melanoma, bone cancer, renal cell carcinoma, and kidney cancer. Also included are pre-cancerous conditions or lesions and cancer metastases. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Similarly, other cell proliferation disorders can also be treated, such as hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

In some embodiments, the method of treatment or modulating the immune response further comprises administering to the subject a therapeutically effective amount of at least one additional therapeutic agent (e.g., a combination therapy). In certain embodiments, the additional therapeutic agent is an anti-cancer agent. Examples of anti-cancer agents include checkpoint inhibitors (e.g., anti-PD1 antibodies), chemotherapeutic agents, agents that inhibit a tumor microenvironment, cancer vaccines (e.g., Sipuleucel-T), oncolytic viruses (e.g., talimogene laherparepvec), immune cells expressing a chimeric antigen receptor, and tumor infiltrating lymphocytes. In certain embodiments, the additional therapeutic agent is a molecule including an antigen binding moiety. In certain specific embodiments, the antigen binding moiety is selected from a single domain antibody, a Fab molecule, an scFv, a diabody, a nanobody, a bi-specific T cell engager, or an immunoglobulin. In certain embodiments, the antigen binding moiety is specific to a tumor antigen (e.g., carcinoembryonic antigen, fibroblast activation protein-α, CD20) or a check point protein (e.g., CTLA-4, PD-1 or PD-L1). In some embodiments, the additional therapeutic agent comprises an immune cell expressing a chimeric antigen receptor, an immune cell expressing an engineered T cell receptor, or a tumor infiltrating lymphocyte. In certain embodiments, the engineered IL2 polypeptide or engineered IL2 fusion polypeptide may be encoded by a polynucleotide transfected, transvected, or otherwise introduced into the immune cell that expresses the chimeric antigen receptor, the immune cell expressing an engineered T cell receptor, or the tumor infiltrating lymphocyte. In such embodiments, the immune cell may be an armored chimeric antigen receptor-expressing cell. The polynucleotide may additionally encode a secretion signal (e.g., the native IL2 signal sequence or a signal sequence derived from another protein) directly upstream of the engineered IL2 polypeptide coding sequence, to allow the cell to secrete the engineered IL2 polypeptide or engineered IL2 fusion polypeptide.

In some embodiments, the methods of treatment or modulating the immune response include administering to the subject an engineered IL2 polypeptide having an IL2Rβ binding region two of SEQ ID NO: 1. In certain embodiments, the methods of treatment or modulating the immune response include administering to the subject an engineered IL2 polypeptide having an IL2Rβ binding region two of any one of SEQ ID NOs: 2-21. In certain embodiments, the method includes administering to the subject an engineered IL2 polypeptide of any of SEQ ID NOs: 23-42, any one of SEQ ID NOs: 44-63, any one of SEQ ID NOs: 147-170 (with the C-terminal histidine tag optionally included), or an Fc fusion polypeptide of SEQ ID NO: 110, SEQ ID NO: 111, SEQ ID NO: 144 or SEQ ID NO: 145. In certain embodiments, the methods of treatment or modulating the immune response include administering to the subject an engineered IL2 polypeptide of SEQ ID NO: 22, or of any one of SEQ ID NOs: 23-42. In certain embodiments, the methods of treatment or modulating the immune response include administering to the subject an engineered IL2 fusion polypeptide of SEQ ID NO: 51. In certain embodiments, the methods of treatment or modulating the immune response include administering to the subject an engineered IL2 fusion polypeptide of SEQ ID NO: 43, or of any one of SEQ ID NOs: 44-63.

Suitable routes of administration include, but are not limited to, intravenous, parenteral, transdermal, oral, rectal, aerosol, ophthalmic, pulmonary, transmucosal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, an engineered IL2 polypeptide or IL2 fusion polypeptide is administered systemically. In certain embodiments, an engineered IL2 polypeptide or engineered IL2 fusion polypeptide as described herein is administered in a local rather than systemic manner, for example, via injection of the engineered IL2 polypeptide or engineered IL2 fusion polypeptide directly into an organ, tissue, or tumor. In some embodiments, long acting formulations are administered by implantation (for example subcutaneously or intramuscularly) or by intramuscular injection. Furthermore, in some embodiments, the drug is delivered in a targeted drug delivery system, for example, in a liposome coated with organ-specific or cell-specific antibody. In such embodiments, the liposomes are targeted to and taken up selectively by the organ. In some embodiments, the engineered IL2 polypeptide or engineered IL2 fusion polypeptide as described herein is provided in the form of a rapid release formulation, in the form of an extended or sustained release formulation, in the form of an intermediate release formulation, or in the form of a depot preparation. In some embodiments, the engineered IL2 polypeptide or engineered IL2 fusion polypeptide described herein is administered topically.

The appropriate dosage of an engineered IL2 polypeptide or engineered IL2 fusion polypeptide (used alone or in combination with one or more other additional therapeutic agents) will depend on the type of disease or condition, the route of administration, body weight of the subject, severity and progression of the disease, whether the polypeptide is administered for preventive or therapeutic purposes, previous or concurrent therapeutic interventions, the subject's clinical history and response to the engineered IL2 polypeptide or engineered IL2 fusion polypeptide, and the discretion of the attending physician. The practitioner responsible for administration will be able to determine the concentration of active ingredient(s) in a composition and appropriate dosing for the subject to be treated. Various dosing schedules including but not limited to single or multiple administrations over various time-points, bolus administration, and pulse infusion are contemplated herein.

A single administration of an engineered IL2 polypeptide may range from about 50,000 IU/kg to about 1,000,000 IU/kg or more of the engineered IL2 polypeptide. This may be repeated several times a day (e.g., 2-4 times per day), for several days (e.g., 3-5 consecutive days) and then may be repeated one or more times following a period of rest (e.g., 7-14 days). Thus, a therapeutically effective amount may comprise only a single administration or many administrations over a period of time (e.g. about 10-30 individual administrations of about 600,000 IU/kg of IL2 each given over about a 5-20 day period). When administered in the form of a fusion polypeptide, a therapeutically effective of the engineered IL2 fusion polypeptide may be lower than a non-fusion engineered IL2 polypeptide (e.g., 10,000 IU/kg to about 600,000 IU/kg). Similarly, the engineered IL2 fusion polypeptide may be administered to the patient at one time or over a series of treatments as described above.

In certain embodiments, the daily dosage of the engineered IL2 polypeptide or engineered IL2 fusion polypeptide ranges from about 1 μg/kg to about 100 mg/kg or more. For repeated administrations over several days or longer, depending on the condition, the treatment may be sustained until a desired suppression of disease symptoms occurs (e.g., tumor shrinkage). In some embodiments, a single dose of an engineered IL2 polypeptide or engineered IL2 fusion polypeptide is in the range from about 0.005 mg/kg to about 10 mg/kg. In some embodiments, a dose may be about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Such doses may be administered intermittently, e.g., 2-3 times per day, every week, or every three weeks. An initial higher loading dose, followed by one or more lower doses may be administered. However, other dosage regimens may be useful.

The engineered IL2 polypeptides and engineered IL2 fusion polypeptides described herein may be used in an amount effective to achieve the intended purpose. For use to treat or prevent a disease condition, the engineered IL2 polypeptides or engineered IL2 fusion polypeptides, or pharmaceutical compositions thereof, are administered in a therapeutically effective amount. Determination of a therapeutically effective amount is within the capabilities of those of skill in the art, especially in light of the details provided herein.

For systemic administration, a therapeutically effective amount can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Administration to humans could readily be optimized by a person of ordinary skill in the art based on animal data. Dosage amount and interval may each be adjusted to provide plasma levels of engineered IL2 polypeptides and engineered IL2 fusion polypeptides which are sufficient to maintain therapeutic effect. Levels in plasma may be measured, for example, by HPLC.

### EXAMPLES

### Example 1

### Library Strategy to Identify IL2Rα-Reduced Binding Mutations

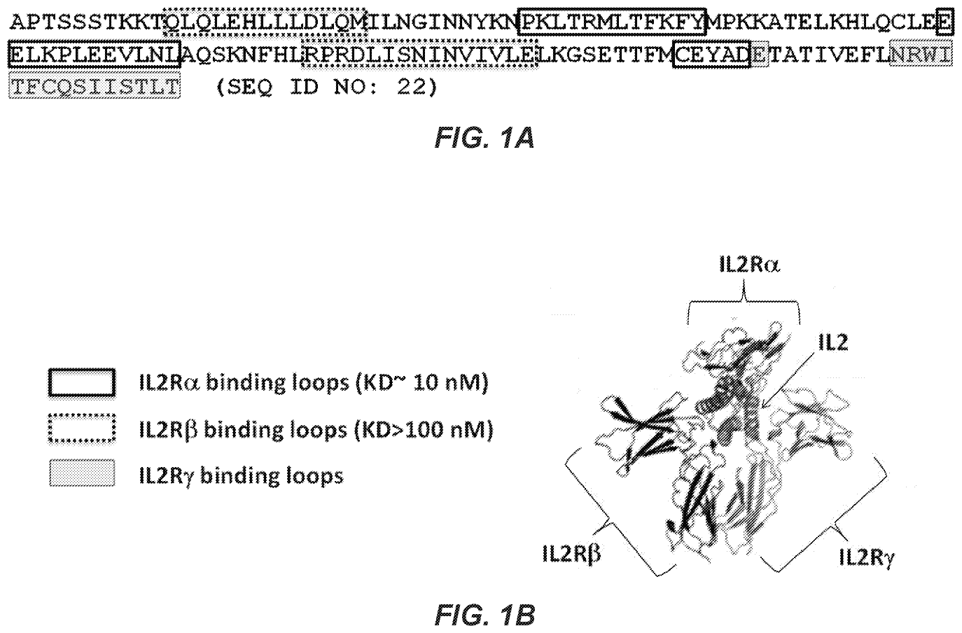

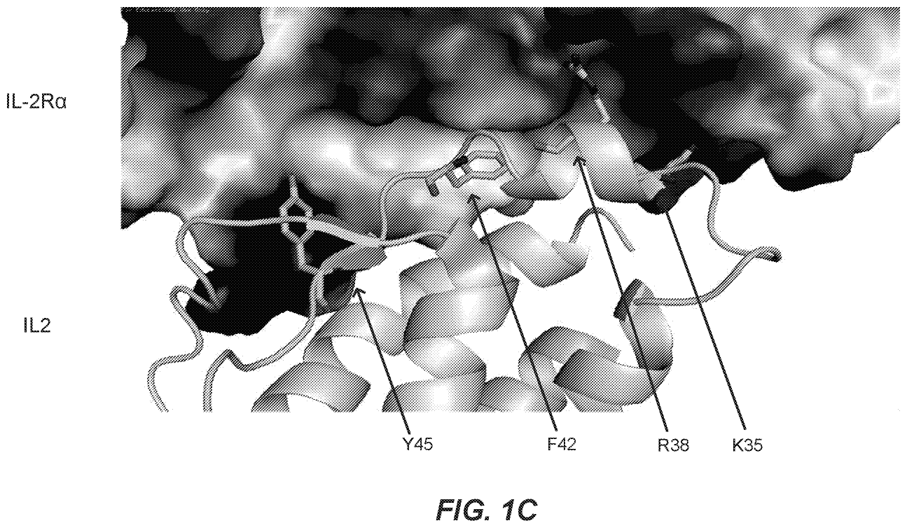

The libraries of IL2 mutations to identify IL2Rα-reduced binders were rationally designed based on the structural modeling of IL2 interactions with IL2Rα (FIGS. 1A & 1B). Briefly, K35, R38, F42 and Y45 residues of IL2 were identified as the important residues interacting with IL2Rα (FIG. 1C). Mutagenic oligos carrying two, three and four randomized mutations at these residues were used for library construction (Table 1).

TABLE 1
Rationally designed IL2Rα-reduced mutagenic libraries

| | | K35 | R38 | F42 | Y45 |
|---|---|---|---|---|---|
| Library 1 | 2 amino acid | NNS | NNS | F | Y |
| | mutagenesis | NNS | R | NNS | Y |
| | | NNS | R | F | NNS |
| | | K | NNS | NNS | Y |
| | | K | NNS | F | NNS |
| | | K | R | NNS | NNS |
| Library 2 | 3 amino acid | NNS | NNS | NNS | Y |
| | mutagenesis | NNS | R | NNS | NNS |
| | | K | NNS | NNS | NNS |
| Library 3 | 4 amino acid | K50%/ | R50%/ | A50%/ | Y50%/ |
| | mixed ratio | NNS50% | NNS50% | NNS50% | NNS50% |
| | mutagenesis | K70%/ | R70%/ | F70%/ | Y70%/ |
| | | NNS30% | NNS30% | NNS30% | NNS30% |

TABLE 2
Mutagenic oligo design for IL-2Rα-reduced binding
libraries

| | | SEQ |
|---|---|---|
| Oligo | | ID |
| # | Sequence | NO: |
| 1 | aac tac aag aac ccc NNS ctg acc NNS atg | 171 |
| | ctg acc ttc aag ttc tac atg cct aag aag | |
| | gcc acc | |
| 2 | aac tac aag aac ccc NNS ctg acc cgg atg | 172 |
| | ctg acc NNS aag ttc tac atg cct aag aag | |
| | gcc acc | |
| 3 | aac tac aag aac ccc NNS ctg acc cgg atg | 173 |
| | ctg acc ttc aag ttc NNS atg cct aag aag | |
| | gcc acc | |
| 4 | aac tac aag aac ccc aag ctg acc NNS atg | 174 |
| | ctg acc NNS aag ttc tac atg cct aag aag | |
| | gcc acc | |
| 5 | aac tac aag aac ccc aag ctg acc NNS atg | 175 |
| | ctg acc ttc aag ttc NNS atg cct aag aag | |
| | gcc acc | |
| 6 | aac tac aag aac ccc aag ctg acc cgg atg | 176 |
| | ctg acc NNS aag ttc NNS atg cct aag aag | |
| | gcc acc | |
| 7 | aac tac aag aac ccc NNS ctg acc NNS atg | 177 |
| | ctg acc NNS aag ttc tac atg cct aag aag | |
| | gcc acc | |
| 8 | aac tac aag aac ccc NNS ctg acc cgg atg | 178 |
| | ctg acc NNS aag ttc NNS atg cct aag aag | |
| | gcc acc | |
| 9 | aac tac aag aac ccc aag ctg acc NNS atg | 179 |
| | ctg acc NNS aag ttc NNS atg cct aag aag | |
| | gcc acc | |
| 10 | aac tac aag aac ccc AAG ctg acc CGG atg | 180 |
| | ctg acc GCC aag ttc TAC atg cct aag aag | |
| | gcc acc | |
| 11 | aac tac aag aac ccc AAG ctg acc CGG atg | 181 |
| | ctg acc GCC aag ttc TAC atg cct aag aag | |
| | gcc acc | |

Upper case nucleotide trimer is mixed at 50% WT and 50% NNS, or 70% WT and 30% NNS, where “N” refers to any nucleotide, and “S” refers to G or C. Three libraries were composed by multi-step PCR and overlapping PCR of above mutagenic oligos with WT IL2 sequence as template. Library 1 includes mutagenic oligos 1-6, library 2 includes mutagenic oligos 7-9, and library 3 includes mutagenic oligos 10-11 (Table 2). Those mutagenic libraries have been further modified to have in vitro transcription and translation signal at the N-terminus. A flag-tag sequence was also added to the C-terminus for selection and purification purpose.

### Example 2

### Selection and Identification of IL2Rα-Reduced Binding Clones

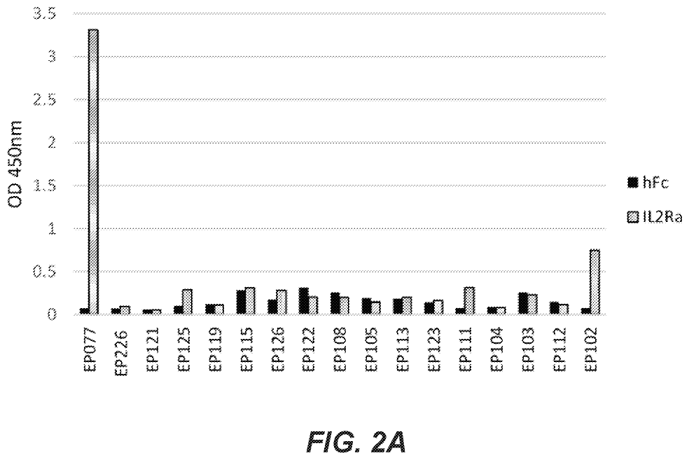

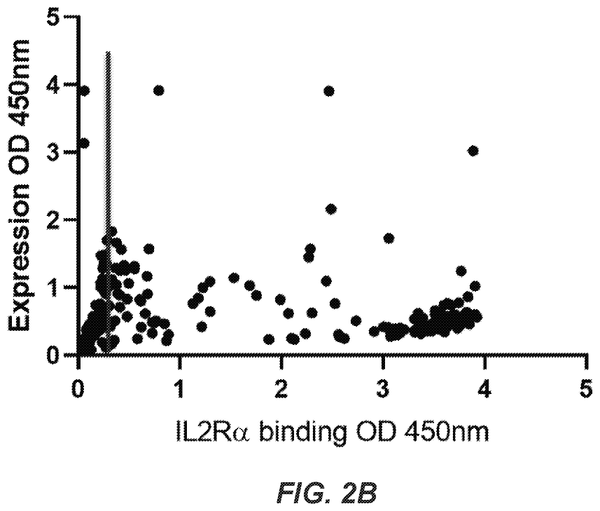

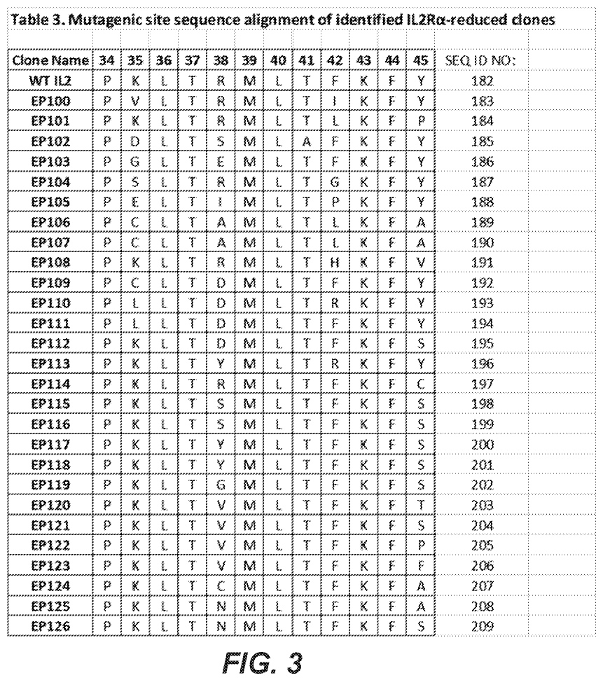

mRNA display technology was used to select IL2 mutants with IL2Rα-reduced binding from the three IL2 mutagenic libraries. Briefly, the DNA libraries were first transcribed into mRNA libraries and then translated into mRNA-IL2 mutant fusion libraries by covalent coupling through a puromycin linker. The libraries were purified and converted to mRNA/cDNA fusion libraries. The fusion libraries were counter-selected with human and mouse IgGs (negative proteins) to remove nonspecific binders, then counter-selected against IL2Rα three times. Library flow through (unbound molecules) was collected and PCR was performed to recover the IL2Rα unbound molecules followed by gel purification. The recovered pool was subcloned into pET22b vector and expressed in E. coli Rosetta II strands. The supernatant of individual clones was tested in IL2Rα binding ELISA. FIG. 2A shows the ELISA results of supernatant of selected IL2 clones to IL2Rα. FIG. 2B shows the clone expression plotted against IL2Rα binding with supernatant. FIG. 3 shows a sequence alignment of the clones identified with IL2Rα-reduced binding.

### Example 3

### Binding Kinetics Analysis of IL2Rα-Reduced Binding Clones

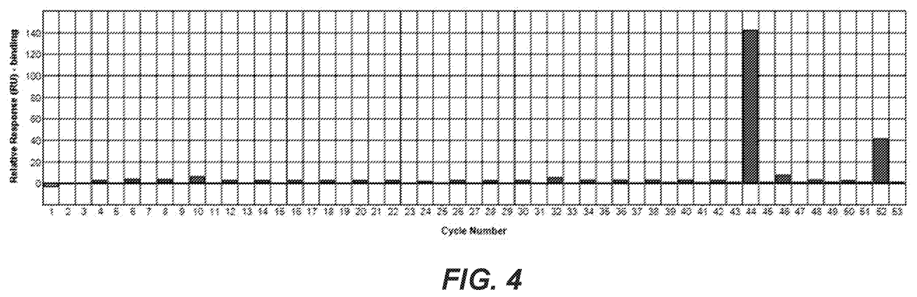

The binding kinetics of IL2Rα-reduced binding clones to IL2Rα was assessed utilizing SPR technology with a Biacore T200, software version 2.0. For each cycle, 1 ug/mL of human IL2Rα was captured for 60 seconds at a flow rate of 10 uL/min on flow cell 2 in 1×HBSP buffer on a Protein A sensor chip. 100 nM of each HIS and Flag tag purified IL2 mutant was injected onto both the reference flow cell 1 and IL2Rα captured flow cell 2 for 150 seconds at a flow rate of 30 uL/min followed by washing for 300 seconds. The flow cells were then regenerated with Glycine pH 2.0 for 60 seconds at a flow rate of 30 uL/min. A HBSP+ buffer was included with each sample as a baseline control. The assay was set up in a 96-well format. The kinetics data was analyzed with Biacore T200 evaluation software 3.0. The specific binding response unit was derived from subtraction of binding to reference flow cell 1 from IL2Rα flow cell 2 and subtraction of buffer control. WT IL2 was included as control. Relative response (RU) was determined for each IL2Rα-reduced binding clone (FIG. 4, Table 3).

TABLE 3
Binding kinetics analysis of IL2Rα-
reduced binding clones

| | | | Relevant |
|---|---|---|---|
| | | | binding to |
| | Cycle | RU | WT IL2 |
| Clone | Number | (at 100 nM) | (%) |
| Buffer | 3 | −0.1 | 0 |
| WT IL2 | 44 | 142 | 100 |
| EP001 | 50 | 2.2 | 1.55 |
| EP003 | 52 | 40.8 | 28.73 |
| EP101 | 4 | 2.5 | 1.76 |
| EP120 | 6 | 3.6 | 2.54 |
| EP121 | 8 | 3.4 | 2.39 |
| EP125 | 10 | 6.1 | 4.29 |
| EP119 | 12 | 2.7 | 1.9 |
| EP115 | 14 | 2.4 | 1.69 |
| EP126 | 16 | 2.6 | 1.83 |
| EP117 | 18 | 2.5 | 1.76 |
| EP122 | 20 | 2.5 | 1.76 |
| EP108 | 22 | 2.4 | 1.69 |
| EP100 | 24 | 1.7 | 1.19 |
| EP110 | 26 | 2.6 | 1.83 |
| EP105 | 28 | 2.1 | 1.48 |
| EP113 | 30 | 2.6 | 1.83 |
| EP123 | 32 | 5.3 | 3.73 |
| EP111 | 34 | 2.8 | 1.97 |
| EP104 | 36 | 2.8 | 1.97 |
| EP103 | 38 | 2.9 | 2.04 |
| EP112 | 40 | 2.9 | 2.04 |
| EP102 | 42 | 2.7 | 1.9 |
| EP225 | 46 | 7.4 | 5.21 |
| EP226 | 48 | 3 | 2.11 |

### Example 4

### Library Strategy to Generate IL2Rβ Agonists

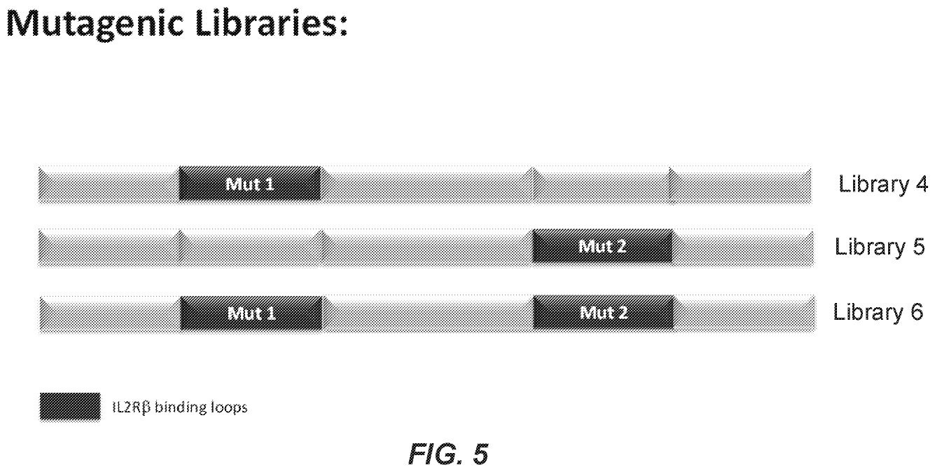

Engineered IL2Rβ binding agonists were created by rational IL2 mutagenic library design followed by selection with mRNA display technology platform. Briefly, two WT IL2 binding regions to IL2Rβ were identified: IL2Rβ binding region 1 “QLQLEHLLLDLQM” (SEQ ID NO: 67) and IL2Rβ binding region 2 “RPRDLISNINVIVLE” (SEQ ID NO: 68) from structural analysis. For production of mutagenic libraries including mutations to IL2Rβ binding region 1, IL2Rβ binding region 2, or IL2Rβ binding region 1 and IL2Rβ binding region 2, two mutagenic oligomers (Oligo 1, Oligo 2) encoding the sequences of these two regions were designed (Table 4). For Oligo 1 and Oligo 2 sequences, each codon trimer with nucleotides shown as lower-case letters was a mixture of 50% WT and 50% NNS (with “N” referring to any nucleotide, and “S” referring to G or C). Additional oligomers (Oligo 3 to Oligo 12) coding WT IL2 sequences were designed from WT region for mutagenic library assembly (Table 4). Three mutagenic libraries were constructed using these oligos (FIG. 5). Library 4 was constructed using an overlapped PCR strategy with mutagenic oligo 1 and oligos 3-12. Library 5 was constructed using an overlapped PCR strategy with mutagenic oligo 2 and oligos 3-9, 11, and 12. Library 6 was constructed using an overlapped PCR strategy with mutagenic oligos 1 and 2 and oligos 3-8, 11, and 12. The three mutagenic libraries were further modified to have an in vitro transcription and translation signal at N-terminus and a Flag-tag at the C-terminus for selection with mRNA display.

TABLE 4
Mutagenic oligo design for IL-2R agonist libraries

| | | SEQ |
|---|---|---|
| Oligo | | ID |
| # | Sequence | NO: |
| 1 | AGT TCT ACA AAG AAA ACA cag cta caa ctg | 69 |
| | gag cat tta ctg ctg gat tta cag atg ATT | |
| | TTG AAT GGA ATT AAT | |
| 2 | AGC AAA AAC TTT CAC TTA aga ccc agg gac | 70 |
| | tta atc agc aat atc aac gta ata gtt ctg | |
| | gaa CTA AAG GGA TCT GAA ACA | |
| 3 | AATTACTATTTACAATTACAATGGCTAGCGCACCTACTTC | 71 |
| | AAGTTCTACAAAGAAAACA | |
| 4 | CTTGGGCATGTAAAACTTAAATGTGAGCATCCTGGTGAG | 72 |
| | TTTGGGATTCTTGTAATTATTAATTCCATTCAAAAT | |
| 5 | AAGTTTTACATGCCCAAGAAGGCCACAGAACTGAAACAT | 73 |
| | CTTCAGTGTCTAGAAGAAGAACTCAAACCTC | |
| 6 | TAAGTGAAAGTTTTTGCTTTGAGCTAAATTTAGCACTTCC | 74 |
| | TCCAGAGGTTTGA GTTCTTC | |
| 7 | CTAAAGGGATCTGAAACAACATTCATGTGTGAATATGCT | 75 |
| | GATGAGACAGCAACCATTGTAGAATTTCTGAACAGA | |
| 8 | AGATGGTGCAGCCACAGTTCGAGTCAGTGTTGAGATGAT | 76 |
| | GCTTTGACAAAAGGTAATCCATCTGTTCAGAAATTCTAC | |
| 9 | AGTTCTACAAAGAAAACACAGCTACAACTGGAGCATTTA | 77 |
| | CTGCTGGATTTACAGATGATTTTGAATGGAATTAAT | |
| 10 | AGCAAAAACTTTCACTTAAGACCCAGGGACTTAATCAG | 78 |
| | CAATATCAACGTAATAGTTCTGGAACTAAAGGGATCTG | |
| | AAACA | |
| 11 | AAGTTTTACATGCCCAAG | 79 |
| 12 | TAAGTGAAAGTTTTTGC | 80 |

### Example 5

### Selection and Identification of IL2Rβ Agonist Clones

An mRNA display technology platform was used for the identification of IL2Rβ agonists from three IL2 mutagenic libraries. The DNA libraries were first transcribed into mRNA libraries and then translated into mRNA-IL2 mutant fusion libraries by covalent coupling through a puromycin linker. The libraries were then purified and converted to mRNA/cDNA fusion libraries (see, e.g., U.S. Pat. No. 6,258,558, hereby incorporated by reference). The fusion libraries were first counter selected with human IgGs (negative proteins) to remove nonspecific binders, then counter selected to remove IL2Rα binders, followed by selection against recombinant IL2Rβ/Fc protein captured on Protein G magnetic beads. The IL2Rβ binders were recovered and enriched by PCR amplification. A total of five rounds of selections were executed to generate highly enriched engineered IL2 mutants binding to IL2Rβ.

Following five rounds of selection, enriched libraries were cloned into bacterial periplasmic expression vector pET22b and transformed into TOP10 competent E. coli cells. Each engineered IL2 molecule was engineered to have a C-terminus flag and 6×HIS tag for purification and assay detection. Clones from TOP10 cells were pooled and the miniprep DNA were prepared and subsequently transformed into E. coli Rosetta II strain for expression. Single clones were picked, grown and induced with 0.25 mM IPTG in 96-well plates for expression. The supernatant was collected after 16 to 24 hours induction at 30° C. for assays to identify binders.

Supernatants containing engineered IL2 mutants were assessed with sandwich ELISA assay to screen for expression. Briefly, an anti-HIS tag antibody (R&D Systems) was immobilized in a 96-well plate at a final concentration of 2 μg/mL in 1×PBS in a total volume of 50 μL per well. The plate was incubated overnight at 4° C. followed by blocking with 200 μL of superblock per well for 1 hour. 100 μL of 1:10 1×PBST diluted supernatant was added to each well and incubated for 1 hour with shaking. The expression level of engineered IL2 mutant was detected by adding 50 μl of anti-Flag HRP diluted at 1:5000 in 1×PBST for one hour. In between each step, the plate was washed three times with 1×PBST using a plate washer. The plate was then developed with 50 μL of TMB substrate for 5 minutes and stopped by adding 50 of 2N sulfuric acid. The plate was read at OD₄₅₀ nm using a Biotek plate reader and the data was analyzed with Prism 8.1 software.

Single clones were next screened for IL2Rβ binding. IL2Rβ binding screening ELISA was developed for the identification of individual engineered IL2 mutant. Briefly, 96-well plate was immobilized with human Fc and human IL2Rβ, respectively, at a final concentration of 2 μg/mL in 1×PBS in total volume of 50 per well. The plate was incubated overnight at 4° C. followed by blocking with 200 of superblock per well for 1 hour. 100 μl of supernatant was added to both Fc and IL2Rβ immobilized wells and incubated for 1 hour with shaking. The engineered IL2 mutant binding was detected by adding 50 μL of anti-Flag HRP diluted at 1:5000 in 1×PBST. In between each step, the plate was washed three times with 1×PBST in a plate washer. The plate was then developed with 50 μL of TMB substrate for five minutes and stopped by adding 50 μL of 2N sulfuric acid. The plate was read at OD450 nm using a Biotek plate reader and the binding and selectivity was analyzed. The correlation of expression and IL2Rβ binding was plotted with Prism 8.1 software.

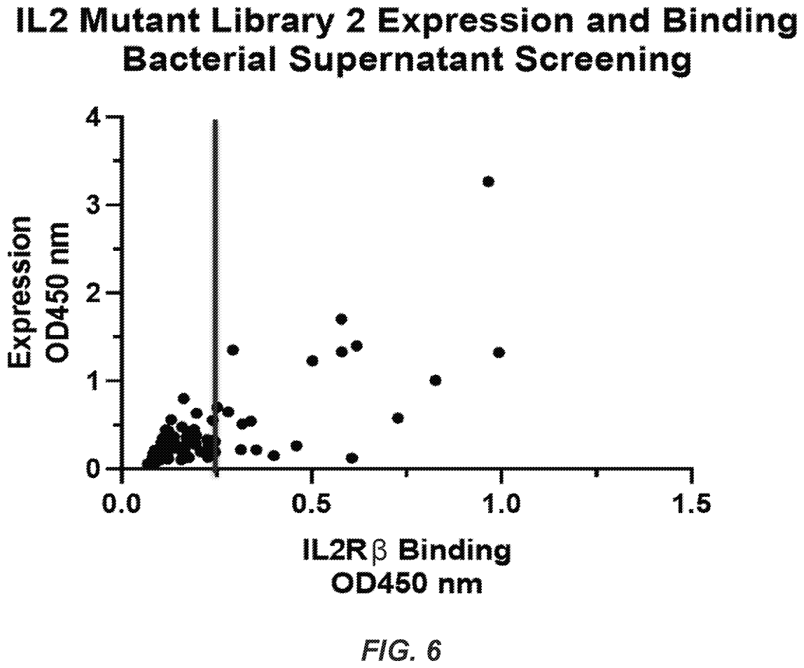

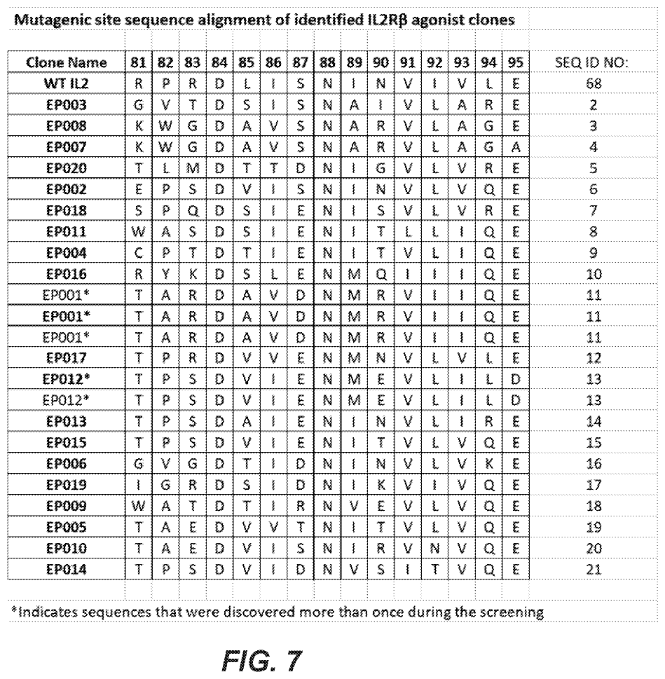

High IL2Rβ-binding clones (shown to the right of the vertical OD450 nm cutoff) were identified from library 5 for further characterization as engineered IL2Rβ agonists (FIG. 6). The IL2Rβ-binding activity of the clones generally correlated with expression level of the clones. Multiple sequence alignment of the IL2Rβ binding region 2 revealed both highly conserved and highly varied amino acids as compared to IL2 WT, as well as clone sequences that were identified multiple times independently (FIG. 7). No specific IL2Rβ-binding clones were identified from libraries 4 and 6.

### Example 6

### Production of IL2Rβ Agonist Clones in E. Coli and Mammalian Cells

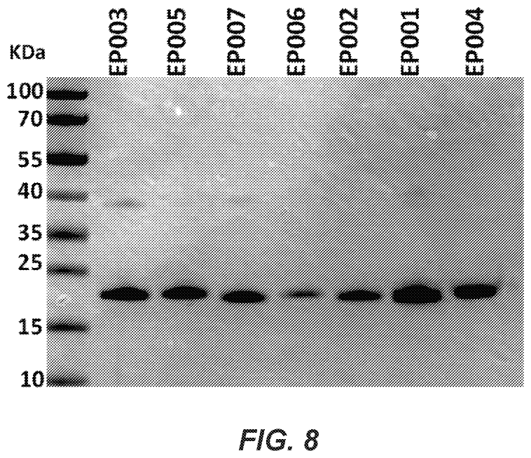

For production of the IL2Rβ agonists in E. coli, the glycerol stock of each engineered agonist clone was inoculated into TB medium for overnight growth. The next day, cells from the overnight culture were inoculated into TB medium and grown to a cell density of OD₆₀₀ between 0.6-0.8. IPTG was added to a final concentration of 1 mM to induce the expression during culture at 30 degrees C. overnight. The supernatant was collected by centrifugation. The engineered agonists were purified by Ni-Sepharose (GE Healthcare) affinity column according to the manufacturer's protocol. The purity of the engineered agonists was further improved by Flag-tag affinity column purification (Sigma). The agonists were each concentrated and loaded to a Sephadex 200 Increase 10/300 GL column in AKTA for size exclusion chromatographic column purification. The high homogeneous monomeric peak fractions of the agonists were each pooled and concentrated. Endotoxin was further removed using endotoxin removal resin (Pierce) according to the standard protocol. The final endotoxin level was less than 10 EU/mg. Protein purity was confirmed by LC-MS spectrometry analysis and SDS gel (FIG. 8). The proteins were each stored in 1×PBS buffer for binding and functional analysis.

For production of the IL2Rβ agonists in mammalian cells, the DNA sequence corresponding to the amino-acid sequence was codon optimized, synthesized and subcloned into pCDNA3.4 (Invitrogen). Each engineered IL2 polypeptide was expressed transiently in ExpiHEK293-F cells in free style system (Invitrogen) according to standard protocol. The cells were grown in the above conditions for seven days before harvesting. The supernatant was collected by centrifugation and filtered through a 0.2 μm PES membrane. The agonists were first purified by Ni Sepharose Excel resin column (GE Healthcare) and buffer exchanged to PBS pH 7.4+300 mM NaCl (total) with 7 k Da Zeba columns. Each polypeptide was then concentrated to 1 mL and purified by a Superdex 200 Increase 10/300 GL column (GE Healthcare) to homogeneity. The monomeric peak fractions were pooled and concentrated. The final purified protein contained less 10 EU/mg endotoxin. The IL2 polypeptide identify was confirmed by LC-MS spectrometry analysis and purity analyzed by SDS gel. The proteins were stored in 1×PBS/300 nM NaCl buffer for binding, functional and mechanism analysis.

### Example 7

### Binding Kinetics Analysis of IL2Rβ Agonist Clones Using Surface Plasmon Resonance

The binding kinetics of E. coli produced WT IL2 and EP001-EP007 were assessed by surface plasmon resonance technology with Biacore T200 for engineered IL2 polypeptides produced in E. coli cells and engineered IL2 polypeptides produced in mammalian cells. The assay was run with Biacore T200 control software version 2.0. For each cycle, 1 μg/mL of human IL2Rβ or IL2Rα was captured for 60 seconds at flow rate of 10 μL/min on flow cell 2 in 1×HBST buffer on Protein A sensor chip. Two-fold serial diluted HIS tag purified engineered IL2 mutant was injected onto both reference flow cell 1 and IL2Rβ or IL2Rα captured flow cell 2 for 150 seconds at flow rate of 30 μL/min followed by washing for 300 seconds. The flow cells were then regenerated with Glycine pH 2 for 40 seconds at a flow rate of 30 μL/mins. Eight concentration points from 0 to 100 nM were assayed for each IL2Rβ agonist clone 96-well plate format. The kinetics data was analyzed with Biacore T200 evaluation software 3000. The specific binding response unit was derived from subtraction of binding to reference flow cell 1 from target flow cell 2.

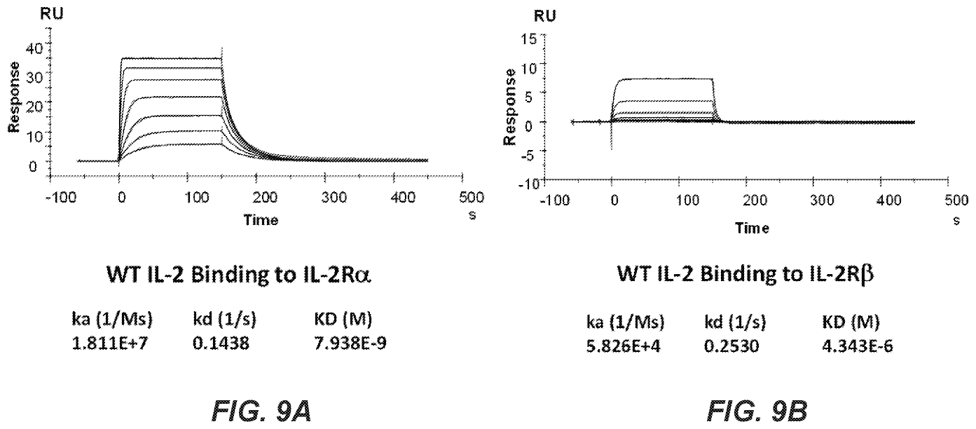

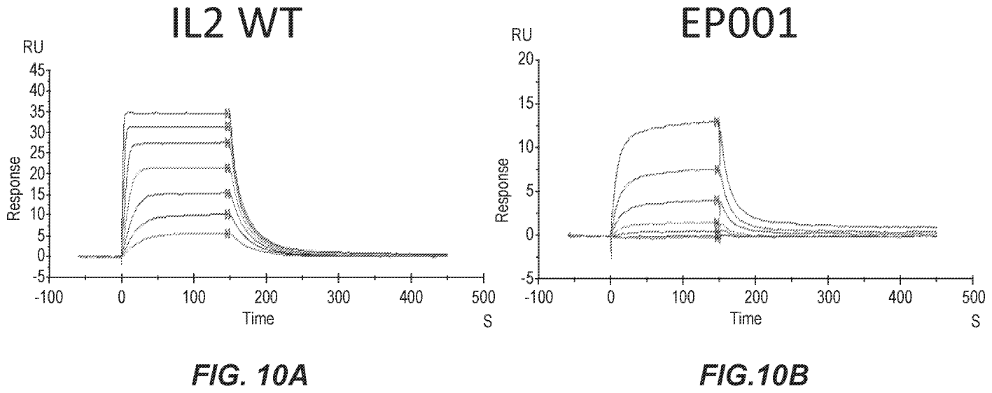

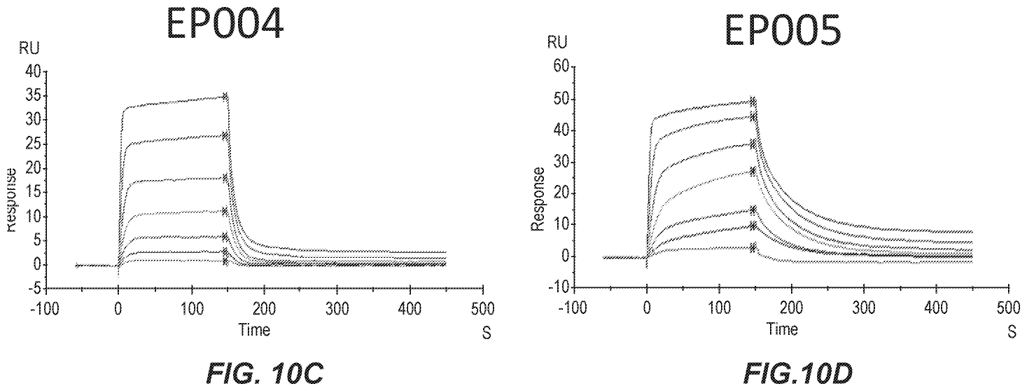

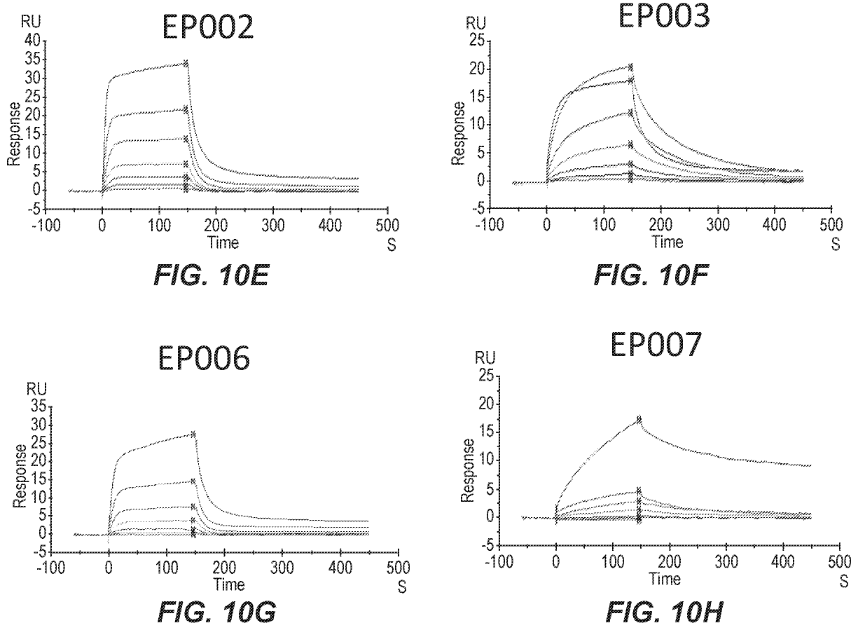

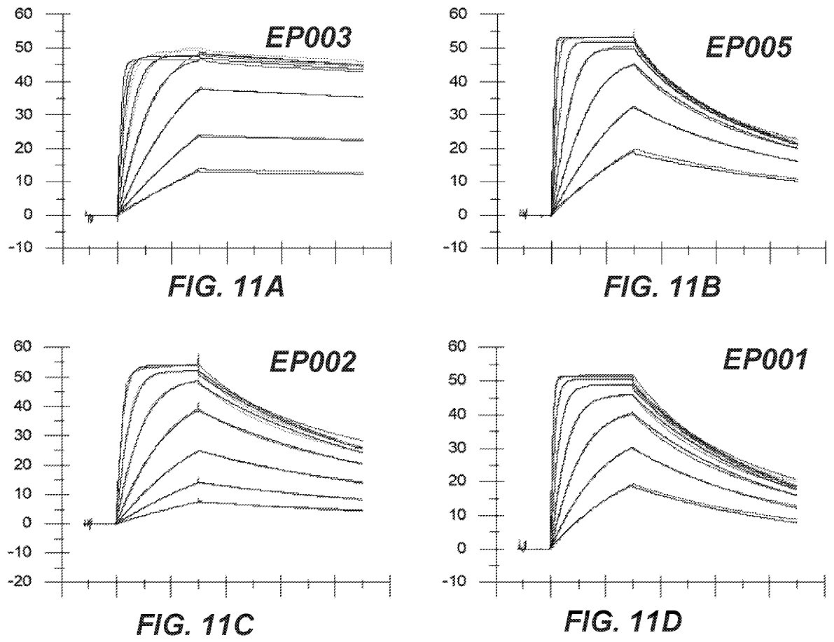

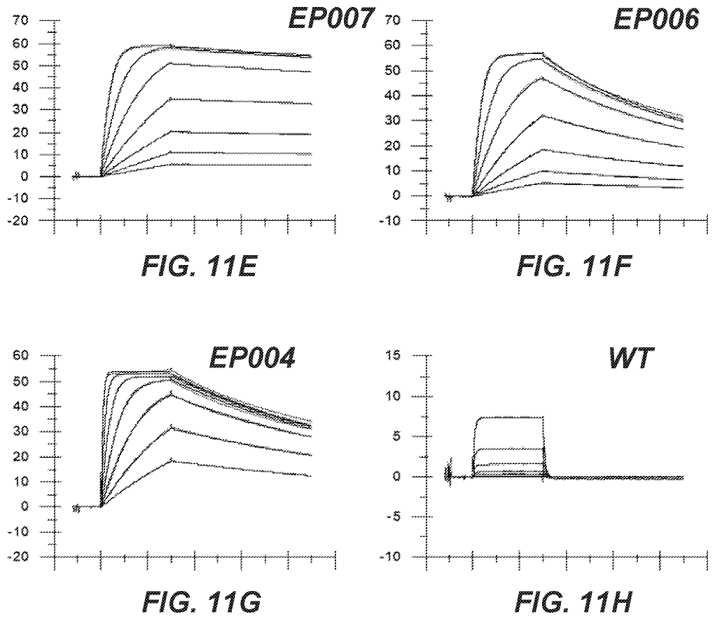

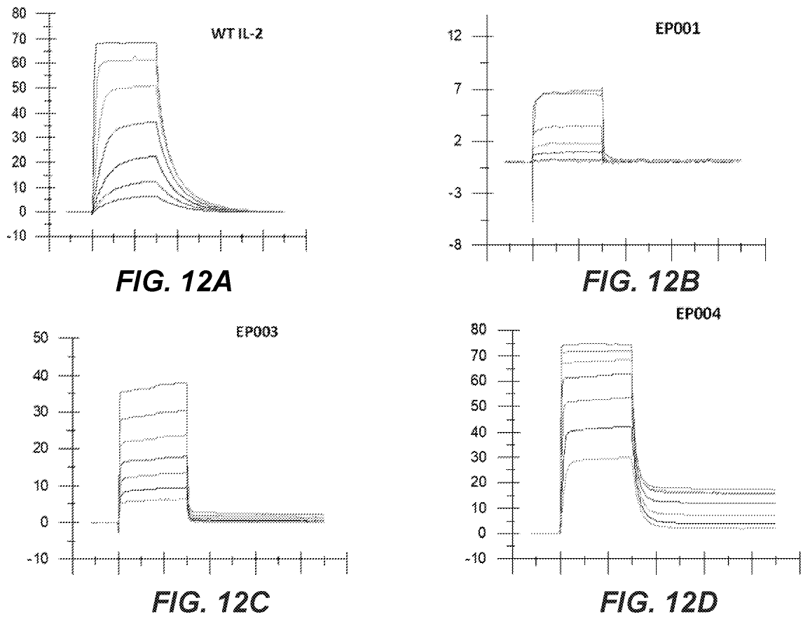

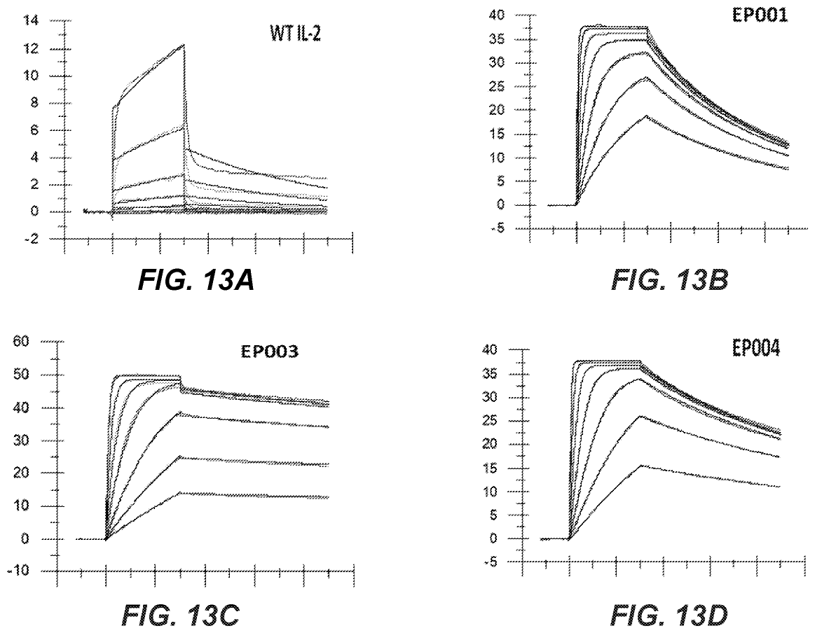

WT IL2 was used to validate the binding protocols and was included in each run as a control (FIGS. 9A & 9B). Representative sensorgrams of binding kinetics of E. coli and mammalian produced IL2Rβ agonist binding to IL2Rα (FIGS. 10A-10H for E. coli produced, FIGS. 12A-12D for mammalian produced) and IL2Rβ (FIGS. 11A-11H for E. coli produced, FIGS. 13A-13D for mammalian produced) are shown. Binding kinetics for E. coli-produced IL2 (Table 5) and mammalian-produced IL2 (Table 6) are summarized.

IL2Rβ agonists EP001, EP006, and EP007 demonstrated no detectable IL2Rα binding or a significant decrease (greater than a twenty-fold decrease) in IL2Rα binding, but had a significant increase in IL2Rβ binding as compared to WT IL2 (Table 5, 6). In contrast, the IL2Rβ agonists EP002, EP003, EP004, and EP005 did not demonstrate a significant decrease in IL2Rα (less than a twenty-fold decrease) as compared to wild-type IL2, but had a significant increase in IL2Rβ binding as compared to WT IL2 (Tables 5, 6).

TABLE 5
Summary of IL2R binding kinetics of E.coli-produced IL2Rβ agonist clones

| | | | | | Relative activity | |
|---|---|---|---|---|---|---|
| | IL2α | IL2Rβ | | | IL2Rα | IL2Rβ |
| | KD* | Kon | Koff | KD | (KD: | (KD: |
| Clones | (M) | (1/Ms) | (1/s) | (M) | fold) | fold) |
| WT IL2 | 9.51E−09 | 5.83E+04 | 0.253 | 4.34E−06 | — | — |
| EP001 | ND | 3.26E+06 | 0.003646 | 1.12E−09 | NA | 3888 |
| EP002 | 1.01E−07 | 1.15E+06 | 0.002734 | 2.37E−09 | −10.6 | 1834 |
| EP003 | 2.14E−08 | 2.18E+06 | 2.70E−04 | 1.23E−10 | −2.3 | 35194 |
| EP004 | 4.05E−08 | 3.05E+06 | 0.001892 | 6.20E−10 | −4.3 | 7007 |
| EP005 | 1.26E−08 | 6.10E+06 | 0.005397 | 8.85E−10 | −1.3 | 4909 |
| EP006 | 4.81E−07 | 8.19E+05 | 0.002541 | 3.10E−09 | −50.6 | 1399 |
| EP007 | ND | 7.09E+05 | 2.63E−04 | 3.70E−10 | NA | 11728 |

TABLE 6
Summary of IL2R binding kinetics of mammalian-produced IL2Rβ agonist clones

| | | | | | Relative activity | |
|---|---|---|---|---|---|---|
| | IL2α | IL2Rβ | | | IL2Rα | IL2Rβ |
| | KD* | Kon | Koff | KD | (KD: | (KD: |
| Clones | (M) | (1/Ms) | (1/s) | (M) | fold) | fold) |
| WT IL2 | 1.35E−08 | 3.26E+03 | 0.00329 | 1.01E−06 | — | — |
| EP001 | ND | 5.61E+06 | 0.00417 | 7.43E−10 | NA | 1359 |
| EP003 | 3.02E−07 | 2.59E+06 | 0.000376 | 1.45E−10 | −22.4 | 6966 |
| EP004 | 2.88E−08 | 5.18E+06 | 2.15E−03 | 4.14E−10 | −2.1 | 2422 |

### Example 8

### IL2Rβ Agonist P-STAT5 Activation in Human PBMCs

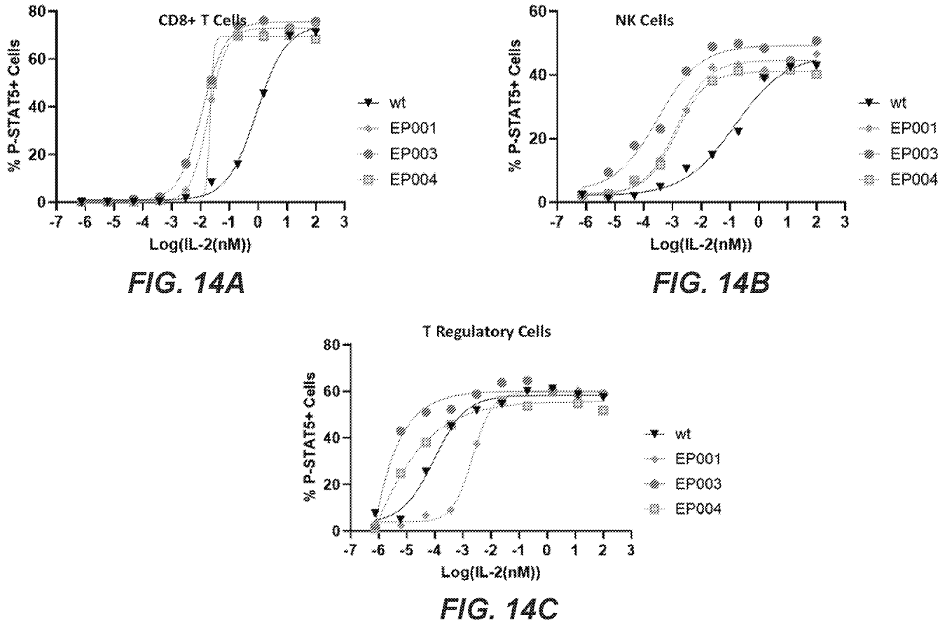

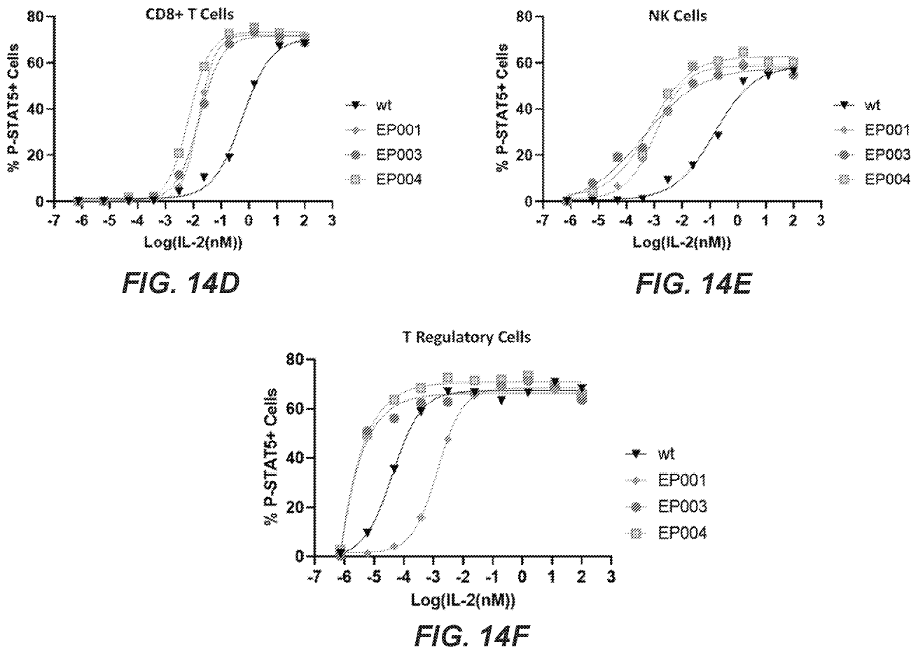

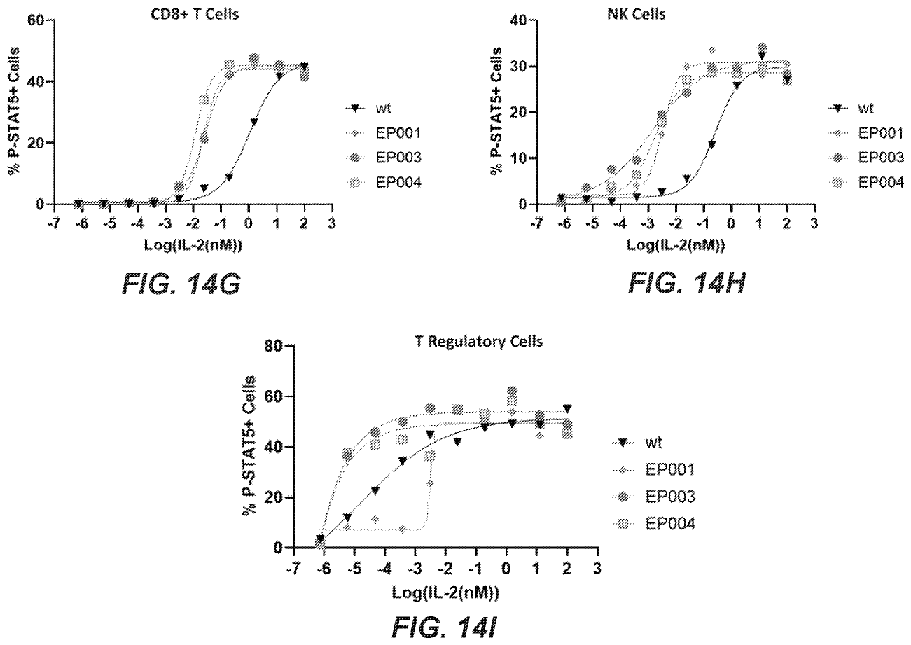

Human PBMCs were isolated from peripheral blood of three separate donors and plated at 250,000 cells/well in a 96-well plate in 75 μL of media. Cells were rested 1 hr at 37° C. Cells were stimulated with human WT IL2 and engineered His-Flag tagged IL2 at 4× concentration in 25 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIGS. 14A-14C for blood donor 1, FIGS. 14D-14F for blood donor 2, and FIGS. 14G-14I for blood donor 3). EC50 values for P-STAT5 activation were determined using Prism software (Table 7).

TABLE 7
Summary of P-STAT5 activation of
human CD8+ T cells, NK cells and Tregs

| | | EC50 (nM) | | | |
|---|---|---|---|---|---|
| | | WT | EP001 | EP003 | EP004 |
| Blood | CD8+ T Cells | 8.9E−01 | 1.9E−02 | 1.1E−02 | 2.1E−02 |
| Donor 1 | NK Cells | 1.5E−01 | 1.4E−03 | 3.1E−04 | 1.4E−03 |
| | T Regulatory | 9.5E−05 | 2.2E−03 | <1.0E−5 | <1.0E−5 |
| | Cells | | | | |
| Blood | CD8+ T Cells | 5.6E−01 | 1.6E−02 | 1.7E−02 | 7.1E−03 |
| Donor 2 | NK Cells | 1.5E−01 | 1.4E−03 | 4.2E−04 | 7.1E−04 |
| | T Regulatory | 4.2E−05 | 1.4E−03 | <1.0E−5 | <1.0E−5 |
| | Cells | | | | |
| Blood | CD8+ T Cells | 1.1E+00 | 2.3E−02 | 2.6E−02 | 1.2E−02 |
| Donor 3 | NK Cells | 2.6E−01 | 3.3E−03 | 1.4E−03 | 1.8E−03 |
| | T Regulatory | 2.0E−05 | 3.2E−03 | <1.0E−5 | <1.0E−5 |
| | Cells | | | | |

### Example 9

### Rational Generation of IL2Rβ Agonist Back-Mutation Clones

Rationally designed IL2Rβ agonist back-mutation strategy was carried out to create a range of IL2Rβ agonist candidate mutations. EP001 contains R81T, P82A, L85A, I86V, S87D, I89M, N90R, V93I and L94Q mutations. Four back mutations to WT IL2 were designed for each candidate. The I86 and I89 were back-mutated to 861 and 891 of WT IL2 for all mutations. A systemic back-mutation was then applied to the other two residues in combination with 861 and 891. A total of 21 back-mutation combinations were designed and the mutations were created by site directed mutagenesis using EP001 as template (Table 8). IL2Rβ agonist back-mutation clones were sequence verified after mutagenesis.

TABLE 8
Back mutations introduced into EP0001

| | 81 | 82 | 85 | 86 | 87 | 89 | 90 | 93 | 94 |
|---|---|---|---|---|---|---|---|---|---|
| WT IL2 | R | P | L | I | S | I | N | V | L |
| EP001 | T | A | A | V | D | M | R | I | Q |
| EP242 | T | A | A | I | S | I | R | V | Q |
| EP243 | R | P | A | I | D | I | R | I | Q |
| EP244 | R | A | L | I | D | I | R | I | Q |
| EP245 | R | A | A | I | S | I | R | I | Q |
| EP246 | R | A | A | I | D | I | N | I | Q |
| EP247 | R | A | A | I | D | I | R | V | Q |
| EP248 | R | A | A | I | D | I | R | I | L |
| EP249 | T | P | L | I | D | I | R | I | Q |
| EP250 | T | P | A | I | S | I | R | I | Q |
| EP251 | T | P | A | I | D | I | N | I | Q |
| EP252 | T | P | A | I | D | I | R | V | Q |
| EP253 | T | P | A | I | D | I | R | I | L |
| EP254 | T | A | L | I | S | I | R | I | Q |
| EP255 | T | A | L | I | D | I | N | I | Q |
| EP256 | T | A | L | I | D | I | R | V | Q |
| EP257 | T | A | L | I | D | I | R | I | L |
| EP258 | T | A | A | I | S | I | N | I | Q |
| EP260 | T | A | A | I | S | I | R | I | L |
| EP261 | T | A | A | I | D | I | N | V | Q |
| EP262 | T | A | A | I | D | I | N | I | L |
| EP263 | T | A | A | I | D | I | R | V | L |

### Example 10

### Characterization of IL2Rβ Agonist-Back-Mutation Clones

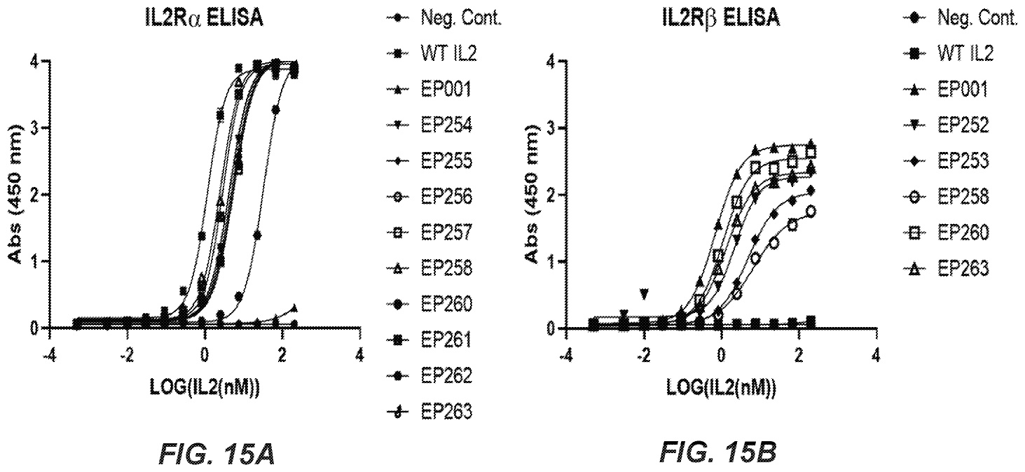

EP001 back-mutation clones were characterized for their binding activities to IL2Rβ and IL2Rα receptors by ELISA. Briefly, 384 well plate was immobilized with human IL2Rα and IL2Rβ Fc fusion proteins at a final concentration of 2 ug/mL in 1×PBS in total volume of 25 uL per well. The plate was incubated overnight at 4 C and blocked with 80 uL of superblock per well for 1 hour. The purified EP001 back-mutation clones were serially diluted from 100 nM to 0 nM. Each dilution was added to IL2Rα or IL2Rβ wells in parallel in duplicates. The IL2 mutant binding was detected by adding 25 uL of anti-Flag HRP diluted at 1:5000 in 1×PBST. In between each step, the plate was washed three times with 1×PBST using a plate washer. The plate was then developed with 25 uL of TMB substrate for five minutes and stopped by adding 25 ul of 2N sulfuric acid. The plate was read at OD450 nm Biotek plate reader and the EC50 was analyzed with Prism 8.1 software to generate EC50 values (FIG. 15A for IL2Rα, and FIG. 15B for IL2Rβ, summarized in Table 9).

TABLE 9
Binding activity of back-mutation clones

| | IL2Rα | IL2Rβ |
|---|---|---|
| | Binding | Binding |
| Clone | EC50 (nM) | EC50 (nM) |
| WT IL2 | 1.2 | ND |
| EP001 | 259.0 | 0.7 |
| EP003 | 538.5 | 1.8 |
| EP242 | 8.7 | 5.7 |
| EP243 | 6.7 | 217.1 |
| EP244 | 10.2 | 2830.0 |
| EP245 | 4.8 | 122.8 |
| EP246 | 7.0 | 269.0 |
| EP247 | 8.3 | 4.7 |
| EP248 | 13.4 | 188.0 |
| EP249 | 20.2 | 576.1 |
| EP250 | 13.2 | 23.4 |
| EP251 | 16.8 | 2.5 |
| EP252 | 13.0 | 2.2 |
| EP253 | 48.5 | 4.8 |
| EP254 | 4.3 | 556.1 |
| EP255 | 4.3 | 359.5 |
| EP256 | 5.3 | 361.9 |
| EP257 | 5.6 | ND |
| EP258 | 2.5 | 6.5 |
| EP260 | 5.4 | 1.1 |
| EP261 | 2.9 | 124.5 |
| EP262 | 32.1 | 53.2 |
| EP263 | 4.9 | 1.3 |

### Example 11

### Binding Kinetics of IL2Rβ Agonist Back-Mutation Clones Using Surface Plasmon Resonance

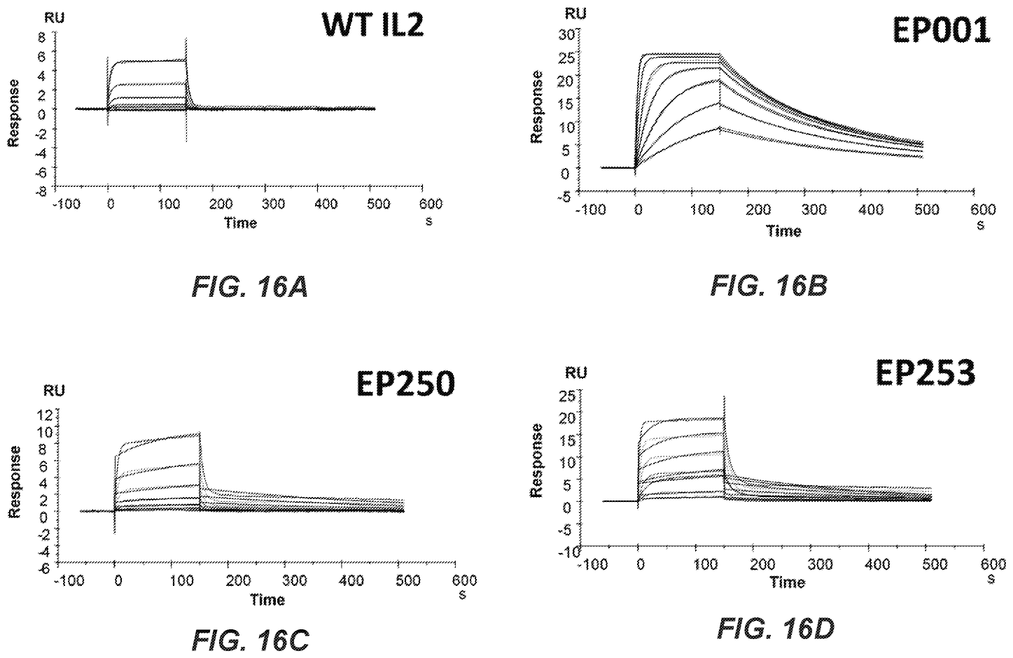

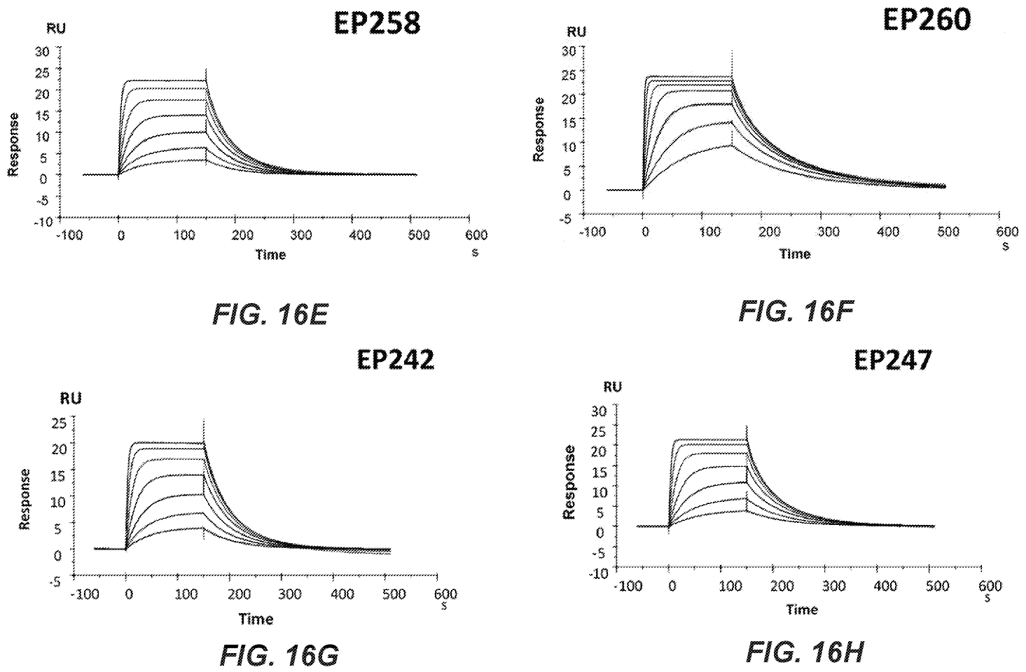

Binding kinetics analysis of EP001 back-mutation clones have been assessed by SPR technology with a Biacore T200. The assay was run with Biacore T200 control software version 2.0. For each cycle, 1 ug/mL of human IL2Rβ was captured for 60 seconds at flow rate of 10 uL/min on flow cell 2 in 1×HBSP buffer on Protein A sensor chip. 100 nM of HIS and Flag tag purified each IL2 mutant was 2-fold serial diluted and injected onto both reference flow cell 1 and IL2Rβ captured flow cell 2 for 150 seconds at flow rate of 30 uL/min followed by washing for 300 seconds. The flow cells were then regenerated with Glycine pH2 for 60 seconds at a flow rate of 30 uL/min. The assay was set up with 8 serial diluted concentration points in 96-well format. The kinetics data was analyzed with Biacore T200 evaluation software 3.0. The specific binding response unit was derived from subtraction of binding to reference flow cell 1 from target flow cell 2 (FIGS. 16A-16F, Table 10).

TABLE 10
Summary of binding kinetics forback-mutation clones.

| | ka (1/Ms) | kd (1/s) | KD (M) |
|---|---|---|---|
| EP001 | 3.86E+06 | 5.89E−03 | 1.52E−09 |
| EP242 | 3.36E+06 | 2.41E−02 | 7.16E−09 |
| EP247 | 4.20E+06 | 3.04E−02 | 7.23E−09 |
| EP252 | NA | NA | 9.110E−9* |
| EP253 | NA | NA | 2.947E−8* |
| EP258 | 3.42E+06 | 3.01E−02 | 8.81E−09 |
| EP260 | 1.06E+07 | 2.24E−02 | 2.11E−09 |
| EP263 | 6.84E+06 | 1.91E−02 | 2.79E−09 |

(NA: data not available; *KD measured from steady state affinity fitting)

### Example 12

### Engineered IL2Rβ Agonists with Reduced IL2Rα Activity

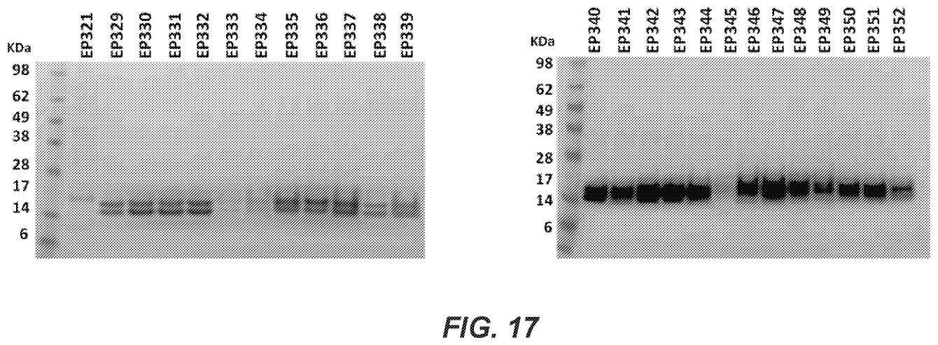

IL2 mutations with potentially reduced or eliminated IL2Rα binding generated through mRNA library selection and screen were first expressed in E. coli and purified by Ni-Sepharose (GE Healthcare) affinity column and Flag tag affinity column purification (Sigma) according to the manufacturer's protocol. The IL2 mutations with significant reduced IL2Rα binding activities confirmed by both Biacore SPR binding and ELISA binding were then selected for generating IL2Rβ agonists with reduced IL2Rα binding activities. Briefly, mutations were introduced by site directed mutagenesis technologies to IL2Rβ agonist constructs in pCDNA3.4 mammalian expression vector and confirmed by DNA sequence analysis (Table 11). Each engineered IL2 polypeptide was expressed transiently in ExpiHEK293-F cells in free style system (Invitrogen) according to standard protocol. The cells were grown in above conditions for five days before harvesting. The supernatant was collected by centrifugation and filtered through a 0.2 μm PES membrane. The agonists were first purified by Ni Sepharose Excel resin column (GE Healthcare) and further purified by a Superdex 200 Increase 10/300 GL column (GE Healthcare) to more than 95% homogeneity (FIG. 17). The final purified proteins have less than 10 EU/mg endotoxin. The proteins were stored in 1×PBS buffer for binding, functional and mechanism analysis.

TABLE 11
Engineered IL2Rβ agonists with IL2Rα
reduced-binding mutations

| Engineered | | | |
|---|---|---|---|
| IL2Rβ | Parental | | |
| agonist with | IL2Rα- | | Parental |
| IL2Rα | Reduced | | IL2Rβ |
| Reduced | Binding | IL2Rα | Agonist |
| Activity | Clones | Mutations | Clone |
| EP329 | EP103 | K35G/R38E | EP260 |
| EP330 | | K35G/R38E | EP258 |
| EP331 | | K35G/R38E | EP252 |
| EP332 | | K35G/R38E | EP253 |
| EP333 | EP104 | K35S, F42G | EP260 |
| EP334 | | K35S, F42G | EP258 |
| EP335 | | K35S, F42G | EP252 |
| EP336 | | K35S, F42G | EP253 |
| EP337 | EP110 | K35L/R38D/F42R | EP260 |
| EP338 | | K35L/R38D/F42R | EP258 |
| EP339 | | K35L/R38D/F42R | EP252 |
| EP340 | | K35L/R38D/F42R | EP253 |
| EP341 | EP112 | R38D/Y45S | EP260 |
| EP342 | | R38D/Y45S | EP258 |
| EP343 | | R38D/Y45S | EP252 |
| EP344 | | R38D/Y45S | EP253 |
| EP345 | EP121 | R38V/Y45S | EP260 |
| EP346 | | R38V/Y45S | EP258 |
| EP347 | | R38V/Y45S | EP252 |
| EP348 | | R38V/Y45S | EP253 |
| EP349 | EP239 | F42A | EP260 |
| EP350 | | F42A | EP258 |
| EP351 | | F42A | EP252 |
| EP352 | | F42A | EP253 |

### Example 13

### Binding Kinetics Analysis of Engineered IL2Rα/IL2Rβ Clones Using Surface Plasmon Resonance

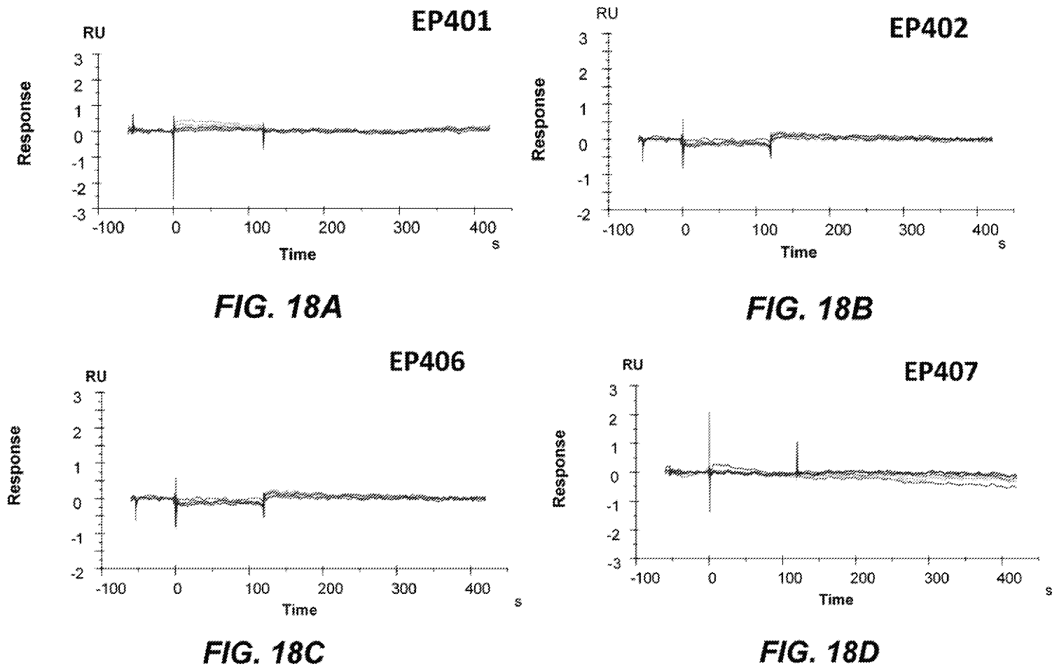

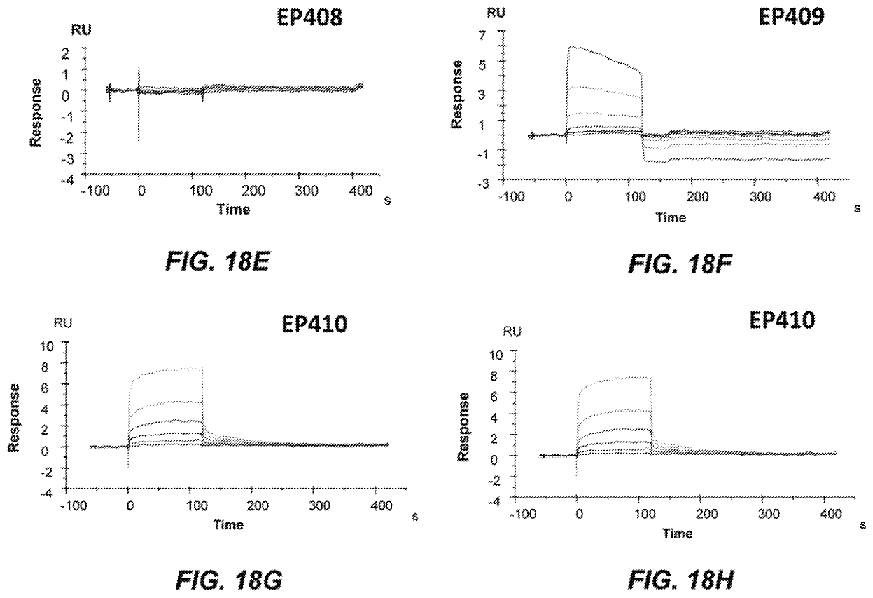

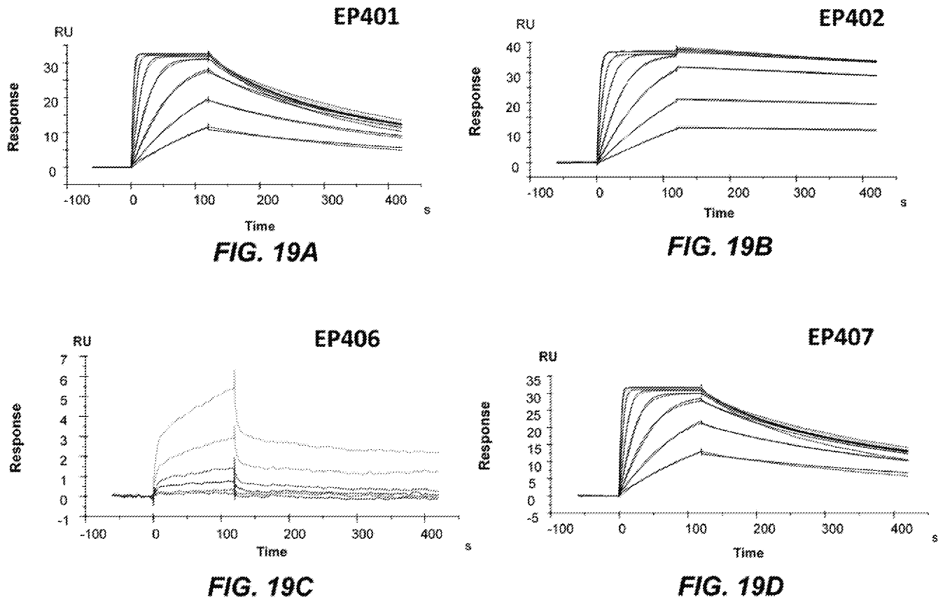

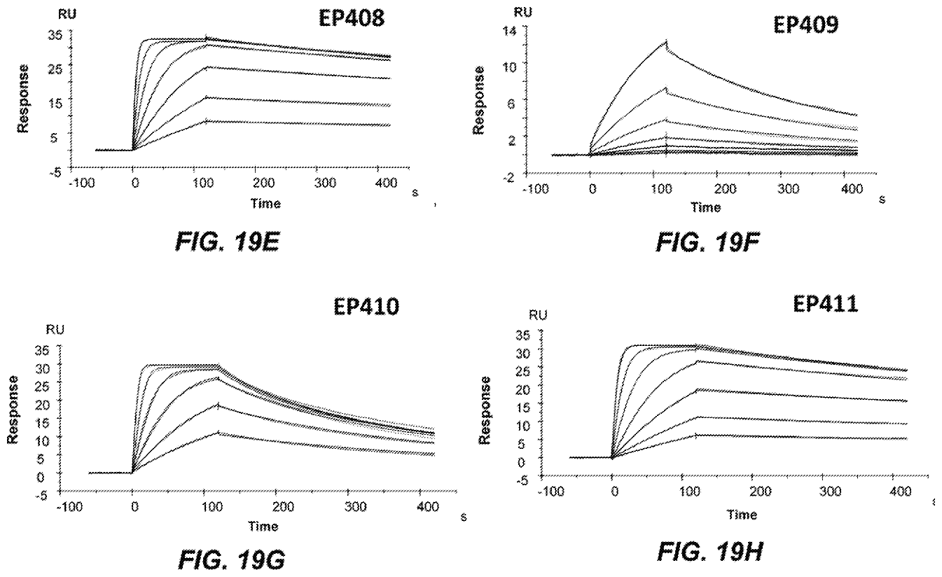

Kinetic analysis of the receptor binding activities of IL2Rβ agonist with IL2Rα reduced activity mutations have been assessed by SPR technology with Biacore T200. The assay was run with Biacore T200 control software version 2.0. For each cycle, 1 ug/mL of human IL2Rβ was captured for 60 seconds at flow rate of 10 ul/min on flow cell 2 in 1×HBSP buffer on Protein A sensor chip. 100 nM of HIS and Flag tag purified each IL2 mutant was 2 fold serial diluted and injected onto both reference flow cell 1 and IL2Rβ captured flow cell 2 for 150 seconds at flow rate of 30 ul/mins followed by wash for 300 seconds. The flow cells were then regenerated with Glycine pH2 for 60 seconds at flow rate of 30 ul/mins. The assay was set up with 8 serial diluted concentration points in 96 well format. The kinetics data was analyzed with Biacore T200 evaluation software 3.0. The specific binding response unit was derived from subtraction of binding to reference flow cell 1 from target flow cell 2. FIGS. 18A-18H show titrated binding of engineered IL2Rβ/a clones to IL2Rα, and FIGS. 19A-19H show titrated binding of engineered IL2Rβ/a clones to IL2Rβ. A description of the clones as well as a summary of the kinetics data are shown in Table 12.

TABLE 12
Summary of binding kinetics of engineered IL2Rβ/IL2Rα clones to human IL2Rα

| | IL2Rα | IL2Rβ | | IL2Rβ | | |
|---|---|---|---|---|---|---|
| | Binding | Binding | IL2Rα | ka | kd | KD |
| Clone | Mutations | Mutations | Binding | (1/Ms) | (1/s) | (M) |
| EP397 | K35G/R38E | WT | NA | NA | NA | NA |
| EP398 | K35G/R38E | EP001 | NA | NA | NA | NA |
| EP399 | K35G/R38E | EP003 | NA | NA | NA | NA |
| EP400 | K35S/F42G | WT | NA | NA | NA | NA |
| EP401 | K35S/F42G | EP001 | ND | 7.21E+06 | 0.006164 | 8.55E−10 |
| EP402 | K35S/F42G | EP003 | ND | 3.68E+06 | 3.97E−04 | 1.08E−10 |
| EP403 | K35L/R38D/F42R | WT | NA | NA | NA | NA |
| EP404 | K35L/R38D/F42R | EP001 | NA | NA | NA | NA |
| EP405 | K35L/R38D/F42R | EP003 | NA | NA | NA | NA |
| EP406 | R38D/Y45S | WT | ND | NA | NA | NA |
| EP407 | R38D/Y45S | EP001 | ND | 8.34E+06 | 0.005094 | 6.11E−10 |
| EP408 | R38D/Y45S | EP003 | ND | 2.56E+06 | 5.98E−04 | 2.33E−10 |
| EP409 | R38V/Y45S | WT | Low | 1.49E+05 | 0.005308 | 3.57E−08 |
| EP410 | R38V/Y45S | EP001 | Low | 7.19E+06 | 0.00594 | 8.27E−10 |
| EP411 | R38V/Y45S | EP003 | Low | 1.82E+06 | 8.39E−04 | 4.61E−10 |

NA: data not available. ND: Non-detectable binding. Low: Binding signal below 10 RU.

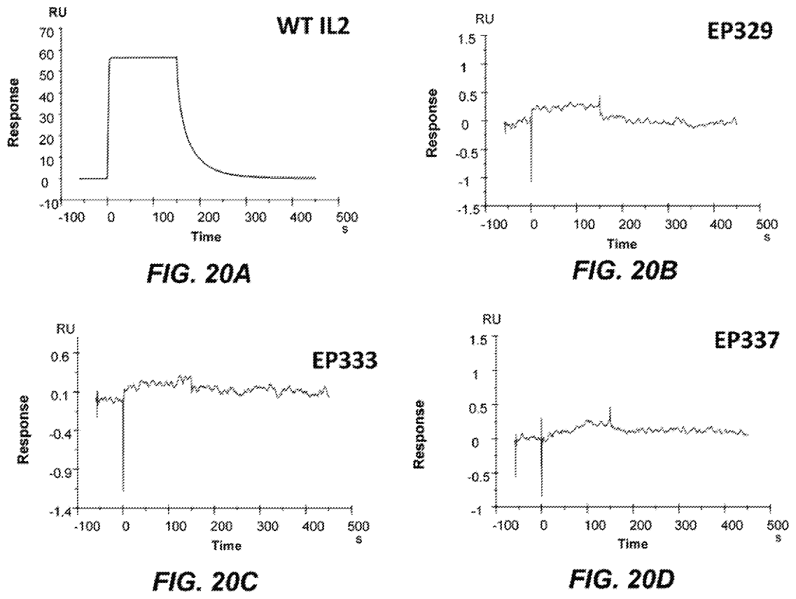

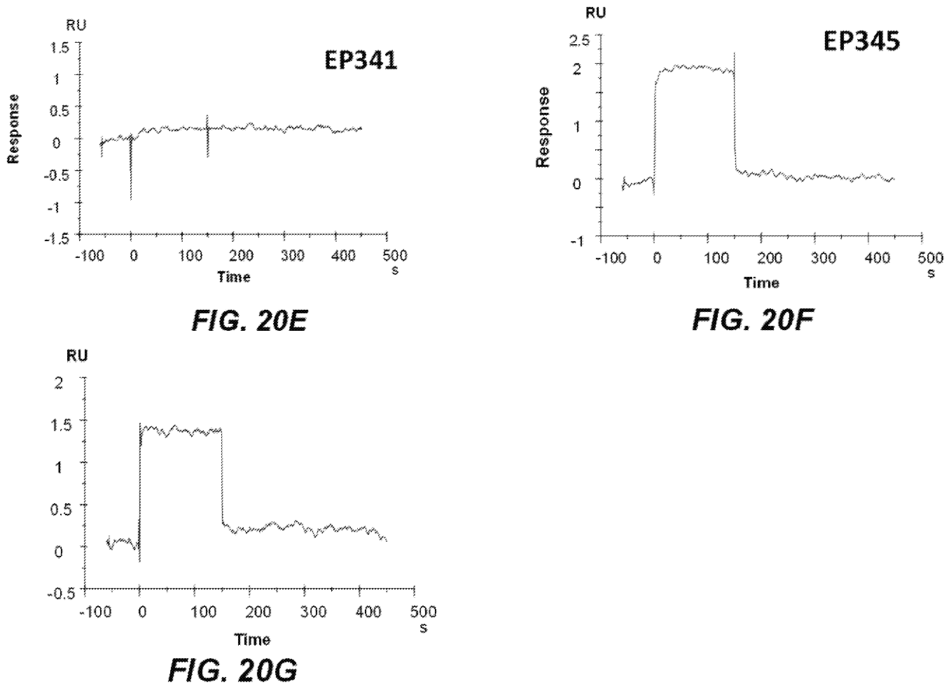

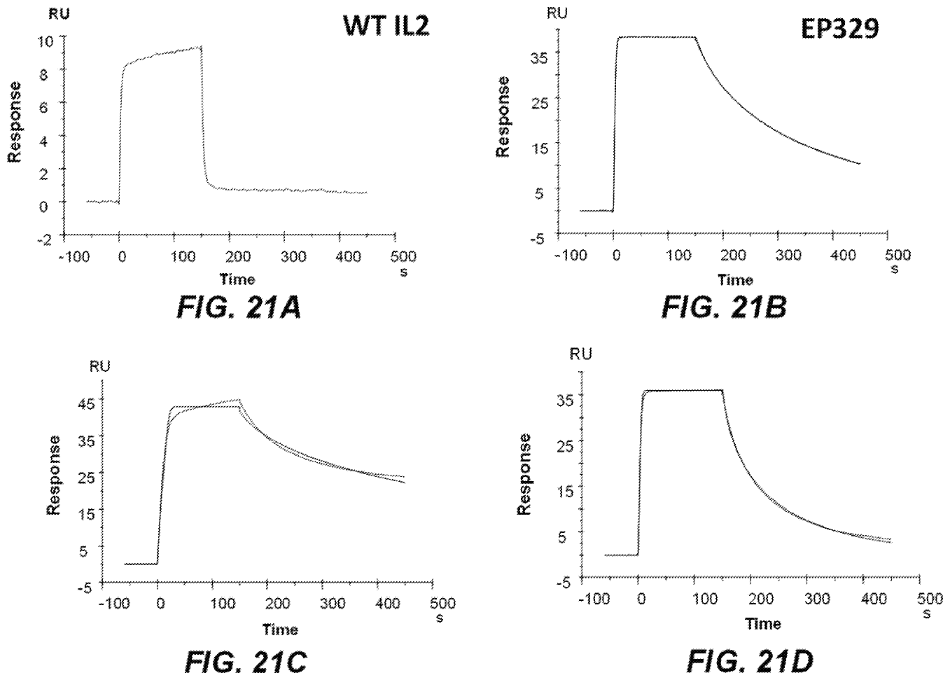

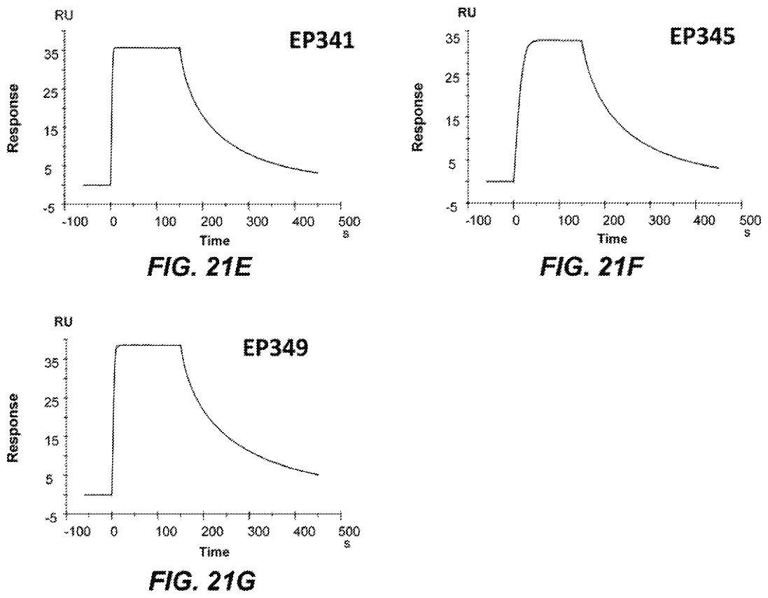

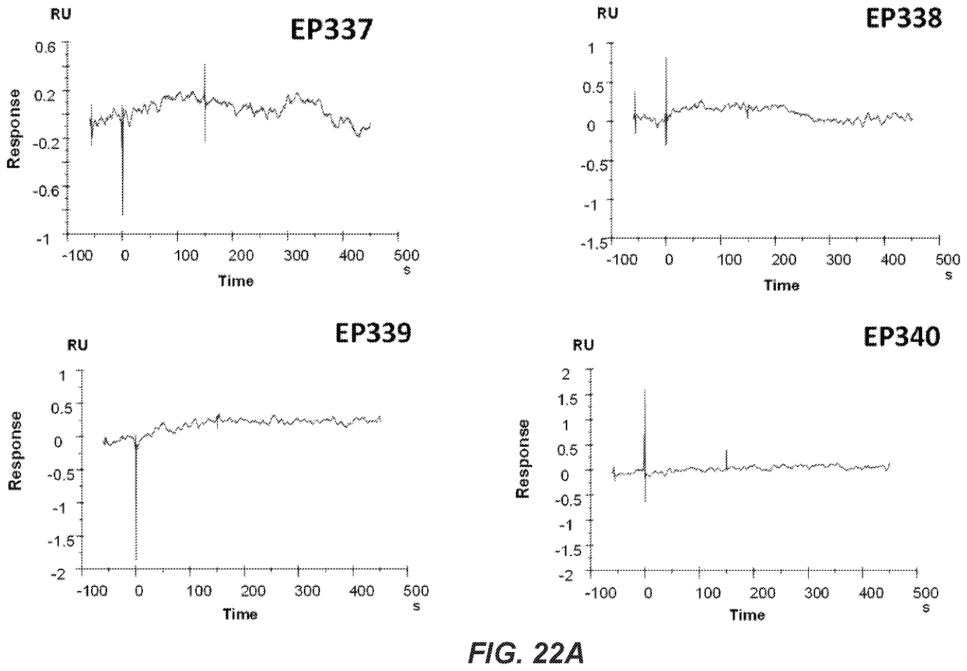

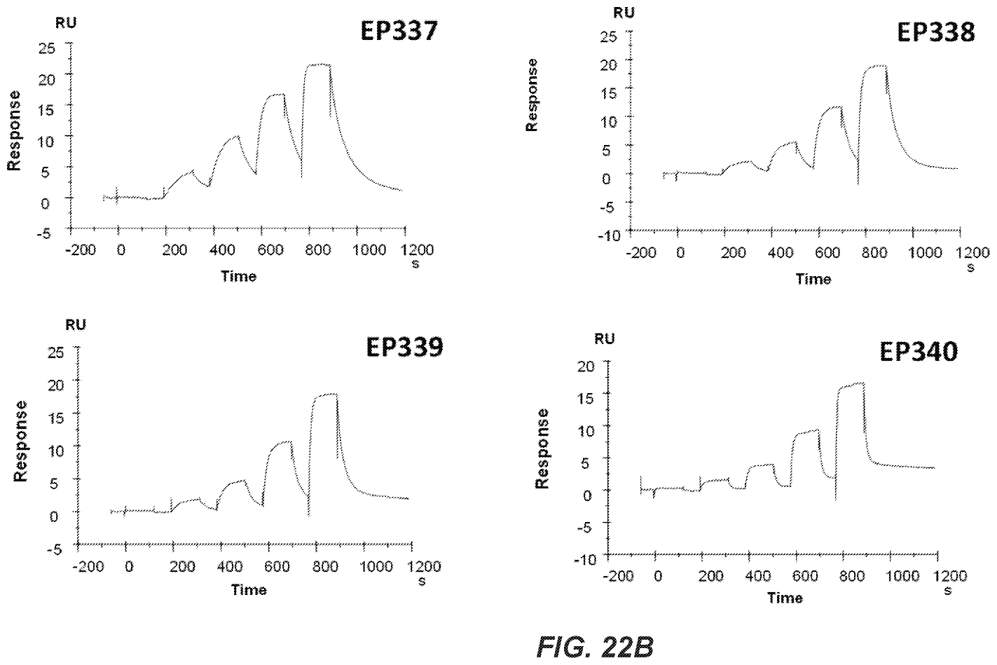

FIGS. 20A-20G show IL2Rα single concentration binding, and FIGS. 21A-21G show IL2Rβ single concentration binding, and FIGS. 22A and 22B show IL2Rα multi-concentration binding, which are summarized in Table 13).

TABLE 13
Summary of binding kinetics of engineered IL2Rβ/IL2Rα clones to human IL2R by SPR

| | Single-Concentration KD (M) | | | | | | | Multi-Concentration KD (M) | | | |
|---|---|---|---|---|---|---|---|---|---|---|---|
| | WT IL2 | EP329 | EP333 | EP337 | EP341 | EP345 | EP349 | EP337 | EP338 | EP339 | EP340 |
| IL2Rα | 1.05E−08 | Very low | Very low | ND | ND | Low | Low | ND | ND | ND | ND |
| IL2Rβ | NA# | 1.22E−09 | 1.41E−09 | 3.80E−09 | 2.54E−09 | 1.25E−08 | 2.79E−09 | 1.44E−08 | 3.83E−08 | 3.59E−08 | NA# |

ND: Non-detectable binding. Low: Binding signal below 5 RU

### Example 14

### ELISA Binding Analysis of Engineered IL2Rα/IL2Rβ Clones

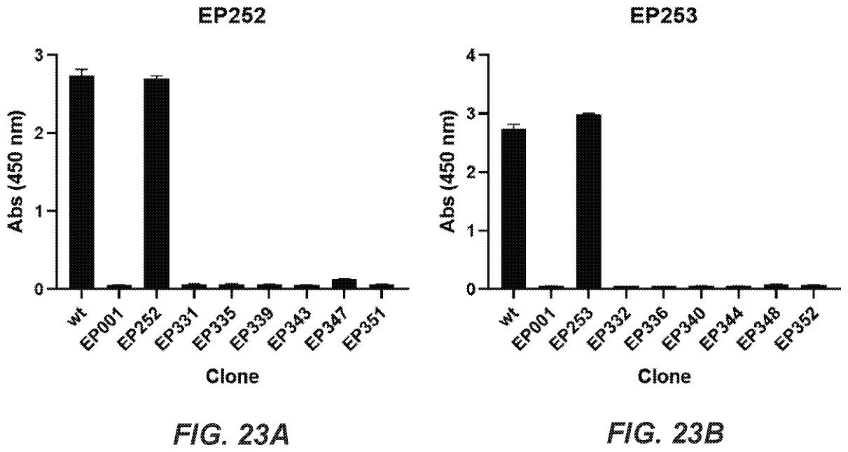

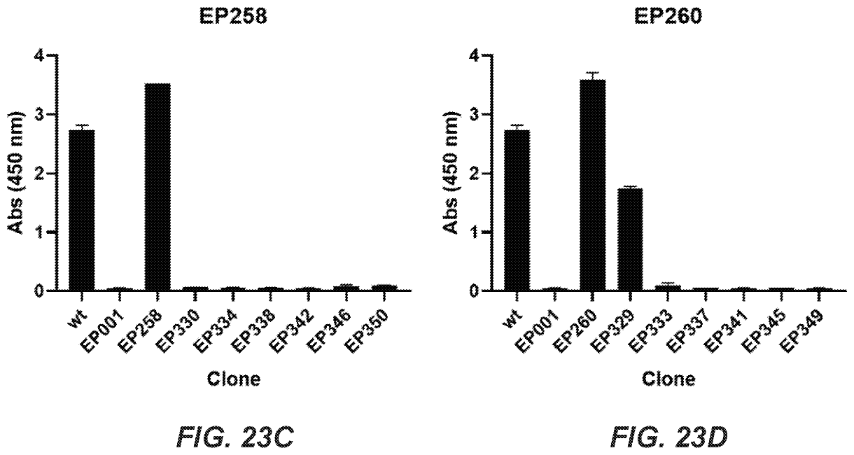

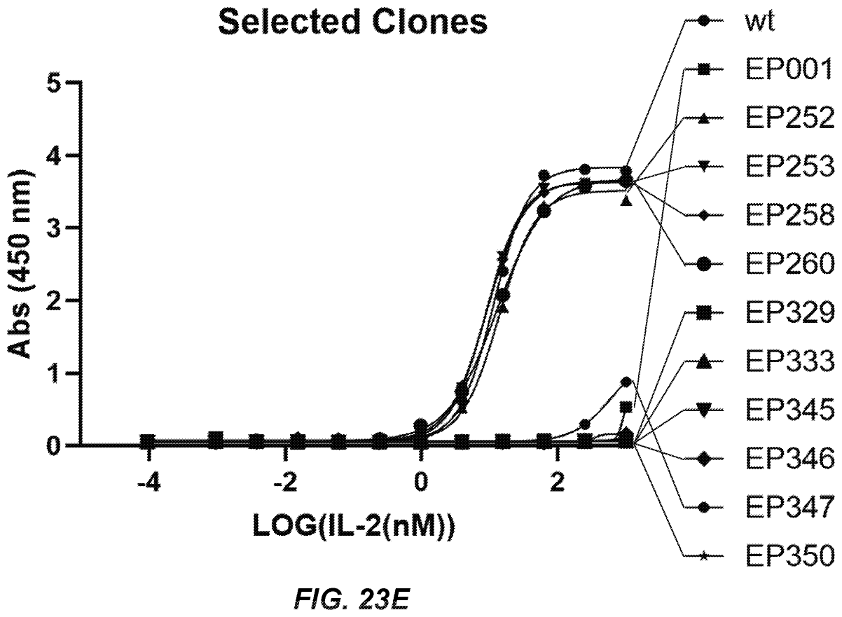

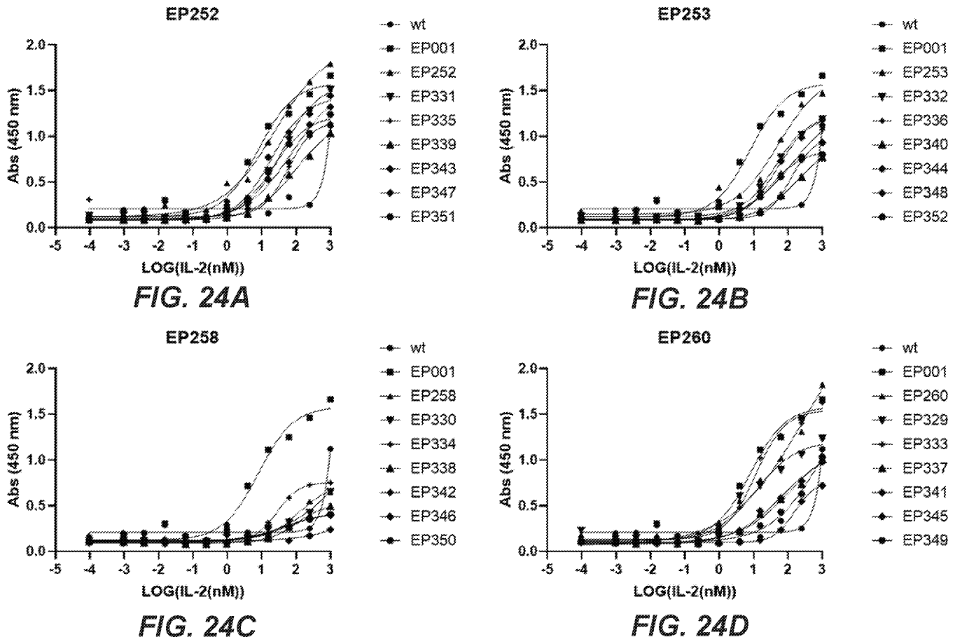

Recombinant Fc-tagged human IL2Rα and IL2Rβ were added in 25 uL of 1×PBS to wells of 384-well plate and incubated overnight at 4° C. to coat the plates. Plates were washed three times with 0.05% Tween20/1×PBS. Plates were blocked with 100 uL of SuperBlock for 1 hr at RT and then washed 3 times with 0.05% Tween20/1×PBS. IL2 mutants were diluted in 0.05% Tween 20/1×PBS from 1000 nM to 0 nM and added to plates for 2 hrs at room temperature. Plates were then washed 6 times with 0.05% Tween20/1×PBS. Anti-HisTag-HRP was diluted 1:5000 in 0.05% Tween20/1×PBS and added to plates for 1 hr at RT. Plates were then washed 6 times with 0.05% Tween 20/1×PBS, and TMB was added to develop blue color. Reactions were stopped with 2N hydrogen sulfide and light absorbance at 450 nm was read on a BioTek plate reader. Single point absorbance for human IL2Rα and titrations for human IL2Rα and IL2Rβ are graphed (FIGS. 23A-23E for IL2Rα, FIGS. 24A-24D for IL2Rβ). A summary of the EC50 values of ELISA binding is shown (Table 14).

TABLE 14
Summary of ELISA binding to human IL2Rα and IL2Rβ

| | EC50 (nM) | | | | | | | | |
|---|---|---|---|---|---|---|---|---|---|
| | WT IL2 | EP001 | EP252 | EP331 | EP335 | EP339 | EP343 | EP347 | EP351 |
| IL2Rα | 11.5 | ND | 13.81 | ND | ND | ND | ND | ND | ND |
| IL2Rβ | ND | 8.1 | 24.5 | 39.3 | 59.1 | 96.3 | 19.7 | 77.6 | 21.2 |
| | | | EP253 | EP332 | EP336 | EP340 | EP344 | EP348 | EP352 |
| IL2Rα | | | 8.9 | ND | ND | ND | ND | ND | ND |
| IL2Rβ | | | 52.8 | 44.0 | 133.0 | 185.0 | 50.7 | 123.7 | 33.8 |
| | | | EP258 | EP330 | EP334 | EP338 | EP342 | EP346 | EP350 |
| IL2Rα | | | 9.1 | ND | ND | ND | ND | ND | ND |
| IL2Rβ | | | 119.2 | ND | 28.6 | 105.3 | 58.9 | ND | 39.9 |
| | | | EP260 | EP329 | EP333 | EP337 | EP341 | EP345 | EP349 |
| IL2Rα | | | 12.9 | ND | ND | ND | ND | ND | ND |
| IL2Rβ | | | 295.9 | 11.4 | 11.4 | 137.9 | 64.1 | 152.1 | 947.8 |

### Example 15

### P-STAT5 Activation of Human PBMCs by Engineered IL2Rα/IL2Rβ Clones

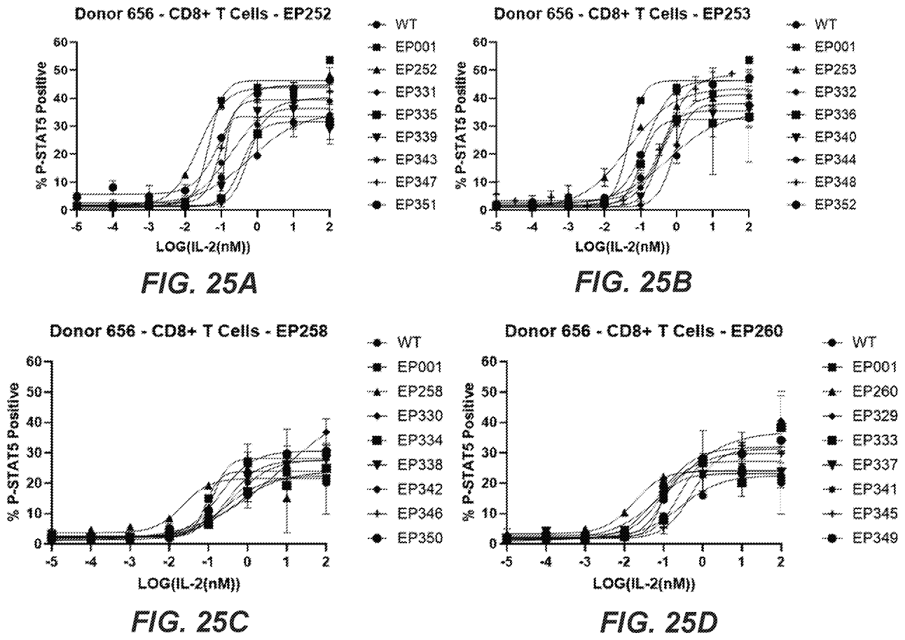

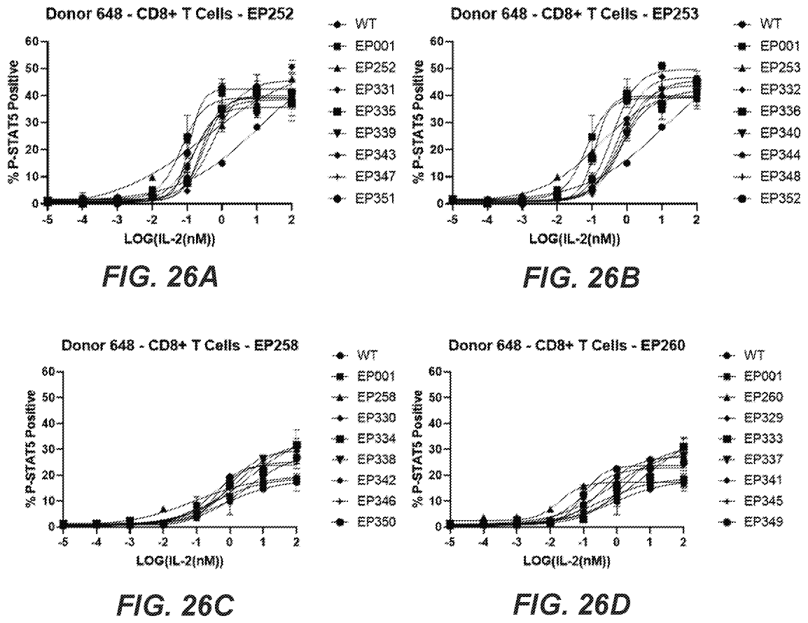

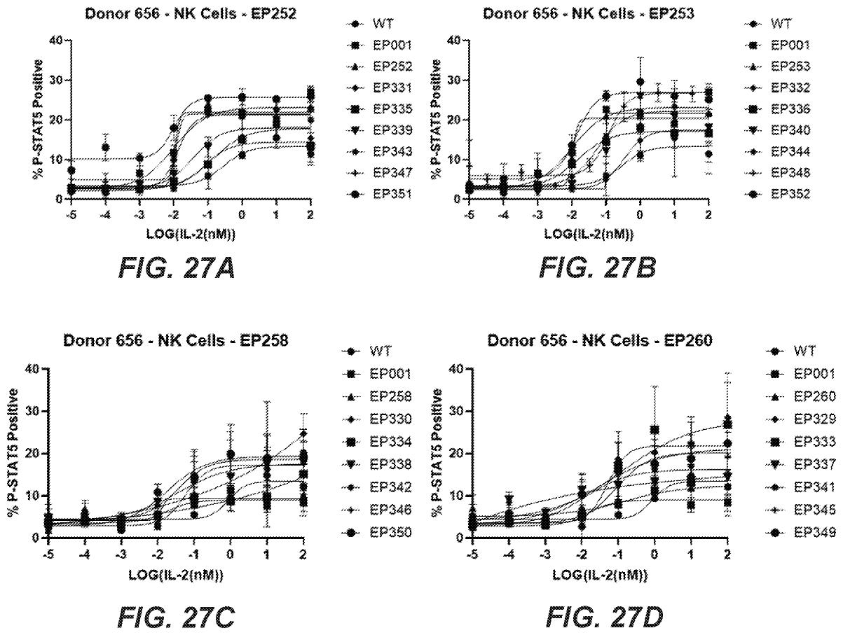

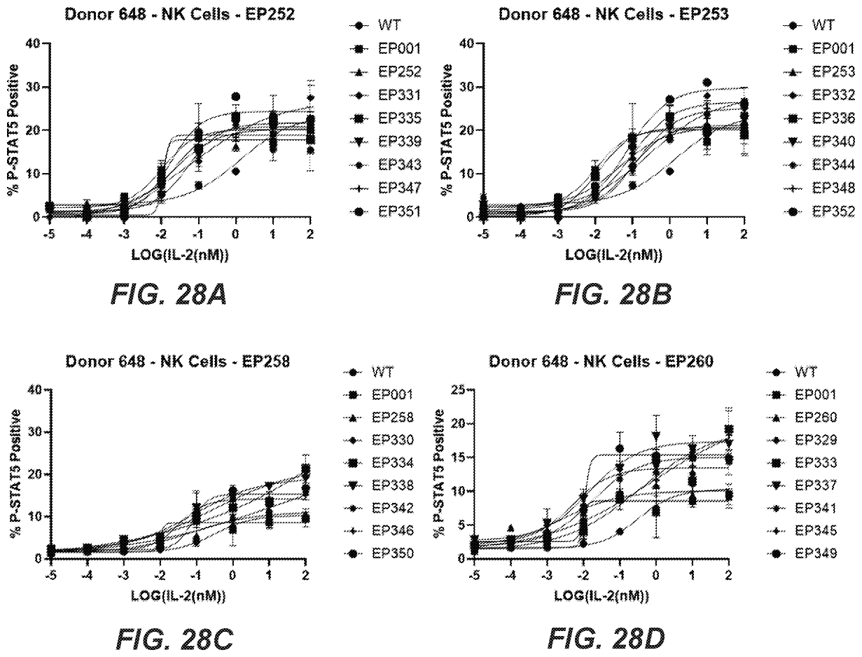

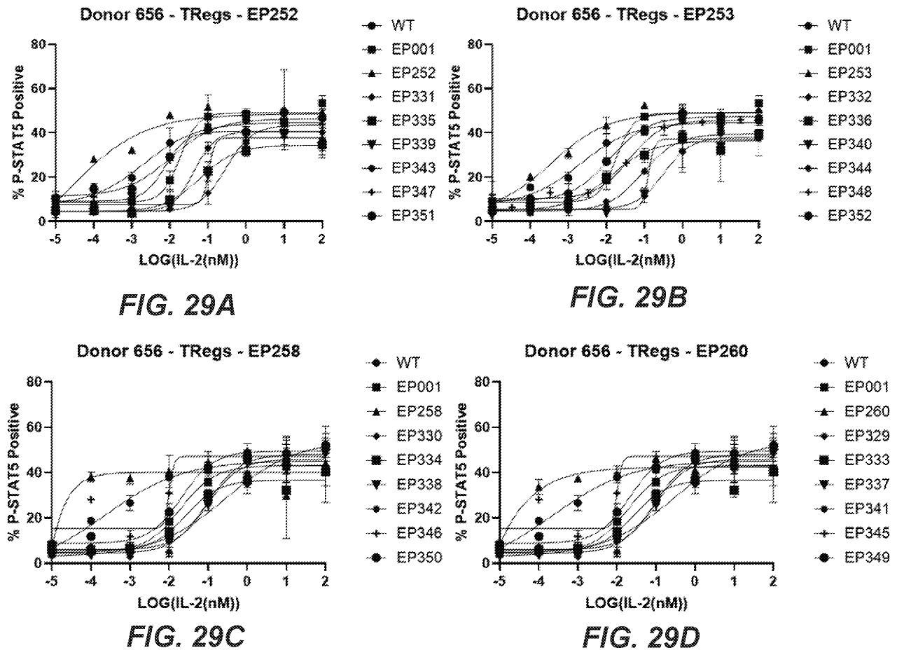

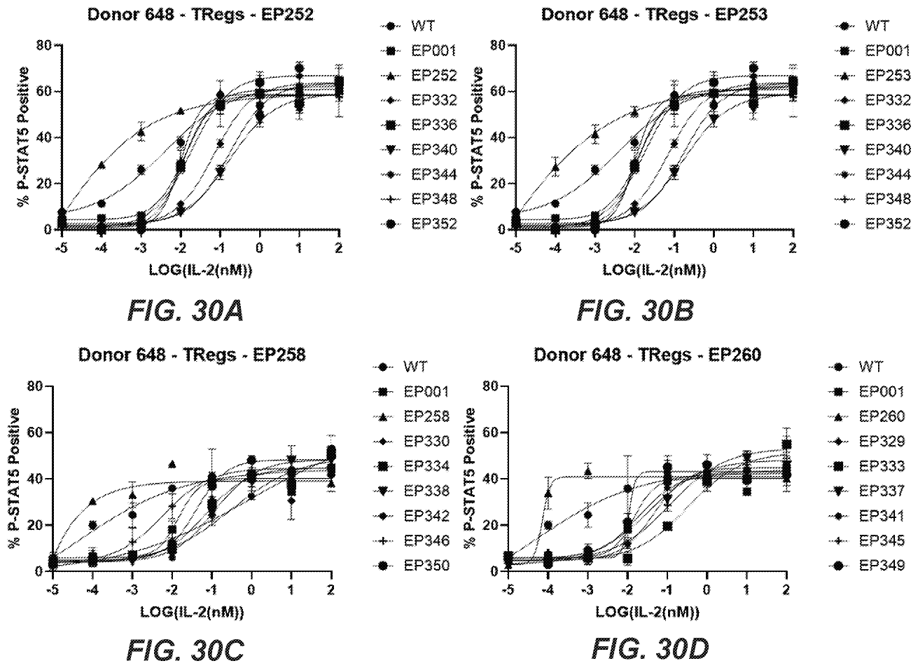

Human PBMCs were isolated from peripheral blood of two donors and plated at 250,000 cells/well in a 96-well plate in 75 μL of media. Cells were rested 1 hr at 37° C. Cells were stimulated with human IL2 WT and engineered His-Flag tagged IL2 at 4× concentration in 25 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIGS. 25A-25D for donor 656 CD8+ T cells, FIGS. 26A-26D for donor 648 CD8=+ T cells, FIGS. 27A-27D for donor 656 NK cells, FIGS. 28A-28D for donor 648 NK cells, FIGS. 29A-29D for donor 656 T regulatory cells, and FIGS. 30A-30D for donor 648 T regulatory cells). A summary of the EC50 values of P-STAT5 activation in each cell type is shown in Table 15 for blood donor 1 and Table 16 for blood donor 2.

TABLE 15
Summary of P-STAT5 activation of human CD8+ T cells, NK cells and Trees for donor 1.

| | EC50 (nM) | | | | | | | | |
|---|---|---|---|---|---|---|---|---|---|
| | Blood Donor 1 | | | | | | | | |
| | WT | EP001 | EP252 | EP331 | EP335 | EP339 | EP343 | EP347 | EP351 |
| CD8 + T Cells | 6.1E−01 | 4.7E−02 | 2.4E−02 | 5.6E−01 | 3.6E−01 | 1.2E−01 | 1.9E−01 | 9.4E−02 | 9.7E−02 |
| NK Cells | 2.8E−01 | 1.0E−02 | 1.1E−02 | 1.3E−01 | 8.2E−02 | 3.8E−02 | 1.3E−02 | 1.2E−02 | 9.9E−03 |
| T Regulatory | 2.0E−03 | 1.8E−02 | 2.5E−05 | 2.9E−01 | 6.1E−02 | 1.0E−01 | 4.3E−02 | 7.6E−03 | 1.1E−02 |
| Cells | | | | | | | | | |
| | WT | EP001 | EP253 | EP332 | EP336 | EP340 | EP344 | EP348 | EP352 |
| CD8 + T Cells | 6.1E−01 | 4.7E−02 | 4.5E−02 | 8.1E−01 | 1.0E−01 | 3.1E−01 | 4.0E−01 | 4.8E−01 | 1.4E−01 |
| NK Cells | 2.8E−01 | 1.0E−02 | 1.1E−02 | 3.2E−01 | 1.5E−02 | 1.0E−01 | 5.5E−02 | 1.2E−01 | 1.3E−02 |
| T Regulatory | 2.0E−03 | 1.8E−02 | 3.7E−04 | 3.2E−01 | 1.3E−02 | 1.3E−01 | 9.1E−02 | 3.9E−02 | 1.2E−02 |
| Cells | | | | | | | | | |
| | WT | EP001 | EP258 | EP330 | EP334 | EP338 | EP342 | EP346 | EP350 |
| CD8 + T Cells | 2.7E−01 | 8.6E−02 | 2.2E−02 | 8.0E+00 | 4.7E−01 | 3.3E−01 | 4.0E−01 | 1.3E−01 | 2.0E−01 |
| NK Cells | 8.7E−01 | 1.7E−02 | 1.3E−02 | ND | ND | 3.8E−02 | 5.7E−02 | 3.4E−02 | 1.8E−02 |
| T Regulatory | 2.4E−04 | 1.9E−02 | <1E−4 | 2.8E−01 | 2.7E−02 | 9.6E−02 | 9.1E−02 | N.D. | 2.1E−02 |
| Cells | | | | | | | | | |
| | WT | EP001 | EP260 | EP329 | EP333 | EP337 | EP341 | EP345 | EP349 |
| CD8 + T Cells | 2.7E−01 | 8.6E−02 | 1.7E−02 | 1.6E−01 | 2.5E−01 | 6.3E−02 | 1.0E−01 | 5.2E−01 | 8.4E−02 |
| NK Cells | 8.7E−01 | 1.7E−02 | 1.5E−01 | 1.4E−01 | 5.0E−02 | ND | 1.2E−02 | 8.1E−02 | 2.8E−02 |
| T Regulatory | 2.4E−04 | 1.9E−02 | <1E−4 | 2.8E−01 | 2.7E−02 | 9.6E−02 | 9.1E−02 | ND | 2.1E−02 |
| Cells | | | | | | | | | |

TABLE 16
Summary of P-STAT5 activation of human CD8 + T cells, NK cells and Tregs for donor 2.

| | EC50 (nM) | | | | | | | | |
|---|---|---|---|---|---|---|---|---|---|
| | Blood Donor 2 | | | | | | | | |
| | WT | EP001 | EP252 | EP331 | EP335 | EP339 | EP343 | EP347 | EP351 |
| CD8 + T Cells | 9.9E+00 | 6.8E−02 | 3.5E−01 | 5.7E−01 | 2.6E−01 | 3.0E−01 | 1.5E−01 | 1.7E−01 | 1.1E−01 |
| NK Cells | 1.3E+00 | 1.0E−02 | 1.2E−02 | 7.6E−02 | 2.1E−02 | 5.0E−02 | 1.1E−02 | 2.1E−02 | 1.8E−02 |
| T Regulatory | 3.8E−03 | 1.3E−02 | 2.5E−06 | 7.0E−02 | 1.1E−02 | 1.7E−01 | 1.7E−01 | 1.0E−02 | 1.8E−02 |
| Cells | | | | | | | | | |
| | WT | EP001 | EP253 | EP332 | EP336 | EP340 | EP344 | EP348 | EP352 |
| CD8 + T Cells | 9.9E+00 | 6.8E−02 | 1.6E−01 | 5.6E−01 | 1.2E−01 | 6.5E−01 | 8.0E−01 | 6.2E−01 | 3.8E−01 |
| NK Cells | 1.3E+00 | 1.0E−02 | 4.5E−02 | 8.6E−02 | 1.2E−02 | 9.2E−02 | 2.9E−01 | 1.3E−01 | 7.1E−02 |
| T Regulatory | 3.8E−03 | 1.3E−02 | 1.8E−06 | 7.0E−02 | 1.1E−02 | 1.7E−01 | 1.7E−01 | 1.0E−02 | 1.8E−02 |
| Cells | | | | | | | | | |
| | WT | EP001 | EP258 | EP330 | EP334 | EP338 | EP342 | EP346 | EP350 |
| CD8 + T Cells | 7.5E−01 | 3.1E−01 | 8.8E−02 | 1.6E+00 | 3.4E+00 | 9.1E−01 | 2.9E+00 | 3.2E−01 | 3.9E−01 |
| NK Cells | 3.2E−01 | 1.0E−02 | 7.6E−02 | 2.9E−01 | ND | 8.7E−02 | 1.2E+01 | 3.2E−02 | 7.6E−02 |
| T Regulatory | 4.5E−05 | 1.4E−02 | <1E−4 | 9.1E−01 | 6.6E−02 | 1.2E−01 | 3.0E−01 | 5.8E−03 | 4.0E−02 |
| Cells | | | | | | | | | |
| | WT | EP001 | EP260 | EP329 | EP333 | EP337 | EP341 | EP345 | EP349 |
| CD8 + T Cells | 7.5E−01 | 3.1E−01 | 1.9E−02 | 9.6E−02 | 7.7E+00 | 6.6E−01 | 3.4E−01 | 1.6E+00 | 9.7E−02 |
| NK Cells | 3.2E−01 | 1.0E−02 | 2.7E−03 | 4.8E−03 | 7.5E−01 | 1.5E−02 | 1.7E−02 | 3.6E−01 | 1.1E−02 |
| T Regulatory | 4.5E−05 | 1.4E−02 | <1E−4 | 2.0E−02 | 3.4E−01 | 3.2E−02 | 4.5E−02 | 1.2E−01 | ND |
| Cells | | | | | | | | | |

### Example 16

### P-STAT5 Activation of Murine Cells by Engineered IL2Rα/IL2Rβ Clones

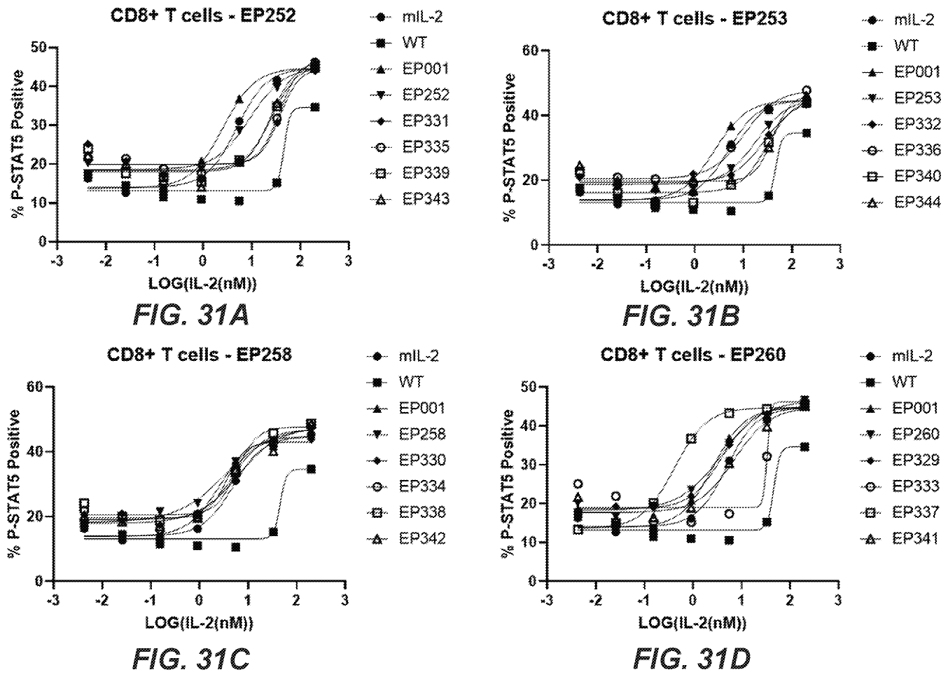

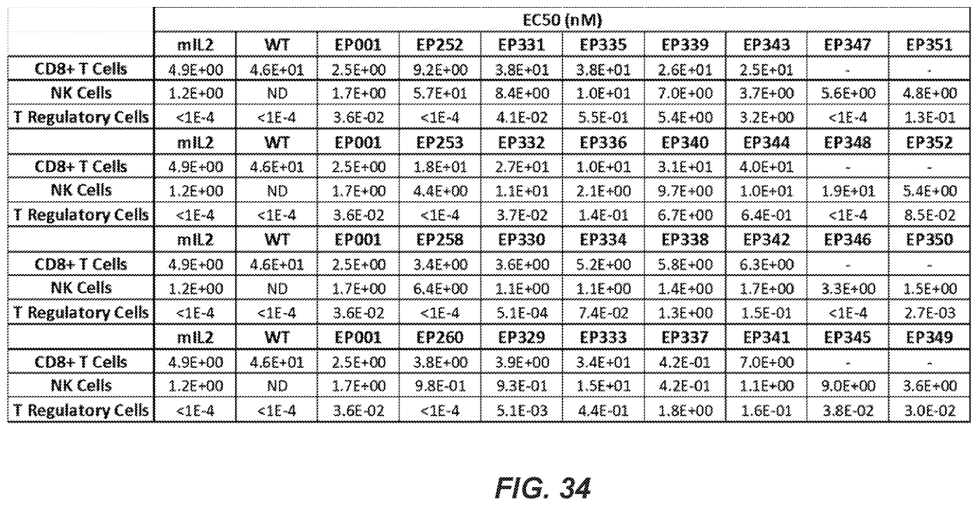

Murine splenocytes were plated at 250,000 cells/well in a 96-well plate in 75 μL of media. Cells were rested 1 hr at 37° C. Cells were stimulated with human IL2 WT and engineered His-Flag tagged IL2 at 4× concentration in 25 μL for 20 min at 37° C. Stimulated mouse splenocytes were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. The % of cells that were p-STAT5+ was determined and graphed versus each IL2 titration (FIGS. 31A-31D). A summary of the EC50 values of P-STAT5 activation in each cell type is shown in FIG. 34.

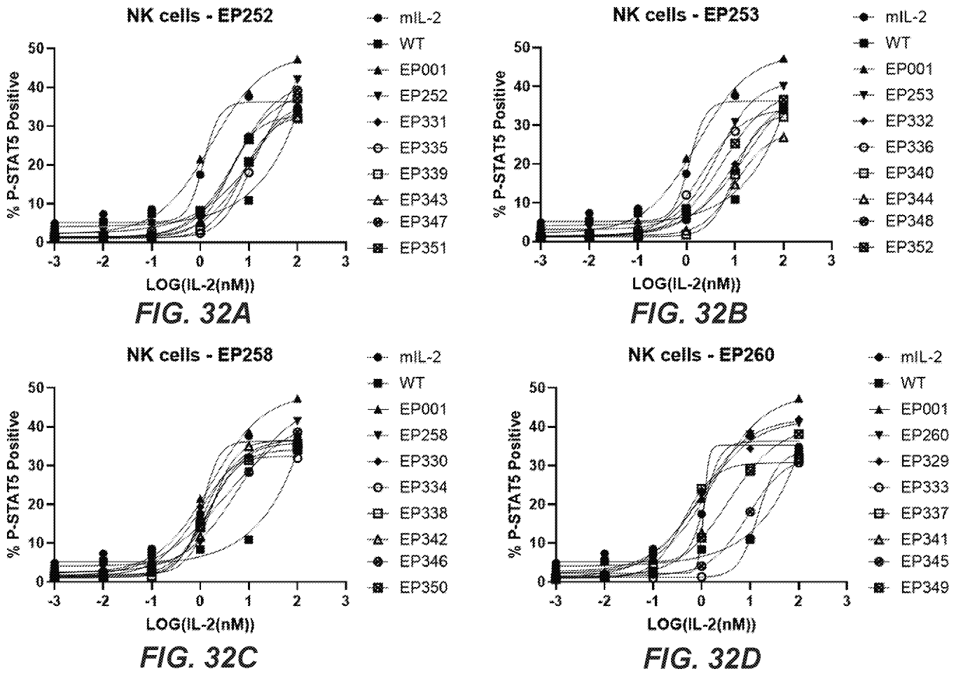

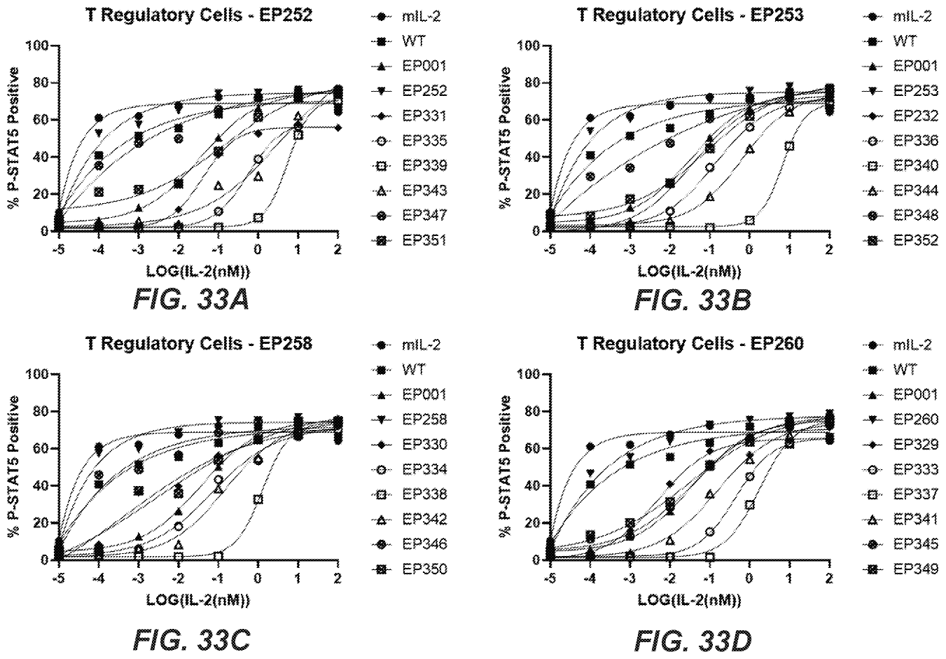

Isolated NK cells or mouse T regulatory cells were plated at 20,000 cells/well in a 96-well plate in 75 μL of media. Cells were rested 1 hr at 37° C. Cells were stimulated with human IL2 WT and engineered His-Flag tagged IL2 at 4× concentration in 25 μL for 20 min at 37° C. Stimulated mouse NK cells or mouse T regulatory cells were immediately fixed, permeabilized, stained for P-STAT5 and visualized on the Attune flow cytometer. The % of cells that were p-STAT5+ was determined and graphed versus each IL2 titration (FIGS. 32A-32D, 33A-33D).

A summary of the EC50 values of P-STAT5 activation in each cell type is shown in FIG. 34.

### Example 17

### Design of IL2Rβ Agonist Fc-Fusion Proteins

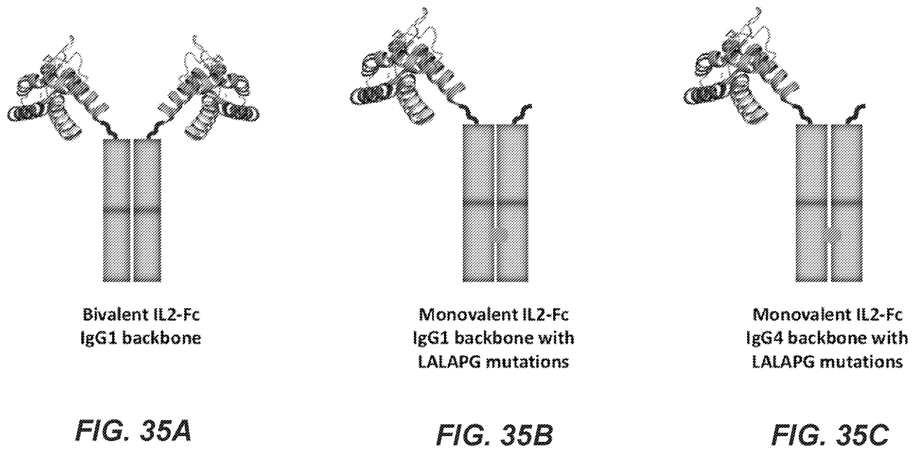

To generate bivalent IL2Rβ agonist Fc-fusion protein, the protein sequences encoding engineered IL2 polypeptides of EP003 (SEQ ID NO: 2), EP007 (SEQ ID NO: 4), EP002 (SEQ ID NO: 6), EP004 (SEQ ID NO: 09), EP001 (SEQ ID NO: 11), EP006 (SEQ ID NO: 16), EP009 (SEQ ID NO: 18), and EP005 (SEQ ID NO: 19) were fused to the N-terminal site of the constant frame sequence of human IgG1 isoform to produce engineered agonist-Fc fusion proteins (SEQ ID NOs: 44, 46, 48, 51, 53, 58, and 61). L234A, L235A and P329G mutations in the human IgG1 were introduced to eliminate complement binding and Fc-γ dependent antibody-dependent cell-mediated cytotoxity (ADCC) effects (Lo et al., JBC 2017) (FIG. 35A).

To generate monovalent IL2Rβ agonist Fc-fusion protein, the protein sequences encoding engineered IL2 polypeptides of EP003 (SEQ ID NO: 2), EP007 (SEQ ID NO: 4), EP002 (SEQ ID NO: 6), EP004 (SEQ ID NO: 09), EP001 (SEQ ID NO: 11), EP006 (SEQ ID NO: 16), EP009 (SEQ ID NO: 18), and EP005 (SEQ ID NO: 19) were fused to the N-terminal site of the constant frame sequences of respective human IgG1 and IgG4 isoforms, to produce engineered agonist-Fc fusion proteins (SEQ ID NOs: 44, 46, 48, 51, 53, 58, and 61). The knob mutations of S354C, T366W and K409A were introduced to the constructs. The hole mutations of Y349C, T366S, L368A, F405K, Y407V were introduced to CH2 and CH3 fragments of IgG1 and IgG4, respectively. The L234A, L235A and P329G mutations in the human IgG1 were introduced to eliminate complement binding and Fc-γ dependent antibody-dependent cell-mediated cytotoxicity (ADCC) effects (Lo et al., JBC 2017) (FIGS. 35B & 35C). The DNA encoding the entire Fc fusion agonist protein was then synthesized with codon optimized for mammalian cell expression, and subcloned to pCDNA3.4 (Invitrogen).

### Example 18

### Production of IL2Rβ Agonist Fc Fusion Proteins

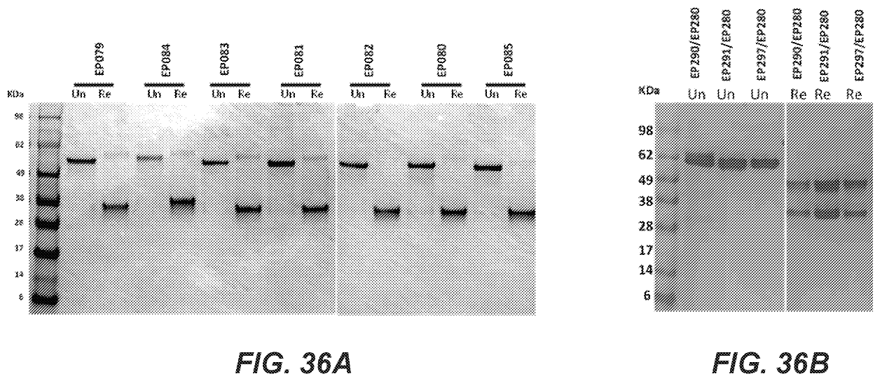

For bivalent IL2-Fc fusion protein production, the agonist was expressed transiently in ExpiHEK293-F cells in free style system (Invitrogen) according to standard protocol. The cells were grown in above conditions for seven days before harvesting. The supernatant was collected by centrifugation and filtered through a 0.2 μm PES membrane. The Fc fusion agonist first was purified by Mab Select PrismA protein A resin (GE Health). The protein was eluted with 100 mM Gly pH2.5+150 mM NaCl and quickly neutralized with 20 mM citrate pH 5.0+300 mM NaCl. The agonist protein was then concentrated to 1 mL and further purified by a Superdex 200 Increase 10/300 GL column. The monomeric peak fractions were pooled and concentrated. The final purified protein has endotoxin of lower than 10EU/mg and kept in 20 mM citrate pH 5.0+300 mM NaCl. The purified IL2-Fc fusion agonists were run on an SDS gel (4-12% Bis-Tris Bolt gel, with MES running buffer), comparing samples of each treated under reducing versus non-reducing conditions (FIG. 36A).

For monovalent IL2-Fc fusion protein production, the “knob” and “hole” constructs in respective IgG1 and IgG4 backbone format were transfected to ExpiHEK293-F cells with the ratio of 1:1. The cells were grown in above conditions for five days before harvesting. The supernatant was collected by centrifugation and filtered through a 0.2 μm PES membrane. The Fc fusion agonist first was purified by MabSelect PrismA protein A resin (GE Health). The protein was eluted with 100 mM Gly pH2.5+150 mM NaCl and quickly neutralized with 20 mM citrate pH 5.0+300 mM NaCl. The agonist protein was then concentrated to 1 mL and further purified by a Superdex 200 Increase 10/300 GL column. The monomeric peak fractions were pooled and concentrated. The final purified protein has endotoxin of lower than 10EU/mg and kept in 20 mM citrate pH 5.0+300 mM NaCl. The purified monovalent IL2-Fc fusion agonists were run on an SDS gel (4-12% Bis-Tris Bolt gel, with MES running buffer), comparing samples of each treated under reducing versus non-reducing conditions (FIG. 36B).

### Example 19

### ELISA Binding Analysis of IL2Rβ Agonist Fc-Fusion Proteins

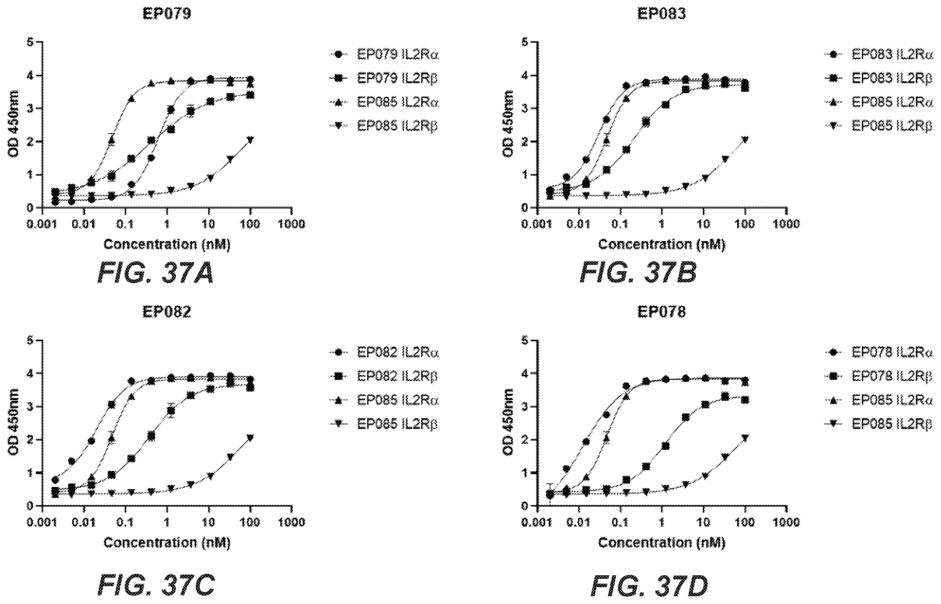

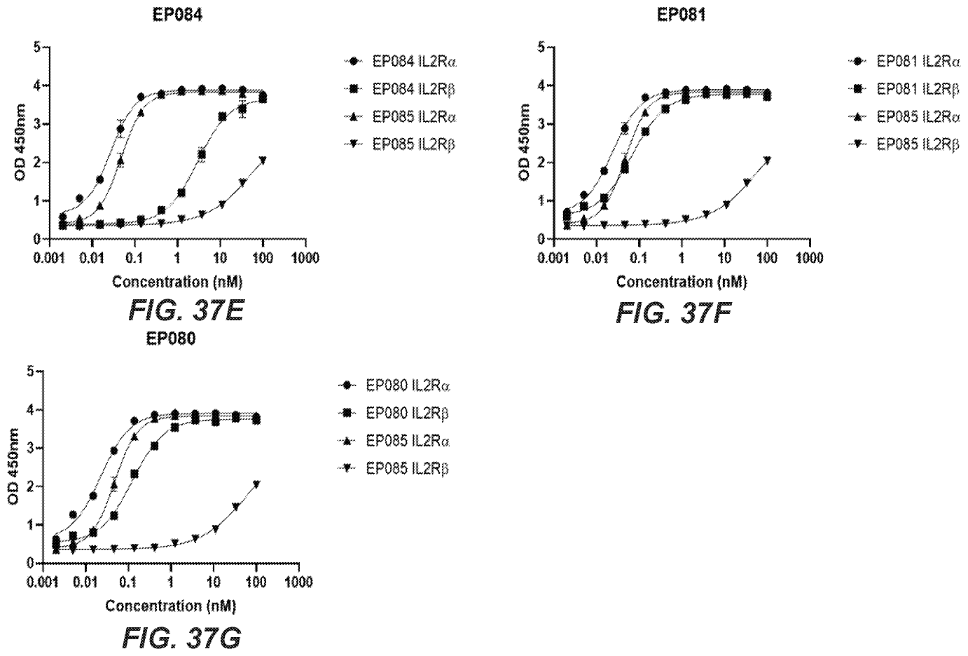

For bivalent Fc-fusion proteins, human IL2Rα, and human IL2Rβ were each immobilized in a 384 well plate at final concentration of 2 μg/mL in 1×PBS in total volume of 25 μL per well. The plate was incubated overnight at 4° C. followed by blocking with 80 μL of superblock per well for 1 hour. The purified engineered IL2 mutant Fc fusion protein at 100 nM was 3-fold serial diluted 12 times. Each dilution was added to IL2Rα and IL2Rβ wells in parallel. The engineered IL2 mutant binding was detected by adding 50 μL of anti-human Fc HRP diluted at 1:5000 in 1×PBST. In between each step, the plate was washed 3 times with 1×PBST in a plate washer. The plate was then developed with 25 μl of TMB substrate for 5 mins and stopped by adding 25 μl of 2N sulfuric acid. The plate was read at OD450 nm Biotek plate reader and the EC50 was analyzed with Prism 8.1 software. Absorbance versus IL-2 concentration is graphed for human IL2Rα and IL2Rβ (FIGS. 37A-37G). A summary of the EC50 values of ELISA binding is shown (Table 17).

TABLE 17
Summary of receptor binding analysis of
bivalent IL2Rβ Fc fusion proteins

| | | | Relative | |
|---|---|---|---|---|
| | | | activity | |
| | IL2α | IL2β | IL2Rα | IL2Rβ |
| | EC50 | EC50 | (EC50: | (EC50: |
| Clones | (nM) | (nM) | fold) | fold) |
| EP085 | 5.0E−02 | 3.0E−02* | 1 | 1 |
| EP079 | 6.4E−01 | 4.1E−01 | −12.7 | 727 |
| EP083 | 4.6E−02 | 2.8E−01 | 1.09 | 1079 |
| EP082 | 2.8E−02 | 2.5E−01 | 1.78 | 1189 |
| EP078 | 4.1E−02 | 2.1E+00 | 1.24 | 142 |
| EP084 | 4.1E−02 | 2.6E+00 | 1.23 | 117 |
| EP081 | 8.7E−02 | 2.0E−01 | −1.73 | 1528 |
| EP080 | 5.6E−02 | 2.3E−01 | −1.17 | 1278 |

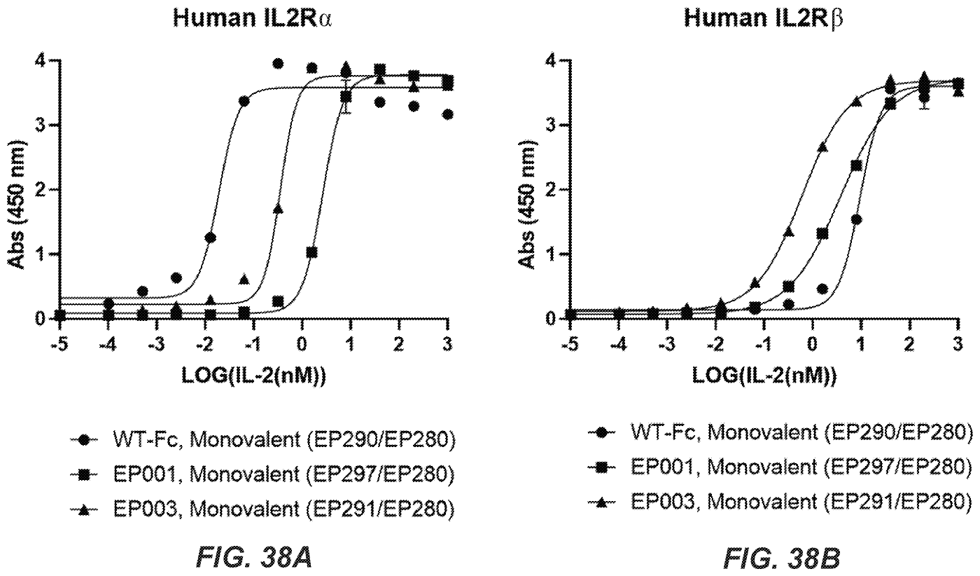

For monovalent Fc-fusion proteins, recombinant His-tagged human IL2Rα and IL2Rβ were added in 25 uL of 1×PBS to wells of 384-well plate and incubated overnight at 4° C. to coat the plates. Plates were washed three times with 0.05% Tween20/1×PBS. Plates were blocked with 100 uL of SuperBlock for 1 hr at RT and then washed 3 times with 0.05% Tween20/1×PBS. IL2 mutants were diluted in 0.05% Tween 20/1×PBS from 1000 nM to 0 nM and added to plates for 2 hrs at room temperature. Plates were then washed 6 times with 0.05% Tween20/1×PBS. Anti-HisTag-HRP was diluted 1:5000 in 0.05% Tween20/1×PBS and added to plates for 1 hr at RT. Plates were then washed 6 times with 0.05% Tween20/1×PBS, and TMB was added to develop blue color. Reactions were stopped with 2N hydrogen sulfide and light absorbance at 450 nm was read on a BioTek plate reader. Absorbance versus IL2 concentration is graphed for human IL2Rα and IL2Rβ (FIGS. 38A-38B). A summary of the EC50 values of ELISA binding is shown (Table 18).

TABLE 18
Summary of receptor binding analysis
of monovalent IL2Rβ Fc fusion proteins

| | EC50 (nM) | |
|---|---|---|
| | hIL2Rα | hIL2Rβ |
| WT-Fc, Monovalent (EP290/EP280) | 1.9E−02 | 9.4E+00 |
| EP001, Monovalent (EP297/EP280) | 2.7E+00 | 3.8E+00 |
| EP003, Monovalent (EP291/EP280) | 3.6E−01 | 6.0E−01 |

### Example 20

### Binding Kinetics of Monovalent IL2Rβ Fc Fusion Proteins

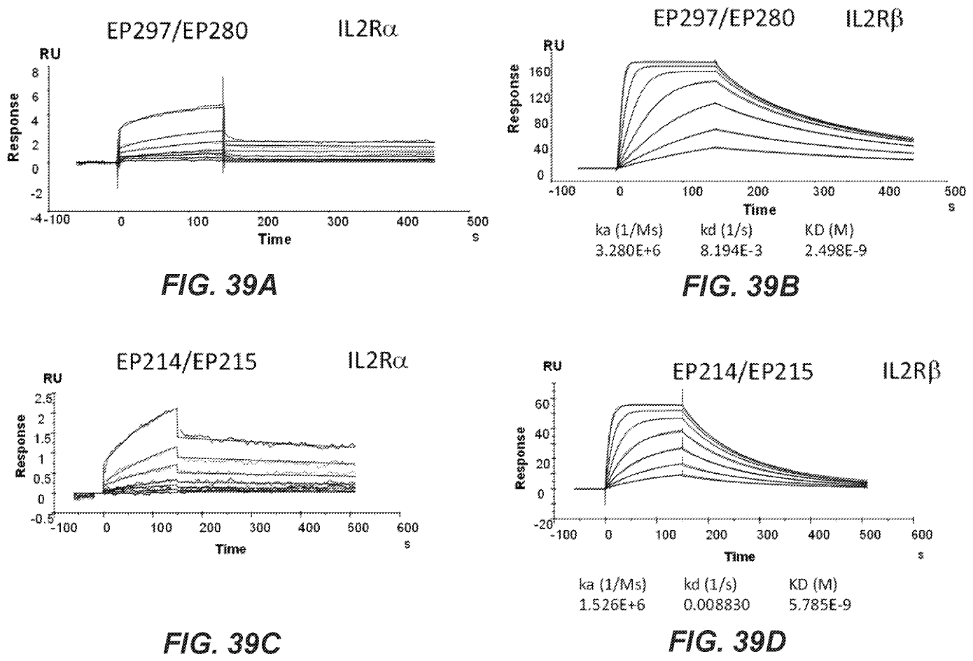

Binding kinetics of monovalent IL2Rβ Fc fusion proteins have been analyzed by SPR technology with Biacore T200. Briefly, anti-hFc antibody was immobilized on flow cell 1 and 2. For each cycle, 1 ug/mL of IL2 Fc fusion protein was captured for 60 seconds at flow rate of 10 ul/min on flow cell 2 in 1×HBSP buffer on anti-hFc immobilized chip. 100 nM IL2Rα-HIS tagged or IL2Rβ-HIS tagged was 2-fold serial diluted and injected onto both reference flow cell 1 and IL2 Fc fusion protein were captured at flow cell 2 for 150 seconds at flow rate of 30 ul/mins. 300 seconds wash was applied after the last injection. The assay was set up with 8 serial diluted concentration points in 96 well format. The kinetics data was analyzed with Biacore T200 evaluation software 3.0. The specific binding response unit was derived from subtraction of binding to reference flow cell 1 from target flow cell 2 (FIGS. 39A-39D).

### Example 21

### P-STAT5 Activation of Human PBMCs by IL2Rβ Agonist Fc-Fusion Proteins

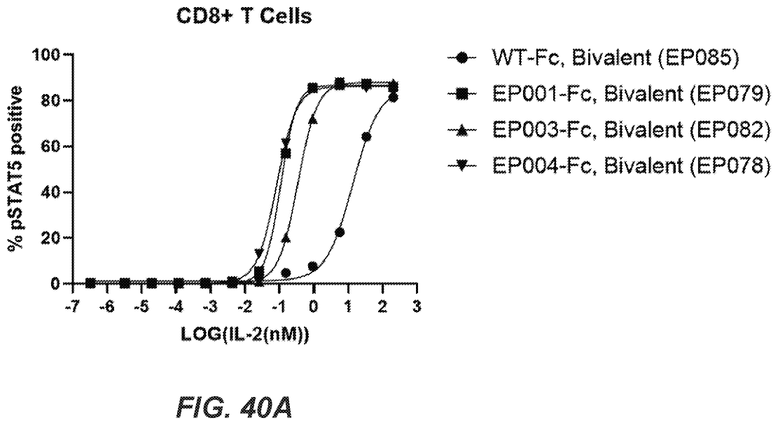

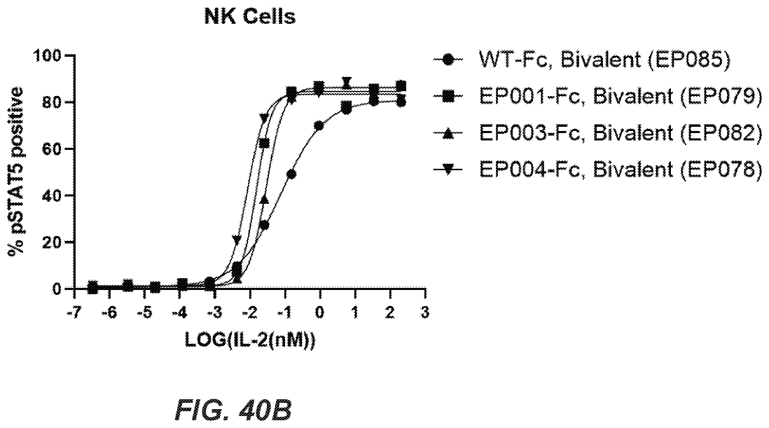

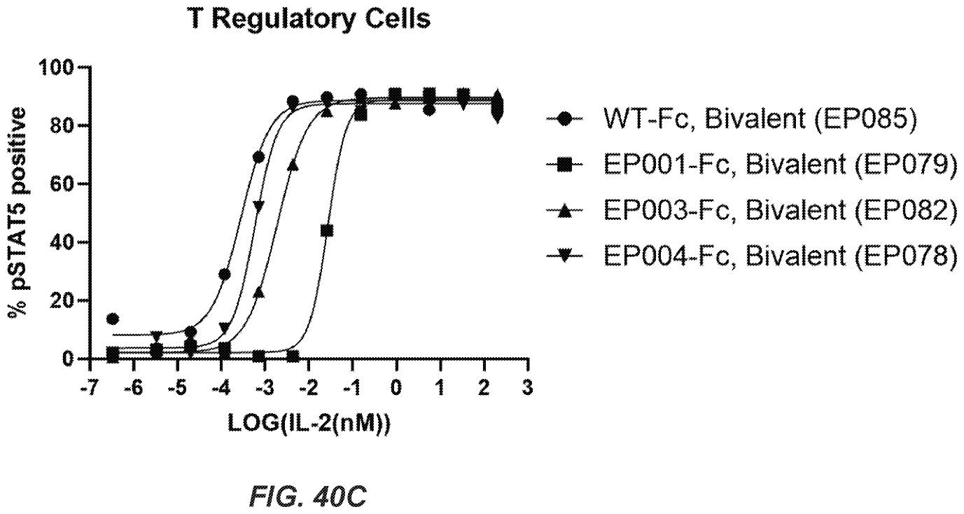

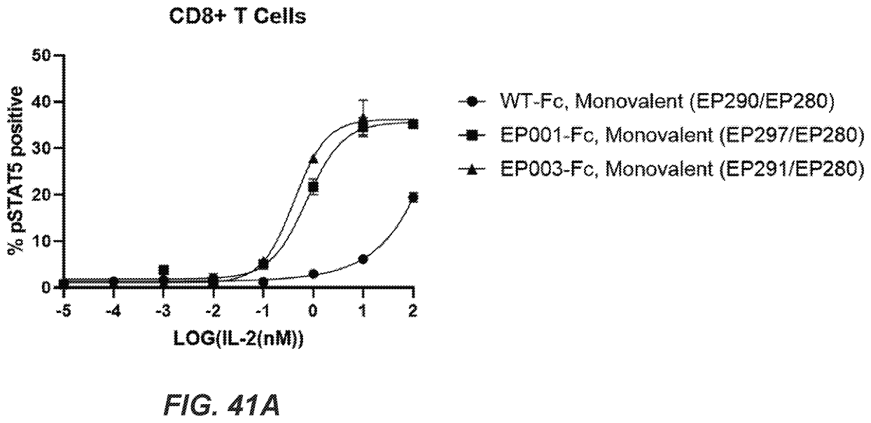

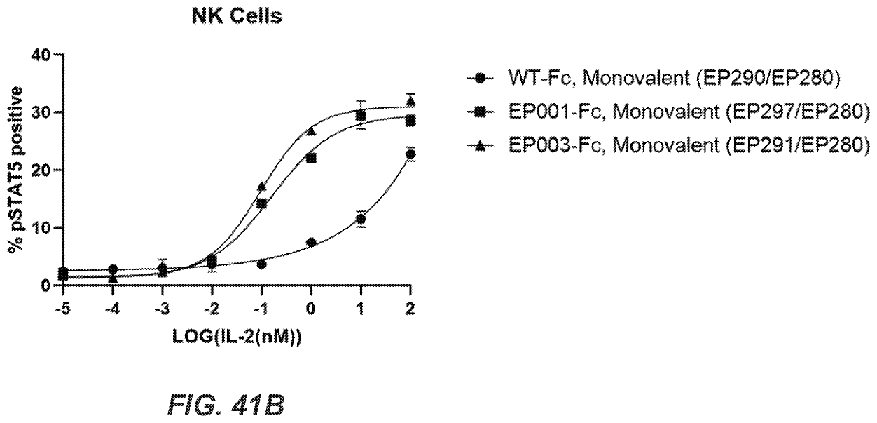

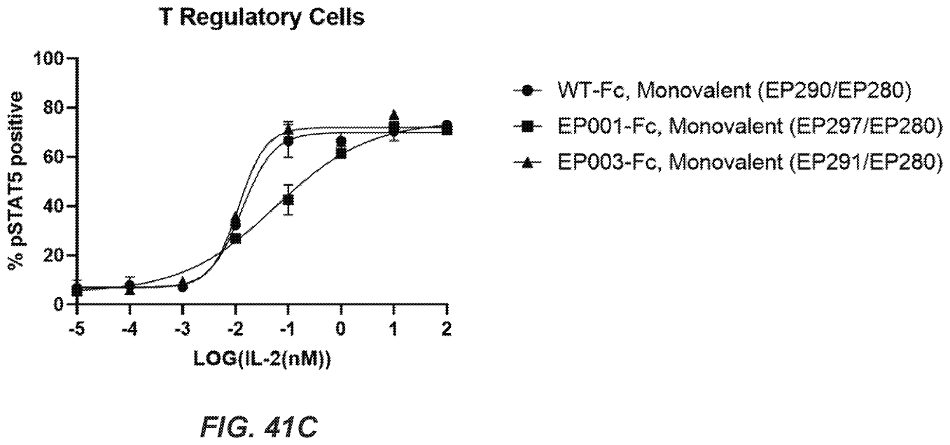

Human PBMCs were isolated from peripheral blood and plated at 250,000 cells/well in a 96-well plate in 75 μl of media. Cells were rested 1 hr at 37° C. Cells were stimulated with human IL2 WT and IL2Rβ agonist Fc-fusion proteins at 4× concentration in 25 μl for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIGS. 40A-40C for bivalent fusion proteins and FIGS. 41A-41C for bivalent fusion proteins; see Table 19 for a summary).

TABLE 19
Summary of P-STAT5 activation of human
PBMCs by IL2Rβ agonist Fc-fusion proteins

| | EC50 (nM) | | |
|---|---|---|---|
| | | | T |
| | CD8+ | NK | Regulatory |
| | T Cells | Cells | Cells |
| WT-Fc, Bivalent (EP085) | 1.4E+01 | 7.7E−02 | 2.8E−04 |
| EP001-Fc, Bivalent (EP079) | 1.1E−01 | 1.5E−02 | 2.7E−02 |
| EP003-Fc, Bivalent (EP082) | 3.4E−01 | 2.9E−02 | 1.9E−03 |
| EP004-Fc, Bivalent (EP078) | 8.4E−02 | 8.5E−03 | 5.9E−04 |
| WT-Fc, Monovalent | >5.0 + 01 | >5.0 + 01 | 1.3E−02 |
| (EP290/EP280) | | | |
| EP001, Monovalent | 7.4E−01 | 1.6E−01 | 5.7E−02 |
| (EP297/EP280) | | | |
| EP003, Monovalent | 4.1E−01 | 9.4E−02 | 1.1E−02 |
| (EP291/EP280) | | | |

### Example 22

### IL2Rβ Agonist Murine In Vivo Pharmacokinetics Analysis

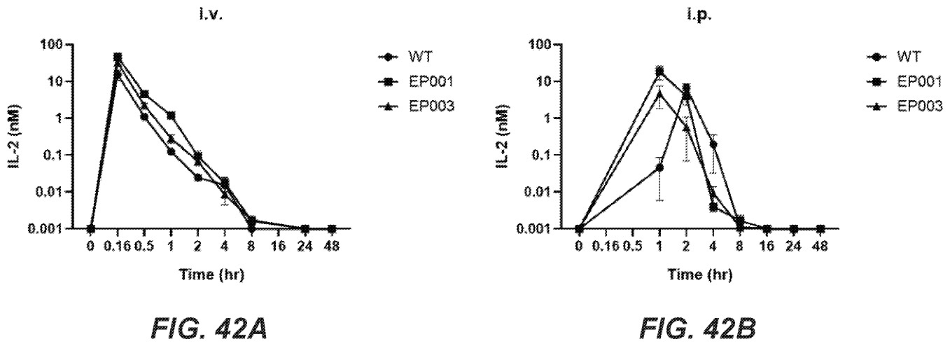

C57BL/6 mice were injected either i.v. or i.p. with 10 ug of IL2-WT, EP001, or EP003 in 200 uL of saline. Blood was collected at 0 min, 10 min, 30 min, 1 hr, 2 hrs, 4 hrs, 8 hrs, 16 hrs, 24 hrs and 48 hrs, and immediately centrifuged to separate out plasma. To determine plasma concentrations of IL2-WT and EP001, plasma was serially diluted and analyzed per instructions using the Duoset IL2 ELISA kit (R&D Systems). IL2-WT, EP001, and EP001 concentrations were determined by comparing absorbance values from plasma to spiked controls made in equally diluted untreated C57BL/6 mouse plasma. IL2 concentration is plotted versus time on a logarithmic scale (FIGS. 42A-42B; Table 20).

TABLE 20

| | Dosing (i.v.) | | | | Dosing (i.p.) | | |
|---|---|---|---|---|---|---|---|
| | WT IL2 | EP001 | EP003 | | WT IL2 | EP001 | EP003 |
| AUC | 81.9 | 266.71 | 170.37 | AUC | 195.59 | 445.38 | 101.3 |
| (ng · h/mL) | | | | (ng · h/mL) | | | |
| Clearance | 113.1 | 34.72 | 54.35 | Cmax | 122.7 | 335.66 | 85.13 |
| (mL/min/kg) | | | | (ng/mL) | | | |
| Volume of | 10.85 | 3.16 | 5.25 | Tmax (h) | 2 | 1 | 1 |
| Distribution | | | | | | | |
| (L/kg) | | | | | | | |
| T1/2 (h) | 1.11 | 1.05 | 1.12 | T1/2 (h) | 0.47 | 0.56 | 0.61 |

### Example 23

### IL2Rβ Agonist Murine In Vivo Tumor Cell Infiltration

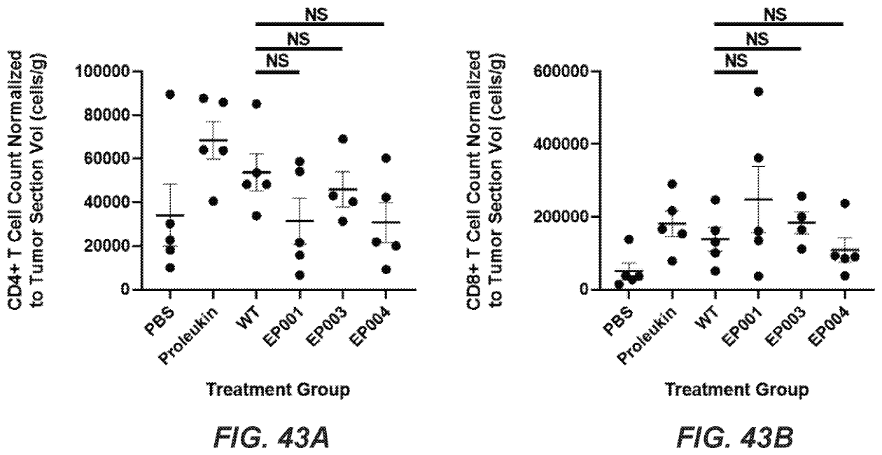

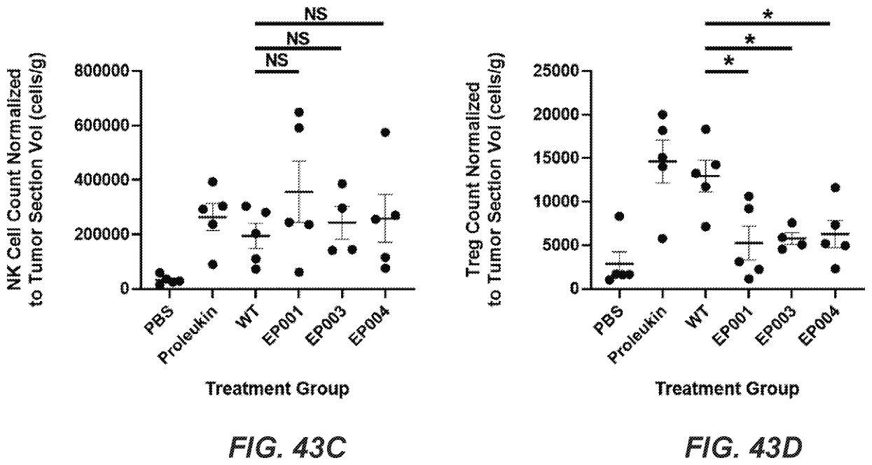

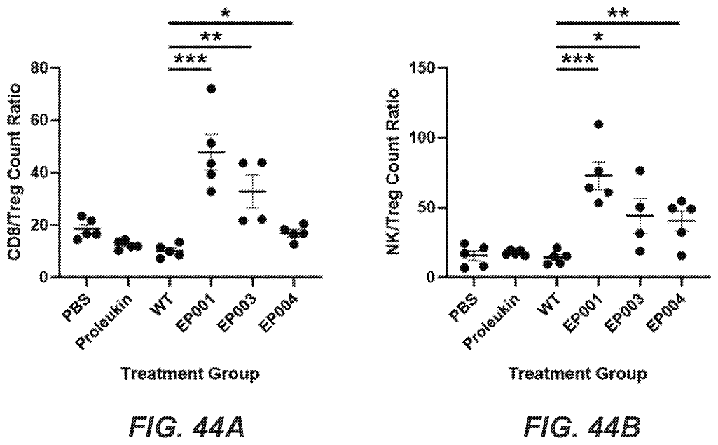

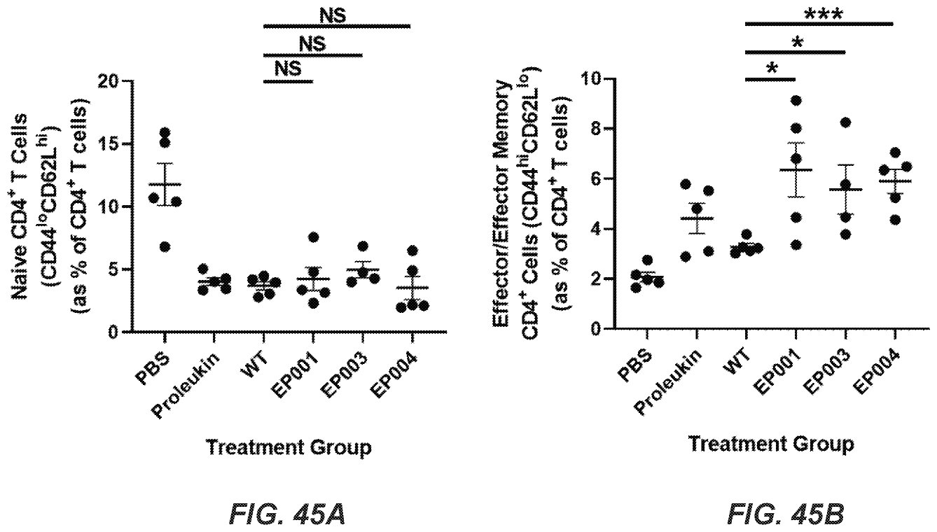

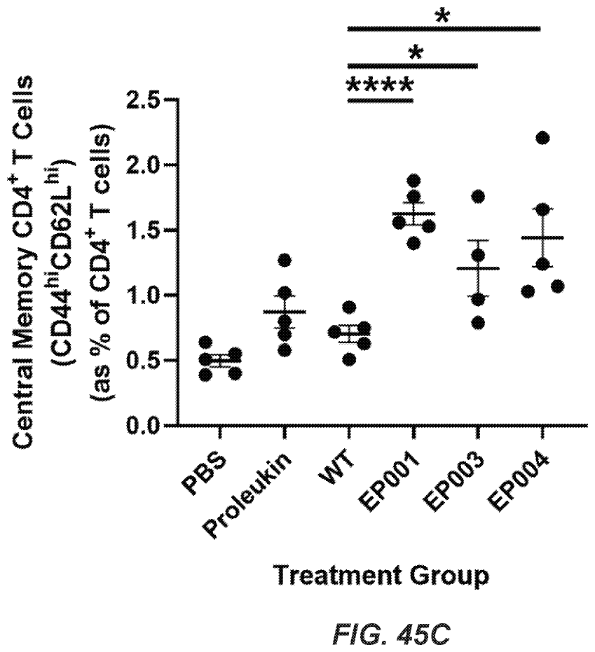

Seven (7)-week old, female C57BL/6 mice were injected with 100,000 MC38 cells in 50% matrigel subcutaneously on their back flank. Tumors were measured with calipers. Upon reaching an average volume of 100 mm³, mice were treated with 32 ug of WT IL2, EP001, EP003, or EP004 BID for 5 days. On day 6, mice were sacrificed and the tumor infiltrating immune cells were analyzed by flow cytometry. Tumor sections used for flow cytometry were weighed to obtain normalized cell counts. CD4+ T cells were defined as CD45+CD3+CD49b−CD4+CD8-. CD8+ T cells were defined as CD45+CD3+CD49b−CD4−CD8+. NK cells were defined as CD45+CD3−CD49b+. T regulatory cells NK cells were defined as CD45+CD3+CD49b−CD4+CD8−FOXP3+. Naïve T cells were defined as CD44^{lo}CD62L^{hi}. Effector T cells were defined as CD44^{hi}CD62L^{lo} and central memory T cells were defined as CD44^{hi}CD62L^{hi}. The normalized counts of tumor infiltrating immune cells (FIGS. 43A-43D), the effector cell to regulatory cell ratios (FIGS. 44A & 44B) and the T cell subtype (FIGS. 45A-45C) are graphed versus IL2 clone treatment group.

1. An engineered interleukin-2 (IL2) polypeptide comprising an engineered IL2 receptor β (IL2Rβ) binding region 2 motif comprising:
(SEQ ID NO: 1)
X₁-X₂-X₃-D-X₄-X₅-X₆-N-X₇-X₈-X₉-X₁₀-X₁₁-X₁₂-X₁₃,
wherein X₁, X₃, X₆, X₈, X₁₂, and X₁₃ each comprise any residue,
wherein X₂, X₄, and X₁₀ are uncharged residues,
wherein X₅, X₇, X₉, and X₁₁ each comprise uncharged, nonpolar residues, and
wherein the engineered IL2 polypeptide binds to IL2Rβ at a K_{D} at least 10-fold greater than a wild-type IL2.

2. (canceled)

3. The engineered IL2 polypeptide of claim 1, wherein:
X₁ is selected from C, T, G, W, I, S, E, and K;
X₂ is selected from Y, P, V, W, L, A, and G;
X₃ is selected from S, T, Q, G, M, E, R, and K;
X₄ is selected from A, V, S, and T;
X₅ is selected from I, L, T, and V;
X₆ is selected from S, T, E, D, and R;
X₇ is selected from I, A, M, and V;
X₈ is selected from S, T, N, Q, I, G, E, K, and R;
X₉ is selected from V, L, and I;
X₁₀ is selected from N, T, I, and L;
X₁₁ is selected from V, A, and I;
X₁₂ is selected from Q, L, G, K, and R; and
X₁₃ is selected from A, D, and E.

4.-35. (canceled)

36. The engineered IL2 polypeptide of claim 1, wherein the engineered IL2Rβ binding region 2 is selected from: GVTDSISNAIVLARE (SEQ ID NO: 2); KWGDAVSNARVLAGE (SEQ ID NO: 3); KWGDAVSNARVLAGA (SEQ ID NO: 4); TLMDTTDNIGVLVRE (SEQ ID NO: 5); EPSDVISNINVLVQE (SEQ ID NO: 6); SPQDSIENISVLVRE (SEQ ID NO: 7); WASDSIENITLLIQE (SEQ ID NO: 8); CPTDTIENITVLIQE (SEQ ID NO: 9); RYKDSLENMQIIIQE (SEQ ID NO: 10); TARDAVDNMRVIIQE (SEQ ID NO: 11); TPRDVVENMNVLVLE (SEQ ID NO: 12); TPSDVIENMEVLILD (SEQ ID NO: 13); TPSDAIENINVLIRE (SEQ ID NO: 14); TPSDVIENITVLVQE (SEQ ID NO: 15); GVGDTIDNINVLVKE (SEQ ID NO: 16); IGRDSIDNIKVIVQE (SEQ ID NO: 17); WATDTIRNVEVLVQE (SEQ ID NO: 18); TAEDVVTNITVLVQE (SEQ ID NO: 19); TAEDVISNIRVNVQE (SEQ ID NO: 20); and TPSDVIDNVSITVQE (SEQ ID NO: 21); TARDAISNIRVIVQE (SEQ ID NO: 210); RARDAIDNIRVIVQE (SEQ ID NO: 211); TPRDAIDNINVIIQE (SEQ ID NO: 212); TPRDAIDNIRVIVQE (SEQ ID NO: 213); TPRDAIDNIRVIILE (SEQ ID NO: 214); TARDAISNINVIIQE (SEQ ID NO: 215); and TARDAIDNINVIVQE (SEQ ID NO: 216); and TARDAIDNIRVIVLE (SEQ ID NO: 217).

37.-53. (canceled)

54. The engineered IL2 polypeptide of claim 1, wherein the engineered IL2 polypeptide has a decrease in affinity for IL2Rα as compared to wild-type IL2.

55. (canceled)

56. An engineered interleukin-2 (IL2) polypeptide comprising an engineered IL2 receptor α (IL2Rα) binding region 1 comprising a substitution selected from: a substitution at position K35, a substitution at R38, a substitution at F42, a substitution at Y45, or any combination thereof, wherein the engineered IL2 polypeptide binds to IL2Rα with at least 2-fold reduced binding kinetics as compared to wild-type IL2.

57.-81. (canceled)

82. The engineered IL2 polypeptide of claim 56, comprising a substitution at least one of positions K35, R38, F42, and Y45, wherein:
i) the substitution at position K35 is selected from: K35G, K35L, K35S, K35V, K35D, K35E, and K35C;
ii) the substitution at position R38 is selected from: R38V, R38D, R38E, R38S, R38I, R38A, R38Y, R38G, R38C, or R3 8N;
iii) the substitution at position F42 is selected from: F42A, F42R, F42G, F42I, F42L, F42P and F42H; and
iv) the substitution at position Y45 is Y45S, Y45P, Y45A, Y45V, Y45C, Y45T, and Y45F.

83.-84. (canceled)

85. The engineered IL2 polypeptide claim 56, wherein the engineered IL2Rα binding region 1 is selected from:
(SEQ ID NO: 183)
PVLTRMLTIKFY;
(SEQ ID NO: 184)
PKLTRMLTLKFP;
(SEQ ID NO: 185)
PDLTSMLAFKFY;
(SEQ ID NO: 186)
PGLTEMLTFKFY;
(SEQ ID NO: 187)
PSLTRMLTGKFY;
(SEQ ID NO: 188)
PELTIMLTPKFY;
(SEQ ID NO: 189)
PCLTAMLTLKFA;
(SEQ ID NO: 190)
PCLTAMLTLKFA;
(SEQ ID NO: 191)
PKLTRMLTHKFV;
(SEQ ID NO: 192)
PCLTDMLTFKFY;
(SEQ ID NO: 193)
PLLTDMLTRKFY;
(SEQ ID NO: 194)
PLLTDMLTFKFY;
(SEQ ID NO: 195)
PKLTDMLTFKFS;
(SEQ ID NO: 196)
PKLTYMLTRKFY;
(SEQ ID NO: 197)
PKLTRMLTFKFC;
(SEQ ID NO: 198)
PKLTSMLTFKFS;
(SEQ ID NO: 199)
PKLTSMLTFKFS;
(SEQ ID NO: 200)
PKLTYMLTFKFS;
(SEQ ID NO: 201)
PKLTYMLTFKFS;
(SEQ ID NO: 202)
PKLTGMLTFKFS;
(SEQ ID NO: 203)
PKLTVMLTFKFT;
(SEQ ID NO: 204)
PKLTYMLTFKFS;
(SEQ ID NO: 205)
PKLTVMLTFKFP;
(SEQ ID NO: 206)
PKLTVMLTFKFF;
(SEQ ID NO: 207)
PKLTCMLTFKFA;
(SEQ ID NO: 208)
PKLTNMLTFKFA;
and
(SEQ ID NO: 209)
PKLTNMLTFKFS.

86. (canceled)

87. The engineered IL2 polypeptide of claim 1, further comprising an engineered IL2Rα binding region 1, wherein the engineered IL2Rα binding region 1 is selected from: PVLTRMLTIKFY (SEQ ID NO: 183); PKLTRMLTLKFP (SEQ ID NO:184); PDLTSMLAFKFY (SEQ ID NO:185); PGLTEMLTFKFY(SEQ ID NO:186); PSLTRMLTGKFY (SEQ ID NO:187); PELTIMLTPKFY(SEQ ID NO:188); PCLTAMLTLKFA (SEQ ID NO:189); PCLTAMLTLKFA (SEQ ID NO:190); PKLTRMLTHKFV (SEQ ID NO:191); PCLTDMLTFKFY(SEQ ID NO:192); PLLTDMLTRKFY (SEQ ID NO:193); PLLTDMLTFKFY(SEQ ID NO:194); PKLTDMLTFKFS (SEQ ID NO:195); PKLTYMLTRKFY(SEQ ID NO:196); PKLTRMLTFKFC (SEQ ID NO:197); PKLTSMLTFKFS(SEQ ID NO:198); PKLTSMLTFKFS (SEQ ID NO:199); PKLTYMLTFKFS(SEQ ID NO:200); PKLTYMLTFKFS (SEQ ID NO:201); PKLTGMLTFKFS(SEQ ID NO:202); PKLTVMLTFKFT (SEQ ID NO:203); PKLTVMLTFKFS(SEQ ID NO:204); PKLTVMLTFKFP (SEQ ID NO:205); PKLTVMLTFKFF (SEQ ID NO:206); PKLTCMLTFKFA (SEQ ID NO:207); PKLTNMLTFKFA (SEQ ID NO:208); and PKLTNMLTFKFS (SEQ ID NO:209); and wherein the engineered IL2Rβ binding region 2 is selected from: GVTDSISNAIVLARE (SEQ ID NO: 2); KWGDAVSNARVLAGE (SEQ ID NO: 3); KWGDAVSNARVLAGA (SEQ ID NO: 4); TLMDTTDNIGVLVRE (SEQ ID NO: 5); EPSDVISNINVLVQE (SEQ ID NO: 6); SPQDSIENISVLVRE (SEQ ID NO: 7); WASDSIENITLLIQE (SEQ ID NO: 8); CPTDTIENITVLIQE (SEQ ID NO: 9); RYKDSLENMQIIIQE (SEQ ID NO: 10); TARDAVDNMRVIIQE (SEQ ID NO: 11); TPRDVVENMNVLVLE (SEQ ID NO: 12); TPSDVIENMEVLILD (SEQ ID NO: 13); TPSDAIENINVLIRE (SEQ ID NO: 14); TPSDVIENITVLVQE (SEQ ID NO: 15); GVGDTIDNINVLVKE (SEQ ID NO: 16); IGRDSIDNIKVIVQE (SEQ ID NO: 17); WATDTIRNVEVLVQE (SEQ ID NO: 18); TAEDVVTNITVLVQE (SEQ ID NO: 19); TAEDVISNIRVNVQE (SEQ ID NO: 20); and TPSDVIDNVSITVQE (SEQ ID NO: 21); TARDAISNIRVIVQE (SEQ ID NO: 210); RARDAIDNIRVIVQE (SEQ ID NO: 211); TPRDAIDNINVIIQE (SEQ ID NO: 212); TPRDAIDNIRVIVQE (SEQ ID NO: 213); TPRDAIDNIRVIILE (SEQ ID NO: 214); TARDAISNINVIIQE (SEQ ID NO: 215); and TARDAIDNINVIVQE (SEQ ID NO: 216); and TARDAIDNIRVIVLE (SEQ ID NO: 217).

88. A fusion polypeptide comprising an engineered IL2 polypeptide of claim 1 fused to a half-life extending molecule.

89. (canceled)

90. The fusion polypeptide of claim 88, wherein the half-life extending molecule comprises a half-life extending polypeptide, and the half-life extending polypeptide comprises an Fc domain, human serum albumin (HSA), an HSA binding molecule, or transferrin.

91. (canceled)

92. The fusion polypeptide of claim 88, wherein the half-life extending molecule comprises poly-ethylene glycol (PEG) or polypropylene glycol (PPG).

93. A fusion polypeptide comprising a first polypeptide and a second polypeptide, wherein the first polypeptide comprises an engineered IL2 polypeptide of claim 1.

94. The fusion polypeptide of claim 93, wherein the second polypeptide comprises an antigen binding moiety.

95. (canceled)

96. The fusion polypeptide of claim 94, wherein the antigen binding moiety comprises a Fab molecule, an scFv, a bi-specific T-cell engager, a diabody, a single domain antibody, or a nanobody.

97. The fusion polypeptide of claim 93, wherein the second polypeptide comprises a cytokine.

98. The fusion polypeptide of claim 97, wherein the second polypeptide comprises interleukin 2, interleukin-15, interleukin-7, interleukin-10, or C-C motif chemokine ligand 19 (CCL19).

99. (canceled)

100. An isolated polynucleotide encoding at least one polypeptide of claim 1.

101. An expression vector comprising the polynucleotide of claim 100.

102. A modified cell comprising the isolated polynucleotide of claim 100.

103. The modified cell of claim 102 further comprising an engineered T cell receptor or chimeric antigen receptor.

104. A pharmaceutical composition comprising the engineered IL2 polypeptide of claim 1 and a pharmaceutically acceptable carrier.

105. A method of modulating an immune response in a subject in need thereof, comprising administering an effective amount of an engineered IL2 polypeptide of claim 1 to the subject.

106. The method of claim 105, wherein the modulating the immune response comprises at least one of: enhancing effector T cell activity, enhancing NK cell activity, and suppressing regulatory T cell activity.

107. A method of treating a disease in a subject in need thereof, comprising administering an effective amount of an engineered IL2 polypeptide of claim 1 to the subject.

108. The method of claim 107, wherein the disease comprises cancer or immunosuppression.

109. The method of claim 108, wherein the cancer comprises breast cancer, pancreatic cancer, lung cancer, glioblastoma, renal cell carcinoma, or melanoma.

110.-120. (canceled)
